SEQUENCE LISTING (120 sequences)

SEQ ID NO: 1 - protein, 1023 aa, Homo sapiens
MGKGVGRDKYEPAAVSEQGDKKGKKGKKDRDMDELKKEVSMDDHKLSLDELHRKYGTDLSRGLTSARAAEILARDGPNALTPPPTTPEWIKFCRQLFGGFSMLLWIGAILCFLAYSIQAATEEEPQNDNLYLGVVLSAVVIITGCFSYYQEAKSSKIMESFKNMVPQQALVIRNGEKMSINAEEVVVGDLVEVKGGDRIPADLRIISANGCKVDNSSLTGESEPQTRSPDFTNENPLETRNIAFFSTNCVEGTARGIVVYTGDRTVMGRIATLASGLEGGQTPIAAEIEHFIHIITGVAVFLGVSFFILSLILEYTWLEAVIFLIGIIVANVPEGLLATVTVCLTLTAKRMARKNCLVKNLEAVETLGSTSTICSDKTGTLTQNRMTVAHMWFDNQIHEADTTENQSGVSFDKTSATWLALSRIAGLCNRAVFQANQENLPILKRAVAGDASESALLKCIELCCGSVKEMRERYAKIVEIPFNSTNKYQLSIHKNPNTSEPQHLLVMKGAPERILDRCSSILLHGKEQPLDEELKDAFQNAYLELGGLGERVLGFCHLFLPDEQFPEGFQFDTDDVNFPIDNLCFVGLISMIDPPRAAVPDAVGKCRSAGIKVIMVTGDHPITAKAIAKGVGIISEGNETVEDIAARLNIPVSQVNPRDAKACVVHGSDLKDMTSEQLDDILKYHTEIVFARTSPQQKLIIVEGCQRQGAIVAVTGDGVNDSPALKKADIGVAMGIAGSDVSKQAADMILLDDNFASIVTGVEEGRLIFDNLKKSIAYTLTSNIPEITPFLIFIIANIPLPLGTVTILCIDLGTDMVPAISLAYEQAESDIMKRQPRNPKTDKLVNERLISMAYGQIGMIQALGGFFTYFVILAENGFLPIHLLGLRVDWDDRWINDVEDSYGQQWTYEQRKIVEFTCHTAFFVSIVVVQWADLVICKTRRNSVFQQGMKNKILIFGLFEETALAAFLSYCPGMGVALRMYPLKPTWWFCAFPYSLLIFVYDEVRKLIIRRRPGGWVEKETYY

SEQ ID NO: 2 - protein, 1023 aa, Homo sapiens
MAFKVGRDKYEPAAVSEQGDKKGKKGKKDRDMDELKKEVSMDDHKLSLDELHRKYGTDLSRGLTSARAAEILARDGPNALTPPPTTPEWIKFCRQLFGGFSMLLWIGAILCFLAYSIQAATEEEPQNDNLYLGVVLSAVVIITGCFSYYQEAKSSKIMESFKNMVPQQALVIRNGEKMSINAEEVVVGDLVEVKGGDRIPADLRIISANGCKVDNSSLTGESEPQTRSPDFTNENPLETRNIAFFSTNCVEGTARGIVVYTGDRTVMGRIATLASGLEGGQTPIAAEIEHFIHIITGVAVFLGVSFFILSLILEYTWLEAVIFLIGIIVANVPEGLLATVTVCLTLTAKRMARKNCLVKNLEAVETLGSTSTICSDKTGTLTQNRMTVAHMWFDNQIHEADTTENQSGVSFDKTSATWLALSRIAGLCNRAVFQANQENLPILKRAVAGDASESALLKCIELCCGSVKEMRERYAKIVEIPFNSTNKYQLSIHKNPNTSEPQHLLVMKGAPERILDRCSSILLHGKEQPLDEELKDAFQNAYLELGGLGERVLGFCHLFLPDEQFPEGFQFDTDDVNFPIDNLCFVGLISMIDPPRAAVPDAVGKCRSAGIKVIMVTGDHPITAKAIAKGVGIISEGNETVEDIAARLNIPVSQVNPRDAKACVVHGSDLKDMTSEQLDDILKYHTEIVFARTSPQQKLIIVEGCQRQGAIVAVTGDGVNDSPALKKADIGVAMGIAGSDVSKQAADMILLDDNFASIVTGVEEGRLIFDNLKKSIAYTLTSNIPEITPFLIFIIANIPLPLGTVTILCIDLGTDMVPAISLAYEQAESDIMKRQPRNPKTDKLVNERLISMAYGQIGMIQALGGFFTYFVILAENGFLPIHLLGLRVDWDDRWINDVEDSYGQQWTYEQRKIVEFTCHTAFFVSIVVVQWADLVICKTRRNSVFQQGMKNKILIFGLFEETALAAFLSYCPGMGVALRMYPLKPTWWFCAFPYSLLIFVYDEVRKLIIRRRPGGWVEKETYY

SEQ ID NO: 3 - protein, 992 aa, Homo sapiens
MDELKKEVSMDDHKLSLDELHRKYGTDLSRGLTSARAAEILARDGPNALTPPPTTPEWIKFCRQLFGGFSMLLWIGAILCFLAYSIQAATEEEPQNDNLYLGVVLSAVVIITGCFSYYQEAKSSKIMESFKNMVPQQALVIRNGEKMSINAEEVVVGDLVEVKGGDRIPADLRIISANGCKVDNSSLTGESEPQTRSPDFTNENPLETRNIAFFSTNCVEGTARGIVVYTGDRTVMGRIATLASGLEGGQTPIAAEIEHFIHIITGVAVFLGVSFFILSLILEYTWLEAVIFLIGIIVANVPEGLLATVTVCLTLTAKRMARKNCLVKNLEAVETLGSTSTICSDKTGTLTQNRMTVAHMWFDNQIHEADTTENQSGVSFDKTSATWLALSRIAGLCNRAVFQANQENLPILKRAVAGDASESALLKCIELCCGSVKEMRERYAKIVEIPFNSTNKYQLSIHKNPNTSEPQHLLVMKGAPERILDRCSSILLHGKEQPLDEELKDAFQNAYLELGGLGERVLGFCHLFLPDEQFPEGFQFDTDDVNFPIDNLCFVGLISMIDPPRAAVPDAVGKCRSAGIKVIMVTGDHPITAKAIAKGVGIISEGNETVEDIAARLNIPVSQVNPRDAKACVVHGSDLKDMTSEQLDDILKYHTEIVFARTSPQQKLIIVEGCQRQGAIVAVTGDGVNDSPALKKADIGVAMGIAGSDVSKQAADMILLDDNFASIVTGVEEGRLIFDNLKKSIAYTLTSNIPEITPFLIFIIANIPLPLGTVTILCIDLGTDMVPAISLAYEQAESDIMKRQPRNPKTDKLVNERLISMAYGQIGMIQALGGFFTYFVILAENGFLPIHLLGLRVDWDDRWINDVEDSYGQQWTYEQRKIVEFTCHTAFFVSIVVVQWADLVICKTRRNSVFQQGMKNKILIFGLFEETALAAFLSYCPGMGVALRMYPLKPTWWFCAFPYSLLIFVYDEVRKLIIRRRPGGWVEKETYY

SEQ ID NO: 4 - DNA, 3670 bp, Homo sapiens
  source 1..3670: mol_type = genomic DNA
ggagctgcggcggggtctggggcgcagagcagcggcgggaggaggcggacacgtggcaacagcggtagcagcccgggcggcggcagcaacagcggcggcggcatcggcccgagccgccggccgccctcccaccctcccgccccgcggcagccctagctccctccacttggctcccctggtcccgctcgctcggccgggagctgctctgtgcttttctctctgattctccagcgacaggacccggcgccgggcactgagcaccgccaccatggggaagggggttggacgtgataagtatgagcctgcagctgtttcagaacaaggtgataaaaagggcaaaaagggcaaaaaagacagggacatggatgaactgaagaaagaagtttctatggatgatcataaacttagccttgatgaacttcatcgtaaatatggaacagacttgagccggggattaacatctgctcgtgcagctgagatcctggcgcgagatggtcccaacgccctcactccccctcccactactcctgaatggatcaagttttgtcggcagctctttggggggttctcaatgttactgtggattggagcgattctttgtttcttggcttatagcatccaagctgctacagaagaggaacctcaaaacgataatctgtacctgggtgtggtgctatcagccgttgtaatcataactggttgcttctcctactatcaagaagctaaaagttcaaagatcatggaatccttcaaaaacatggtccctcagcaagcccttgtgattcgaaatggtgagaaaatgagcataaatgcggaggaagttgtggttggggatctggtggaagtaaaaggaggagaccgaattcctgctgacctcagaatcatatctgcaaatggctgcaaggtggataactcctcgctcactggtgaatcagaaccccagactaggtctccagatttcacaaatgaaaaccccctggagacgaggaacattgccttcttttcaaccaattgtgttgaaggcaccgcacgtggtattgttgtctacactggggatcgcactgtgatgggaagaattgccacacttgcttctgggctggaaggaggccagacccccattgctgcagaaattgaacattttatccacatcatcacgggtgtggctgtgttcctgggtgtgtctttcttcatcctttctctcatccttgagtacacctggcttgaggctgtcatcttcctcatcggtatcatcgtagccaatgtgccggaaggtttgctggccactgtcacggtctgtctgacacttactgccaaacgcatggcaaggaaaaactgcttagtgaagaacttagaagctgtggagaccttggggtccacgtccaccatctgctctgataaaactggaactctgactcagaaccggatgacagtggcccacatgtggtttgacaatcaaatccatgaagctgatacgacagagaatcagagtggtgtctcttttgacaagacttcagctacctggcttgctctgtccagaattgcaggtctttgtaacagggcagtgtttcaggctaaccaggaaaacctacctattcttaagcgggcagttgcaggagatgcctctgagtcagcactcttaaagtgcatagagctgtgctgtggttccgtgaaggagatgagagaaagatacgccaaaatcgtcgagatacccttcaactccaccaacaagtaccagttgtctattcataagaaccccaacacatcggagccccaacacctgttggtgatgaagggcgccccagaaaggatcctagaccgttgcagctctatcctcctccacggcaaggagcagcccctggatgaggagctgaaagacgcctttcagaacgcctatttggagctggggggcctcggagaacgagtcctaggtttctgccacctctttctgccagatgaacagtttcctgaagggttccagtttgacactgacgatgtgaatttccctatcgataatctgtgctttgttgggctcatctccatgattgaccctccacgggcggccgttcctgatgccgtgggcaaatgtcgaagtgctggaattaaggtcatcatggtcacaggagaccatccaatcacagctaaagctattgccaaaggtgtgggcatcatctcagaaggcaatgagaccgtggaagacattgctgcccgcctcaacatcccagtcagccaggtgaaccccagggatgccaaggcctgcgtagtacacggcagtgatctaaaggacatgacctccgagcagctggatgacattttgaagtaccacactgagatagtgtttgccaggacctcccctcagcagaagctcatcattgtggaaggctgccaaagacagggtgctatcgtggctgtgactggtgacggtgtgaatgactctccagctttgaagaaagcagacattggggttgctatggggattgctggctcagatgtgtccaagcaagctgctgacatgattcttctggatgacaactttgcctcaattgtgactggagtagaggaaggtcgtctgatctttgataacttgaagaaatccattgcttataccttaaccagtaacattcccgagatcaccccgttcctgatatttattattgcaaacattccactaccactggggactgtcaccatcctctgcattgacttgggcactgacatggttcctgccatctccctggcttatgagcaggctgagagtgacatcatgaagagacagcccagaaatcccaaaacagacaaacttgtgaatgagcggctgatcagcatggcctatgggcagattggaatgatccaggccctgggaggcttctttacttactttgtgattctggctgagaacggcttcctcccaattcacctgttgggcctccgagtggactgggatgaccgctggatcaacgatgtggaagacagctacgggcagcagtggacctatgagcagaggaaaatcgtggagttcacctgccacacagccttcttcgtcagtatcgtggtggtgcagtgggccgacttggtcatctgtaagaccaggaggaattcggtcttccagcaggggatgaagaacaagatcttgatatttggcctctttgaagagacagccctggctgctttcctttcctactgccctggaatgggtgttgctcttaggatgtatcccctcaaacctacctggtggttctgtgccttcccctactctcttctcatcttcgtatatgacgaagtcagaaaactcatcatcaggcgacgccctggcggctgggtggagaaggaaacctactattagccccccgtcctgcacgccgtggagcatcaggccacacactctgcatccgacacccaccccctctttgtgtacttcagtcttggagtttggaactctaccctggtaggaaagcaccgcagcatgtggggaagcaagacgtcctggaatgaagcatgtagctctatggggggaggggggagggctgcctgaaaaccatccatctgtggaaatgacagcggggaaggtttttatgtgcctttttgtttttgtaaaaaaggaacacccggaaagactgaaagaatacattttatatctggatttttacaaataaagatggctattataatggaa

SEQ ID NO: 5 - DNA, 3765 bp, Homo sapiens
  source 1..3765: mol_type = genomic DNA
agcagcgggggcggccccgggactgagccggcatccctgagcctggctcccctccctgcgaccgccgtcacctccttctccttccttttccctccgccctccgtgccctgaggaaaggcgcgctcctccccttcccctggggcgctccgccggggcctcctcccgggcctccgttcccgccgcggccccggttccggcgggggcagcctccgggttcggggctccttctcctggggacgctggggcttagcttgctccgcgcagaggcggccgccctcccccaaagaaaaaactggctgcttctaagtgcgaagccggctgggcgggctggtgccagaaagggtgtgtcttcactgccctaagatggcctttaaggttggacgtgataagtatgagcctgcagctgtttcagaacaaggtgataaaaagggcaaaaagggcaaaaaagacagggacatggatgaactgaagaaagaagtttctatggatgatcataaacttagccttgatgaacttcatcgtaaatatggaacagacttgagccggggattaacatctgctcgtgcagctgagatcctggcgcgagatggtcccaacgccctcactccccctcccactactcctgaatggatcaagttttgtcggcagctctttggggggttctcaatgttactgtggattggagcgattctttgtttcttggcttatagcatccaagctgctacagaagaggaacctcaaaacgataatctgtacctgggtgtggtgctatcagccgttgtaatcataactggttgcttctcctactatcaagaagctaaaagttcaaagatcatggaatccttcaaaaacatggtccctcagcaagcccttgtgattcgaaatggtgagaaaatgagcataaatgcggaggaagttgtggttggggatctggtggaagtaaaaggaggagaccgaattcctgctgacctcagaatcatatctgcaaatggctgcaaggtggataactcctcgctcactggtgaatcagaaccccagactaggtctccagatttcacaaatgaaaaccccctggagacgaggaacattgccttcttttcaaccaattgtgttgaaggcaccgcacgtggtattgttgtctacactggggatcgcactgtgatgggaagaattgccacacttgcttctgggctggaaggaggccagacccccattgctgcagaaattgaacattttatccacatcatcacgggtgtggctgtgttcctgggtgtgtctttcttcatcctttctctcatccttgagtacacctggcttgaggctgtcatcttcctcatcggtatcatcgtagccaatgtgccggaaggtttgctggccactgtcacggtctgtctgacacttactgccaaacgcatggcaaggaaaaactgcttagtgaagaacttagaagctgtggagaccttggggtccacgtccaccatctgctctgataaaactggaactctgactcagaaccggatgacagtggcccacatgtggtttgacaatcaaatccatgaagctgatacgacagagaatcagagtggtgtctcttttgacaagacttcagctacctggcttgctctgtccagaattgcaggtctttgtaacagggcagtgtttcaggctaaccaggaaaacctacctattcttaagcgggcagttgcaggagatgcctctgagtcagcactcttaaagtgcatagagctgtgctgtggttccgtgaaggagatgagagaaagatacgccaaaatcgtcgagatacccttcaactccaccaacaagtaccagttgtctattcataagaaccccaacacatcggagccccaacacctgttggtgatgaagggcgccccagaaaggatcctagaccgttgcagctctatcctcctccacggcaaggagcagcccctggatgaggagctgaaagacgcctttcagaacgcctatttggagctggggggcctcggagaacgagtcctaggtttctgccacctctttctgccagatgaacagtttcctgaagggttccagtttgacactgacgatgtgaatttccctatcgataatctgtgctttgttgggctcatctccatgattgaccctccacgggcggccgttcctgatgccgtgggcaaatgtcgaagtgctggaattaaggtcatcatggtcacaggagaccatccaatcacagctaaagctattgccaaaggtgtgggcatcatctcagaaggcaatgagaccgtggaagacattgctgcccgcctcaacatcccagtcagccaggtgaaccccagggatgccaaggcctgcgtagtacacggcagtgatctaaaggacatgacctccgagcagctggatgacattttgaagtaccacactgagatagtgtttgccaggacctcccctcagcagaagctcatcattgtggaaggctgccaaagacagggtgctatcgtggctgtgactggtgacggtgtgaatgactctccagctttgaagaaagcagacattggggttgctatggggattgctggctcagatgtgtccaagcaagctgctgacatgattcttctggatgacaactttgcctcaattgtgactggagtagaggaaggtcgtctgatctttgataacttgaagaaatccattgcttataccttaaccagtaacattcccgagatcaccccgttcctgatatttattattgcaaacattccactaccactggggactgtcaccatcctctgcattgacttgggcactgacatggttcctgccatctccctggcttatgagcaggctgagagtgacatcatgaagagacagcccagaaatcccaaaacagacaaacttgtgaatgagcggctgatcagcatggcctatgggcagattggaatgatccaggccctgggaggcttctttacttactttgtgattctggctgagaacggcttcctcccaattcacctgttgggcctccgagtggactgggatgaccgctggatcaacgatgtggaagacagctacgggcagcagtggacctatgagcagaggaaaatcgtggagttcacctgccacacagccttcttcgtcagtatcgtggtggtgcagtgggccgacttggtcatctgtaagaccaggaggaattcggtcttccagcaggggatgaagaacaagatcttgatatttggcctctttgaagagacagccctggctgctttcctttcctactgccctggaatgggtgttgctcttaggatgtatcccctcaaacctacctggtggttctgtgccttcccctactctcttctcatcttcgtatatgacgaagtcagaaaactcatcatcaggcgacgccctggcggctgggtggagaaggaaacctactattagccccccgtcctgcacgccgtggagcatcaggccacacactctgcatccgacacccaccccctctttgtgtacttcagtcttggagtttggaactctaccctggtaggaaagcaccgcagcatgtggggaagcaagacgtcctggaatgaagcatgtagctctatggggggaggggggagggctgcctgaaaaccatccatctgtggaaatgacagcggggaaggtttttatgtgcctttttgtttttgtaaaaaaggaacacccggaaagactgaaagaatacattttatatctggatttttacaaataaagatggctattataatggaa

SEQ ID NO: 6 - DNA, 3574 bp, Homo sapiens
  source 1..3574: mol_type = genomic DNA
gatatgtaataatgtctttgcaaagcaaagaatataaacagtataaaagtactagcatttagatgtattgtatcatttaatccttaaaaacatgaaatgaggttggcactattctttatctccacgctgtggaagaggaaattgaaatgtagaagttagtaacttgcctaaggatacactgctggttggacgtgataagtatgagcctgcagctgtttcagaacaaggtgataaaaagggcaaaaagggcaaaaaagacagggacatggatgaactgaagaaagaagtttctatggatgatcataaacttagccttgatgaacttcatcgtaaatatggaacagacttgagccggggattaacatctgctcgtgcagctgagatcctggcgcgagatggtcccaacgccctcactccccctcccactactcctgaatggatcaagttttgtcggcagctctttggggggttctcaatgttactgtggattggagcgattctttgtttcttggcttatagcatccaagctgctacagaagaggaacctcaaaacgataatctgtacctgggtgtggtgctatcagccgttgtaatcataactggttgcttctcctactatcaagaagctaaaagttcaaagatcatggaatccttcaaaaacatggtccctcagcaagcccttgtgattcgaaatggtgagaaaatgagcataaatgcggaggaagttgtggttggggatctggtggaagtaaaaggaggagaccgaattcctgctgacctcagaatcatatctgcaaatggctgcaaggtggataactcctcgctcactggtgaatcagaaccccagactaggtctccagatttcacaaatgaaaaccccctggagacgaggaacattgccttcttttcaaccaattgtgttgaaggcaccgcacgtggtattgttgtctacactggggatcgcactgtgatgggaagaattgccacacttgcttctgggctggaaggaggccagacccccattgctgcagaaattgaacattttatccacatcatcacgggtgtggctgtgttcctgggtgtgtctttcttcatcctttctctcatccttgagtacacctggcttgaggctgtcatcttcctcatcggtatcatcgtagccaatgtgccggaaggtttgctggccactgtcacggtctgtctgacacttactgccaaacgcatggcaaggaaaaactgcttagtgaagaacttagaagctgtggagaccttggggtccacgtccaccatctgctctgataaaactggaactctgactcagaaccggatgacagtggcccacatgtggtttgacaatcaaatccatgaagctgatacgacagagaatcagagtggtgtctcttttgacaagacttcagctacctggcttgctctgtccagaattgcaggtctttgtaacagggcagtgtttcaggctaaccaggaaaacctacctattcttaagcgggcagttgcaggagatgcctctgagtcagcactcttaaagtgcatagagctgtgctgtggttccgtgaaggagatgagagaaagatacgccaaaatcgtcgagatacccttcaactccaccaacaagtaccagttgtctattcataagaaccccaacacatcggagccccaacacctgttggtgatgaagggcgccccagaaaggatcctagaccgttgcagctctatcctcctccacggcaaggagcagcccctggatgaggagctgaaagacgcctttcagaacgcctatttggagctggggggcctcggagaacgagtcctaggtttctgccacctctttctgccagatgaacagtttcctgaagggttccagtttgacactgacgatgtgaatttccctatcgataatctgtgctttgttgggctcatctccatgattgaccctccacgggcggccgttcctgatgccgtgggcaaatgtcgaagtgctggaattaaggtcatcatggtcacaggagaccatccaatcacagctaaagctattgccaaaggtgtgggcatcatctcagaaggcaatgagaccgtggaagacattgctgcccgcctcaacatcccagtcagccaggtgaaccccagggatgccaaggcctgcgtagtacacggcagtgatctaaaggacatgacctccgagcagctggatgacattttgaagtaccacactgagatagtgtttgccaggacctcccctcagcagaagctcatcattgtggaaggctgccaaagacagggtgctatcgtggctgtgactggtgacggtgtgaatgactctccagctttgaagaaagcagacattggggttgctatggggattgctggctcagatgtgtccaagcaagctgctgacatgattcttctggatgacaactttgcctcaattgtgactggagtagaggaaggtcgtctgatctttgataacttgaagaaatccattgcttataccttaaccagtaacattcccgagatcaccccgttcctgatatttattattgcaaacattccactaccactggggactgtcaccatcctctgcattgacttgggcactgacatggttcctgccatctccctggcttatgagcaggctgagagtgacatcatgaagagacagcccagaaatcccaaaacagacaaacttgtgaatgagcggctgatcagcatggcctatgggcagattggaatgatccaggccctgggaggcttctttacttactttgtgattctggctgagaacggcttcctcccaattcacctgttgggcctccgagtggactgggatgaccgctggatcaacgatgtggaagacagctacgggcagcagtggacctatgagcagaggaaaatcgtggagttcacctgccacacagccttcttcgtcagtatcgtggtggtgcagtgggccgacttggtcatctgtaagaccaggaggaattcggtcttccagcaggggatgaagaacaagatcttgatatttggcctctttgaagagacagccctggctgctttcctttcctactgccctggaatgggtgttgctcttaggatgtatcccctcaaacctacctggtggttctgtgccttcccctactctcttctcatcttcgtatatgacgaagtcagaaaactcatcatcaggcgacgccctggcggctgggtggagaaggaaacctactattagccccccgtcctgcacgccgtggagcatcaggccacacactctgcatccgacacccaccccctctttgtgtacttcagtcttggagtttggaactctaccctggtaggaaagcaccgcagcatgtggggaagcaagacgtcctggaatgaagcatgtagctctatggggggaggggggagggctgcctgaaaaccatccatctgtggaaatgacagcggggaaggtttttatgtgcctttttgtttttgtaaaaaaggaacacccggaaagactgaaagaatacattttatatctggatttttacaaataaagatggctattataatggaa

SEQ ID NO: 7 - protein, 30 aa, Homo sapiens
QSQWASTRGGHKERPTVMLFPFRNLGSPTQ

SEQ ID NO: 8 - protein, 369 aa, Homo sapiens
MLKPSLPFTSLLFLQLPLLGVGLNTTILTPNGNEDTTADFFLTTMPTDSLSVSTLPLPEVQCFVFNVEYMNCTWNSSSEPQPTNLTLHYWYKNSDNDKVQKCSHYLFSEEITSGCQLQKKEIHLYQTFVVQLQDPREPRRQATQMLKLQNLVIPWAPENLTLHKLSESQLELNWNNRFLNHCLEHLVQYRTDWDHSWTEQSVDYRHKFSLPSVDGQKRYTFRVRSRFNPLCGSAQHWSEWSHPIHWGSNTSKENPFLFALEAVVISVGSMGLIISLLCVYFWLERTMPRIPTLKNLEDLVTEYHGNFSAWSGVSKGLAESLQPDYSERLCLVSEIPPKGGALGEGPGASPCNQHSPYWAPPCYTLKPET

SEQ ID NO: 9 - DNA, 1480 bp, Homo sapiens
  source 1..1480: mol_type = genomic DNA
acagacagactacacccagggaatgaagagcaagcgccatgttgaagccatcattaccattcacatccctcttattcctgcagctgcccctgctgggagtggggctgaacacgacaattctgacgcccaatgggaatgaagacaccacagctgatttcttcctgaccactatgcccactgactccctcagtgtttccactctgcccctcccagaggttcagtgttttgtgttcaatgtcgagtacatgaattgcacttggaacagcagctctgagccccagcctaccaacctcactctgcattattggtacaagaactcggataatgataaagtccagaagtgcagccactatctattctctgaagaaatcacttctggctgtcagttgcaaaaaaaggagatccacctctaccaaacatttgttgttcagctccaggacccacgggaacccaggagacaggccacacagatgctaaaactgcagaatctggtgatcccctgggctccagagaacctaacacttcacaaactgagtgaatcccagctagaactgaactggaacaacagattcttgaaccactgtttggagcacttggtgcagtaccggactgactgggaccacagctggactgaacaatcagtggattatagacataagttctccttgcctagtgtggatgggcagaaacgctacacgtttcgtgttcggagccgctttaacccactctgtggaagtgctcagcattggagtgaatggagccacccaatccactgggggagcaatacttcaaaagagaatcctttcctgtttgcattggaagccgtggttatctctgttggctccatgggattgattatcagccttctctgtgtgtatttctggctggaacggacgatgccccgaattcccaccctgaagaacctagaggatcttgttactgaataccacgggaacttttcggcctggagtggtgtgtctaagggactggctgagagtctgcagccagactacagtgaacgactctgcctcgtcagtgagattcccccaaaaggaggggcccttggggaggggcctggggcctccccatgcaaccagcatagcccctactgggcccccccatgttacaccctaaagcctgaaacctgaaccccaatcctctgacagaagaaccccagggtcctgtagccctaagtggtactaactttccttcattcaacccacctgcgtctcatactcacctcaccccactgtggctgatttggaattttgtgcccccatgtaagcaccccttcatttggcattccccacttgagaattacccttttgccccgaacatgtttttcttctccctcagtctggcccttccttttcgcaggattcttcctccctccctctttccctcccttcctctttccatctaccctccgattgttcctgaaccgatgagaaataaagtttctgttgataatcatcaaaaa

SEQ ID NO: 10 - protein, 451 aa, Homo sapiens
MATPLVAGPAALRFAAAASWQVVRGRCVEHFPRVLEFLRSLRAVAPGLVRYRHHERLCMGLKAKVVVELILQGRPWAQVLKALNHHFPESGPIVRDPKATKQDLRKILEAQETFYQQVKQLSEAPVDLASKLQELEQEYGEPFLAAMEKLLFEYLCQLEKALPTPQAQQLQDVLSWMQPGVSITSSLAWRQYGVDMGWLLPECSVTDSVNLAEPMEQNPPQQQRLALHNPLPKAKPGTHLPQGPSSRTHPEPLAGRHFNLAPLGRRRVQSQWASTRGGHKERPTVMLFPFRNLGSPTQVISKPESKEEHAIYTADLAMGTRAASTGKSKSPCQTLGGRALKENPVDLPATEQKENCLDCYMDPLRLSLLPPRARKPVCPPSLCSSVITIGDLVLDSDEEENGQGEGKESLENYQKTKFDTLIPTLCEYLPPSGHGAIPVSSCDCRDSSRPL

SEQ ID NO: 11 - protein, 354 aa, Homo sapiens
MATPLVAGPAALRFAAAASWQVVRGRCVEHFPRVLEFLRSLRAVAPGLVRYRHHERLCMGLKAKVVVELILQGRPWAQVLKALNHHFPESGPIVRDPKATKQDLRKILEAQETFYQQVKQLSEAPVDLASKLQELEQEYGEPFLAAMEKLLFEYLCQLEKALPTPQAQQLQDVLSWMQPGVSITSSLAWRQYGVDMGWLLPECSVTDSVNLAEPMEQNPPQQQRLALHNPLPKAKPGTHLPQGPSSRTHPEPLAGRHFNLAPLGRRRVQSQWASTRGGHKERPTVMLFPFRNLGSPTQVISKPESKEEHAIYTADLAMGTRAASTGKSKSPCQTLGGRALKENPVDLPATEQKE

SEQ ID NO: 12 - DNA, 1801 bp, Homo sapiens
  source 1..1801: mol_type = genomic DNA
gaggcaccctcgggctcgagacagcggcgacgtttaaagctgagcgacccagtgccactggagacggtcagcttctccactcaggctcctccagcccgagccagaagaccccctcccccagaattctgggggccgatggaagggagccgagtcagatcgcgaggtacccagagccgacagaccggagcgacagggagttgccagaagccccgcccctaggagtgatcggaaagcctcacccatccgggtgaggaacccggagggaccgcctccgggcggagcccgccgaccatggctacgcccctggtggcgggtcccgcagctctacgcttcgccgccgcggctagctggcaggttgtgcgcggacgctgcgtggaacattttccgcgagtactggagtttctgcgatctctgcgcgctgttgcccctggcttggttcgctaccggcaccacgaacgcctttgtatgggcctaaaggccaaggtggtggtggagctgatcctgcagggccggccttgggcccaagtcctgaaagccctgaatcaccactttccagaatctggacctatagtgcgggatcccaaggctacaaagcaggatctgaggaagattttggaggcacaggaaactttttaccagcaggtgaagcagctgtcagaggctcctgtggatttggcctcgaagctgcaggaacttgaacaagagtatggggaaccctttctggctgccatggaaaagctgctttttgagtacttgtgtcagctggagaaagcactgcctacaccgcaggcacagcagcttcaggatgtgctgagttggatgcagcctggagtctctatcacctcttctcttgcctggagacaatatggtgtggacatggggtggctgcttccagagtgctctgttactgactcagtgaacctggctgagcccatggaacagaatcctcctcagcaacaaagactagcactccacaatcccctgccaaaagccaagcctggcacacatcttcctcagggaccatcttcaaggacgcacccagaacctctagctggccgacacttcaatctggcccctctaggccgacgaagagttcagtcccaatgggcctccactaggggaggccataaggagcgccccacagtcatgctgtttccctttaggaatctcggctcaccaacccaggtcatatctaagcctgagagcaaggaagaacatgcgatatacacagcagacctagccatgggcacaagagcagcctccactgggaagtctaagagtccatgccagaccctggggggaagggctctgaaggagaacccagttgacttgcctgccacagagcaaaaggagaattgcttggattgctacatggaccccctgagactatcattattacctcctagggccaggaagccagtgtgtcctccgtctctgtgcagctccgtcattaccataggggacttggttttagactctgatgaggaagaaaatggccagggggaaggaaaggaatctctggaaaactatcagaagacaaagtttgacaccttgatacccactctctgtgaatacctacccccttctggccacggtgccatacctgtttcttcctgtgactgtagagacagttctagacctttgtgatagaactaaaatgctctctgtactctagtctcctgcctcctcagctctgcaagtagtttagtaggaatgaagtggaagtccaggcttggattgcctaactacactgctaaaaatatttgtaatccttaataattaaactttggatttgttaaaa

SEQ ID NO: 13 - DNA, 1696 bp, Homo sapiens
  source 1..1696: mol_type = genomic DNA
gaggcaccctcgggctcgagacagcggcgacgtttaaagctgagcgacccagtgccactggagacggtcagcttctccactcaggctcctccagcccgagccagaagaccccctcccccagaattctgggggccgatggaagggagccgagtcagatcgcgaggtacccagagccgacagaccggagcgacagggagttgccagaagccccgcccctaggagtgatcggaaagcctcacccatccgggtgaggaacccggagggaccgcctccgggcggagcccgccgaccatggctacgcccctggtggcgggtcccgcagctctacgcttcgccgccgcggctagctggcaggttgtgcgcggacgctgcgtggaacattttccgcgagtactggagtttctgcgatctctgcgcgctgttgcccctggcttggttcgctaccggcaccacgaacgcctttgtatgggcctaaaggccaagacaaagcaggatctgaggaagattttggaggcacaggaaactttttaccagcaggtgaagcagctgtcagaggctcctgtggatttggcctcgaagctgcaggaacttgaacaagagtatggggaaccctttctggctgccatggaaaagctgctttttgagtacttgtgtcagctggagaaagcactgcctacaccgcaggcacagcagcttcaggatgtgctgagttggatgcagcctggagtctctatcacctcttctcttgcctggagacaatatggtgtggacatggggtggctgcttccagagtgctctgttactgactcagtgaacctggctgagcccatggaacagaatcctcctcagcaacaaagactagcactccacaatcccctgccaaaagccaagcctggcacacatcttcctcagggaccatcttcaaggacgcacccagaacctctagctggccgacacttcaatctggcccctctaggccgacgaagagttcagtcccaatgggcctccactaggggaggccataaggagcgccccacagtcatgctgtttccctttaggaatctcggctcaccaacccaggtcatatctaagcctgagagcaaggaagaacatgcgatatacacagcagacctagccatgggcacaagagcagcctccactgggaagtctaagagtccatgccagaccctggggggaagggctctgaaggagaacccagttgacttgcctgccacagagcaaaaggagaattgcttggattgctacatggaccccctgagactatcattattacctcctagggccaggaagccagtgtgtcctccgtctctgtgcagctccgtcattaccataggggacttggttttagactctgatgaggaagaaaatggccagggggaaggaaaggaatctctggaaaactatcagaagacaaagtttgacaccttgatacccactctctgtgaatacctacccccttctggccacggtgccatacctgtttcttcctgtgactgtagagacagttctagacctttgtgatagaactaaaatgctctctgtactctagtctcctgcctcctcagctctgcaagtagtttagtaggaatgaagtggaagtccaggcttggattgcctaactacactgctaaaaatatttgtaatccttaataattaaactttggatttgttaaaa

SEQ ID NO: 14 - DNA, 2182 bp, Homo sapiens
  source 1..2182: mol_type = genomic DNA
ctcttaccgcccttttccggggcaagggaagctagtagcggagccggaagtgaggcaccctcgggctcgagacagcggcgacgtttaaagctgagcgacccagtgccactggagacggtcagcttctccactcaggctcctccagcccgagccagaagaccccctcccccagaattctgggggccgatggaagggagccgagtcagatcgcgaggtacccagagccgacagaccggagcgacagggagttgccagaagccccgcccctaggagtgatcggaaagcctcacccatccgggtgaggaacccggagggaccgcctccgggcggagcccgccgaccatggctacgcccctggtggcgggtcccgcagctctacgcttcgccgccgcggctagctggcaggttgtgcgcggacgctgcgtggaacattttccgcgagtactggagtttctgcgatctctgcgcgctgttgcccctggcttggttcgctaccggcaccacgaacgcctttgtatgggcctaaaggccaaggtggtggtggagctgatcctgcagggccggccttgggcccaagtcctgaaagccctgaatcaccactttccagaatctggacctatagtgcgggatcccaaggctacaaagcaggatctgaggaagattttggaggcacaggaaactttttaccagcaggtgaagcagctgtcagaggctcctgtggatttggcctcgaagctgcaggaacttgaacaagagtatggggaaccctttctggctgccatggaaaagctgctttttgagtacttgtgtcagctggagaaagcactgcctacaccgcaggcacagcagcttcaggatgtgctgagttggatgcagcctggagtctctatcacctcttctcttgcctggagacaatatggtgtggacatggggtggctgcttccagagtgctctgttactgactcagtgaacctggctgagcccatggaacagaatcctcctcagcaacaaagactagcactccacaatcccctgccaaaagccaagcctggcacacatcttcctcagggaccatcttcaaggacgcacccagaacctctagctggccgacacttcaatctggcccctctaggccgacgaagagttcagtcccaatgggcctccactaggggaggccataaggagcgccccacagtcatgctgtttccctttaggaatctcggctcaccaacccaggtcatatctaagcctgagagcaaggaagaacatgcgatatacacagcagacctagccatgggcacaagagcagcctccactgggaagtctaagagtccatgccagaccctggggggaagggctctgaaggagaacccagttgacttgcctgccacagagcaaaaggagtgagtggaacagagttgcttcttactaggagcacattctttgcctgccttcccttcatcctatcctctttgcttgctctcacctcaggaattgcttggattgctacatggaccccctgagactatcattattacctcctagggccaggaagccaggtaggtagtctgagtcaggattggatcaacagcctcctctcttggggactctcaagagcctgtgttcatctagaagtagtagtttgattctggtttccctcctacagtgtgtcctccgtctctgtgcagctccgtcattaccataggggacttggttttagactctgatgaggaagaaaatggccagggggaaggaaaggtgagtgggaaggagcagaaagctgggaaaggggatgggtagaacaagactgagaaatccacatgcttcagaattcagagggttcagggaatggtttcggatagtaggctctccctgctcccttctctacaggaatctctggaaaactatcagaagacaaagtttgacaccttgatacccactctctgtgaatacctacccccttctggccacggtgccatacctgtttcttcctgtgactgtagagacagttctagacctttgtgatagaactaaaatgctctctgtactctagtctcctgcctcctcagctctgcaagtagtttagtaggaatgaagtggaagtccaggcttggattgcctaactacactgctaaaaatatttgtaatccttaataattaaactttggatttgttaaaatac

SEQ ID NO: 15 - protein, 112 aa, Simian immunodeficiency virus
MSDPRERIPPGNSGEETIEEAFEWLNRTVEGINRAAVNHLPRELIFQVWQRSWEYWHDEMGMSESYTKYRYLCLIQKALFMHCKKGCRCLGEGHGAGGWRTGPPPPPPPGLA

SEQ ID NO: 16 - protein, 113 aa, Human immunodeficiency virus 2
MTDPRERVPPGNSGEETIGEAFEWLERTIEALNREAVNHLPRELIFQVWQRSWRYWHDEQGMSASYTKYRYLCLMQKAIFTHFKRGCTCWGEDMGREGLEDQGPPPPPPPGLV

SEQ ID NO: 17 - DNA, 339 bp, Simian immunodeficiency virus
  source 1..339: mol_type = other DNA
atgtcagatcccagggagagaatcccacctggaaacagtggagaagagacaataggagaggccttcgaatggctaaacagaacagtagaggagataaacagagaggcagtaaaccacctaccaagggagctgattttccaggtttggcaaaggtcttgggaatactggcatgatgaacaagggatgtcacaaagctatgtaaaatacagatacttgtgtttaatgcaaaaggctttatttatgcattgcaagaaaggctgtagatgtctaggggaaggacacggggcaggaggatggagaccaggacctcctcctcctccccctccaggactagcatga

SEQ ID NO: 18 - DNA, 342 bp, Human immunodeficiency virus 2
  source 1..342: mol_type = other DNA
atgacagaccccagagaaagggtaccgccaggaaacagtggagaagagaccattggagaggccttcgagtggctagagaggaccatagaagccttaaacagggaggcagtgaaccatctgccccgagagctcattttccaggtgtggcaaaggtcctggagatattggcatgatgaacaagggatgtcagcaagctacacaaagtatagatatttgtgcctaatgcaaaaagctatatttacacatttcaagagagggtgcacttgctggggggaggacatgggccgggaaggattggaagaccaaggacctccccctcctccccctccaggtctagtctaa

SEQ ID NO: 19 - RNA, 37 nt, synthetic construct
  source 1..37: mol_type = other RNA
cgcggttctatctagttacgcgttaaaccaactagaa

SEQ ID NO: 20 - RNA, 76 nt, synthetic construct
  source 1..76: mol_type = other RNA
gttttagagctagaaatagcaagttaaaataaggctagtccgttatcaacttgaaaaagtggcaccgagtcggtgc

SEQ ID NO: 21 - RNA, 86 nt, synthetic construct
  source 1..86: mol_type = other RNA
gtttaagagctatgctggaaacagcatagcaagtttaaataaggctagtccgttatcaacttgaaaaagtggcaccgagtcggtgc

SEQ ID NO: 22 - RNA, 20 nt, synthetic construct
  source 1..20: mol_type = other RNA
gtccactaggggaggccata

SEQ ID NO: 23 - RNA, 20 nt, synthetic construct
  source 1..20: mol_type = other RNA
gtcccaatgggcctccacta

SEQ ID NO: 24 - RNA, 20 nt, synthetic construct
  source 1..20: mol_type = other RNA
gacgaagagttcagtcccaa

SEQ ID NO: 25 - RNA, 20 nt, synthetic construct
  source 1..20: mol_type = other RNA
gacttcaatctggcccctct

SEQ ID NO: 26 - RNA, 20 nt, synthetic construct
  source 1..20: mol_type = other RNA
ggtgagccgagattcctaaa

SEQ ID NO: 27 - RNA, 20 nt, synthetic construct
  source 1..20: mol_type = other RNA
ggcttagatatgacctgggt

SEQ ID NO: 28 - RNA, 48 nt, synthetic construct
  source 1..48: mol_type = other RNA
gaatgggaagagcatcacggtaggccgttccttgtggcctcccctagt

SEQ ID NO: 29 - RNA, 60 nt, synthetic construct
  source 1..60: mol_type = other RNA
gaatgggaagagcatcacggtaggccgttccttgtgtccgcctctggtggaggcccattg

SEQ ID NO: 30 - RNA, 73 nt, synthetic construct
  source 1..73: mol_type = other RNA
gaatgggaagagcatcacggtaggccgttccttgtgtccgcctctggtgctagcccactgggactgaactctt

SEQ ID NO: 31 - RNA, 98 nt, synthetic construct
  source 1..98: mol_type = other RNA
gaatgggaagagcatcacggtaggccgttccttgtgtccgcctctggtgctagcccactggctctgcactctccgtcttcccaatggggccagattga

SEQ ID NO: 32 - RNA, 47 nt, synthetic construct
  source 1..47: mol_type = other RNA
gttccttgtgtccgcctctggtgctagcccactgggactgaactctt

SEQ ID NO: 33 - RNA, 72 nt, synthetic construct
  source 1..72: mol_type = other RNA
gttccttgtgtccgcctctggtgctagcccactggctctgcactctccgtcttcccaatggggccagattga

SEQ ID NO: 34 - RNA, 38 nt, synthetic construct
  source 1..38: mol_type = other RNA
gcctaccgtgatgctcttcccattcaggaatctcggct

SEQ ID NO: 35 - RNA, 59 nt, synthetic construct
  source 1..59: mol_type = other RNA
gcctaccgtgatgctcttcccattccgcaacctgggaagccctacacaggtcatatcta

SEQ ID NO: 36 - RNA, 67 nt, synthetic construct
  source 1..67: mol_type = other RNA
gctagcaccagaggcggacacaaggaacggcctaccgtgatgctcttcccattcaggaatctcggct

SEQ ID NO: 37 - RNA, 88 nt, synthetic construct
  source 1..88: mol_type = other RNA
gctagcaccagaggcggacacaaggaacggcctaccgtgatgctcttcccattccgcaacctgggaagccctacacaggtcatatcta

SEQ ID NO: 38 - RNA, 38 nt, synthetic construct
  source 1..38: mol_type = other RNA
gctctgcactctccgtcttcccaatggggccagattga

SEQ ID NO: 39 - RNA, 33 nt, synthetic construct
  source 1..33: mol_type = other RNA
gcactctccgtcttcccaatggggccagattga

SEQ ID NO: 40 - RNA, 27 nt, synthetic construct
  source 1..27: mol_type = other RNA
tccgtcttcccaatggggccagattga

SEQ ID NO: 41 - RNA, 24 nt, synthetic construct
  source 1..24: mol_type = other RNA
gtcttcccaatggggccagattga

SEQ ID NO: 42 - RNA, 23 nt, synthetic construct
  source 1..23: mol_type = other RNA
tcttcccaatggggccagattga

SEQ ID NO: 43 - RNA, 21 nt, synthetic construct
  source 1..21: mol_type = other RNA
ttcccaatggggccagattga

SEQ ID NO: 44 - RNA, 38 nt, synthetic construct
  source 1..38: mol_type = other RNA
attccgcaacctgggaagccctacacaggtcatatcta

SEQ ID NO: 45 - RNA, 33 nt, synthetic construct
  source 1..33: mol_type = other RNA
gcaacctgggaagccctacacaggtcatatcta

SEQ ID NO: 46 - RNA, 27 nt, synthetic construct
  source 1..27: mol_type = other RNA
tgggaagccctacacaggtcatatcta

SEQ ID NO: 47 - RNA, 24 nt, synthetic construct
  source 1..24: mol_type = other RNA
gaagccctacacaggtcatatcta

SEQ ID NO: 48 - RNA, 23 nt, synthetic construct
  source 1..23: mol_type = other RNA
aagccctacacaggtcatatcta

SEQ ID NO: 49 - RNA, 21 nt, synthetic construct
  source 1..21: mol_type = other RNA
gccctacacaggtcatatcta

SEQ ID NO: 50 - DNA, 81 bp, synthetic construct
  source 1..81: mol_type = other DNA
cagtgggctagcaccagaggcggacacaaggaacggcctaccgtgatgctcttcccattccgcaacctgggaagccctaca

SEQ ID NO: 51 - DNA, 81 bp, synthetic construct
  source 1..81: mol_type = other DNA
gggaagagcatcacggtaggccgttccttgtgtccgcctctggtgctagcccactggctctgcactctccgtcttcccaat

SEQ ID NO: 52 - DNA, 81 bp, synthetic construct; identical to SEQ ID NO: 50
  source 1..81: mol_type = other DNA
  modified_base 78: mod_base = OTHER; note = Nucleotide with phosphorothioate linkage
  modified_base 79: mod_base = OTHER; note = Nucleotide with phosphorothioate linkage
  modified_base 80: mod_base = OTHER; note = Nucleotide with phosphorothioate linkage

SEQ ID NO: 53 - DNA, 81 bp, synthetic construct; identical to SEQ ID NO: 51
  source 1..81: mol_type = other DNA
  modified_base 78: mod_base = OTHER; note = Nucleotide with phosphorothioate linkage
  modified_base 79: mod_base = OTHER; note = Nucleotide with phosphorothioate linkage
  modified_base 80: mod_base = OTHER; note = Nucleotide with phosphorothioate linkage

SEQ ID NO: 54 - DNA, 73 bp, synthetic construct
  source 1..73: mol_type = other DNA
tagcaccagaggcggacacaaggaacggcctaccgtgatgctcttcccattccgcaacctgggaagccctaca

SEQ ID NO: 55 - DNA, 73 bp, synthetic construct
  source 1..73: mol_type = other DNA
catcacggtaggccgttccttgtgtccgcctctggtgctagcccactggctctgcactctccgtcttcccaat

SEQ ID NO: 56 - DNA, 63 bp, synthetic construct
  source 1..63: mol_type = other DNA
ggcggacacaaggaacggcctaccgtgatgctcttcccattccgcaacctgggaagccctaca

SEQ ID NO: 57 - DNA, 63 bp, synthetic construct
  source 1..63: mol_type = other DNA
ggccgttccttgtgtccgcctctggtgctagcccactggctctgcactctccgtcttcccaat

SEQ ID NO: 58 - DNA, 95 bp, synthetic construct
  source 1..95: mol_type = other DNA
ggagagtgcagagccagtgggctagcaccagaggcggacacaaggaacggcctaccgtgatgctcttcccattccgcaacctgggaagccctaca

SEQ ID NO: 59 - DNA, 95 bp, synthetic construct
  source 1..95: mol_type = other DNA
ccaggttgcggaatgggaagagcatcacggtaggccgttccttgtgtccgcctctggtgctagcccactggctctgcactctccgtcttcccaat

SEQ ID NO: 60 - DNA, 109 bp, synthetic construct
  source 1..109: mol_type = other DNA
  modified_base 106: mod_base = OTHER; note = Nucleotide with phosphorothioate linkage
  modified_base 107: mod_base = OTHER; note = Nucleotide with phosphorothioate linkage
  modified_base 108: mod_base = OTHER; note = Nucleotide with phosphorothioate linkage
gcatatacgatacaaggctgttagagagataattagaattaatttgactgtaaacacaaagatattagtacaaaatacgtgacgtagaaagtaataatttcttgggtag

SEQ ID NO: 61 - DNA, 121 bp, synthetic construct
  source 1..121: mol_type = other DNA
  modified_base 118: mod_base = OTHER; note = Nucleotide with phosphorothioate linkage
  modified_base 119: mod_base = OTHER; note = Nucleotide with phosphorothioate linkage
  modified_base 120: mod_base = OTHER; note = Nucleotide with phosphorothioate linkage
ctctctaacagccttgtatcgtatatgcaaatatgaaggaatcatgggaaataggccctctgttgtgacactcaccatgtcagtgcccagatcgatacacaaaattgtcacgttgcccaaa

SEQ ID NO: 62 - DNA, 199 bp, synthetic construct
  source 1..199: mol_type = other DNA
  misc_difference 158: note = a, c, t, g, unknown or other
  misc_difference 159: note = a, c, t, g, unknown or other
  misc_difference 160: note = a, c, t, g, unknown or other
  misc_difference 161: note = a, c, t, g, unknown or other
  misc_difference 162: note = a, c, t, g, unknown or other
  misc_difference 163: note = a, c, t, g, unknown or other
  misc_difference 164: note = a, c, t, g, unknown or other
  misc_difference 165: note = a, c, t, g, unknown or other
  misc_difference 166: note = a, c, t, g, unknown or other
  misc_difference 167: note = a, c, t, g, unknown or other
  modified_base 196: mod_base = OTHER; note = Nucleotide with phosphorothioate linkage
  modified_base 197: mod_base = OTHER; note = Nucleotide with phosphorothioate linkage
  modified_base 198: mod_base = OTHER; note = Nucleotide with phosphorothioate linkage
atatatcttgtggaaaggacgaaacaccgagtagcgcgagcacagctagttttagagctagaaatagcaagttaaaataaggctagtccgttatcaacttgaaaaagtggcaccgagtcggtgctccgtggggtgagctgtgctcgcgcttttttttnnnnnnnnnngctagcatcagagttggacacatggaacgttg

SEQ ID NO: 63 - DNA, 140 bp, synthetic construct
  source 1..140: mol_type = other DNA
  modified_base 137: mod_base = OTHER; note = Nucleotide with phosphorothioate linkage
  modified_base 138: mod_base = OTHER; note = Nucleotide with phosphorothioate linkage
  modified_base 139: mod_base = OTHER; note = Nucleotide with phosphorothioate linkage
ggtgtttcgtcctttccacaagatatataaagccaagaaatcgaaatactttcaagttacggtaagcatatgatagtccattttaaaacataattttaaaactgcaaactacccaagaaattattactttctacgtcacg

SEQ ID NO: 64 - RNA, 20 nt, synthetic construct
  source 1..20: mol_type = other RNA
gcaaacattccactaccact

SEQ ID NO: 65 - RNA, 20 nt, synthetic construct
  source 1..20: mol_type = other RNA
gcgccagcctcatggatgct

SEQ ID NO: 66 - RNA, 20 nt, synthetic construct
  source 1..20: mol_type = other RNA
gtttcccaacgccagcctca

SEQ ID NO: 67 - RNA, 19 nt, synthetic construct
  source 1..19: mol_type = other RNA
gcctgggttggggcaaaga

SEQ ID NO: 68 - RNA, 20 nt, synthetic construct
  source 1..20: mol_type = other RNA
gcttgtccatcactggcatc

SEQ ID NO: 69 - RNA, 20 nt, synthetic construct
  source 1..20: mol_type = other RNA
gccccgcccttgtccatcac

SEQ ID NO: 70 - RNA, 21 nt, synthetic construct
  source 1..21: mol_type = other RNA
gtcagggttctggatatctgt

SEQ ID NO: 71 - RNA, 19 nt, synthetic construct
  source 1..19: mol_type = other RNA
gagtctctcagctggtaca

SEQ ID NO: 72 - RNA, 20 nt, synthetic construct
  source 1..20: mol_type = other RNA
gggtagtgggtgagggaccc

SEQ ID NO: 73 - RNA, 20 nt, synthetic construct
  source 1..20: mol_type = other RNA
gacacagacagactacaccc

SEQ ID NO: 74 - RNA, 19 nt, synthetic construct
  source 1..19: mol_type = other RNA
ggtaatgatggcttcaaca

SEQ ID NO: 75 - RNA, 19 nt, synthetic construct
  source 1..19: mol_type = other RNA
ggaataagagggatgtgaa

SEQ ID NO: 76 - RNA, 20 nt, synthetic construct
  source 1..20: mol_type = other RNA
gggcagctgcaggaataaga

SEQ ID NO: 77 - RNA, 20 nt, synthetic construct
  source 1..20: mol_type = other RNA
gttcagccccactcccagca

SEQ ID NO: 78 - RNA, 20 nt, synthetic construct
  source 1..20: mol_type = other RNA
gccagatttcccaccagctg

SEQ ID NO: 79 - RNA, 20 nt, synthetic construct
  source 1..20: mol_type = other RNA
ggggccactagggacaggat

SEQ ID NO: 80 - RNA, 20 nt, synthetic construct
  source 1..20: mol_type = other RNA
gatggagccagagaggatcc

SEQ ID NO: 81 - RNA, 20 nt, synthetic construct
  source 1..20: mol_type = other RNA
gcagctcaggttctgggaga

SEQ ID NO: 82 - RNA, 20 nt, synthetic construct
  source 1..20: mol_type = other RNA
gatcagtgaaacgcaccaga

SEQ ID NO: 83 - RNA, 38 nt, synthetic construct
  source 1..38: mol_type = other RNA
acacaaaattgtcacgttgcccaaaggtagtggaatgt

SEQ ID NO: 84 - RNA, 45 nt, synthetic construct
  source 1..45: mol_type = other RNA
gatcgatacacaaaattgtcacgttgcccaaaggtagtggaatgt

SEQ ID NO: 85 - RNA, 45 nt, synthetic construct
  source 1..45: mol_type = other RNA
gctagcatcagagttggacacatggaacgttgatccatgaggctg

SEQ ID NO: 86 - RNA, 43 nt, synthetic construct
  source 1..43: mol_type = other RNA
gctagcatcagagttggacacatggaacgttgggctggcgttg

SEQ ID NO: 87 - RNA, 43 nt, synthetic construct
  source 1..43: mol_type = other RNA
gctggtcattgcggtctcattggtgtacggtattgccccaacc

SEQ ID NO: 88 - RNA, 36 nt, synthetic construct
  source 1..36: mol_type = other RNA
gttccggtctcattggtgtacggtattgccccaacc

SEQ ID NO: 89 - RNA, 44 nt, synthetic construct
  source 1..44: mol_type = other RNA
gctggtcattgcggtctcattggtgtacggtagccagtgatgga

SEQ ID NO: 90 - RNA, 37 nt, synthetic construct
  source 1..37: mol_type = other RNA
gttccggtctcattggtgtacggtagccagtgatgga

SEQ ID NO: 91 - RNA, 44 nt, synthetic construct
  source 1..44: mol_type = other RNA
gctggtcattgcggtctcattggtgtacggtaatggacaagggc

SEQ ID NO: 92 - RNA, 37 nt, synthetic construct
  source 1..37: mol_type = other RNA
gttccggtctcattggtgtacggtaatggacaagggc

SEQ ID NO: 93 - RNA, 45 nt, synthetic construct
  source 1..45: mol_type = other RNA
gctagcatcagagttggacacatggaacgttggatatccagaacc

SEQ ID NO: 94 - RNA, 38 nt, synthetic construct
  source 1..38: mol_type = other RNA
gcctaccgtgatgctcttcccattcgatatccagaacc

SEQ ID NO: 95 - RNA, 44 nt, synthetic construct
  source 1..44: mol_type = other RNA
gctagcatcagagttggacacatggaacgttgaccagctgagag

SEQ ID NO: 96 - RNA, 37 nt, synthetic construct
  source 1..37: mol_type = other RNA
gcctaccgtgatgctcttcccattcaccagctgagag

SEQ ID NO: 97 - RNA, 43 nt, synthetic construct
  source 1..43: mol_type = other RNA
gctggtctatgcggtctctaaggtgtacggtatccctcaccca

SEQ ID NO: 98 - RNA, 45 nt, synthetic construct
  source 1..45: mol_type = other RNA
gctggtctatgcggtctctaaggtgtacggtatgtagtctgtctg

SEQ ID NO: 99 - RNA, 45 nt, synthetic construct
  source 1..45: mol_type = other RNA
gctagcatcagagttggacacatggaacgttgtgaagccatcatt

SEQ ID NO: 100 - RNA, 46 nt, synthetic construct
  source 1..46: mol_type = other RNA
gctagcatcagagttggacacatggaacgttgacatccctcttatt

SEQ ID NO: 101 - RNA, 45 nt, synthetic construct
  source 1..45: mol_type = other RNA
gctagcatcagagttggacacatggaacgttgtattcctgcagct

SEQ ID NO: 102 - RNA, 43 nt, synthetic construct
  source 1..43: mol_type = other RNA
gctagcatcagagttggacacatggaacgttgtgggagtgggg

SEQ ID NO: 103 - RNA, 44 nt, synthetic construct
  source 1..44: mol_type = other RNA
gctagcatcagagttggacacatggaacgttgctggtgggaaat

SEQ ID NO: 104 - RNA, 44 nt, synthetic construct
  source 1..44: mol_type = other RNA
gctggtcattgcggtctcattggtgtacggtactgtccctagtg

SEQ ID NO: 105 - RNA, 37 nt, synthetic construct
  source 1..37: mol_type = other RNA
gttccggtctcattggtgtacggtactgtccctagtg

SEQ ID NO: 106 - RNA, 43 nt, synthetic construct
  source 1..43: mol_type = other RNA
gctggtcattgcggtctcattggtgtacggtatcctctctggc

SEQ ID NO: 107 - RNA, 36 nt, synthetic construct
  source 1..36: mol_type = other RNA
gttccggtctcattggtgtacggtatcctctctggc

SEQ ID NO: 108 - RNA, 41 nt, synthetic construct
  source 1..41: mol_type = other RNA
gctagcatcagagttggacacatggaacgttgcccagaacc

SEQ ID NO: 109 - RNA, 34 nt, synthetic construct
  source 1..34: mol_type = other RNA
gcctaccgtgatgctcttcccattccccagaacc

SEQ ID NO: 110 - RNA, 45 nt, synthetic construct
  source 1..45: mol_type = other RNA
gctagcatcagagttggacacatggaacgttgggtgcgtttcact

SEQ ID NO: 111 - RNA, 38 nt, synthetic construct
  source 1..38: mol_type = other RNA
gcctaccgtgatgctcttcccattcggtgcgtttcact

SEQ ID NO: 112 - DNA, 1121 bp, synthetic construct
  source 1..1121: mol_type = other DNA
  modified_base 33: mod_base = OTHER; note = Nucleotide with phosphorothioate linkage
  modified_base 34: mod_base = OTHER; note = Nucleotide with phosphorothioate linkage
  modified_base 35: mod_base = OTHER; note = Nucleotide with phosphorothioate linkage
  modified_base 36: mod_base = OTHER; note = Nucleotide with phosphorothioate linkage
  modified_base 37: mod_base = OTHER; note = Nucleotide with phosphorothioate linkage
  modified_base 1084: mod_base = OTHER; note = Nucleotide with phosphorothioate linkage
  modified_base 1085: mod_base = OTHER; note = Nucleotide with phosphorothioate linkage
  modified_base 1086: mod_base = OTHER; note = Nucleotide with phosphorothioate linkage
  modified_base 1087: mod_base = OTHER; note = Nucleotide with phosphorothioate linkage
  modified_base 1088: mod_base = OTHER; note = Nucleotide with phosphorothioate linkage
taccgtacaccaatgagaccgcaatgaccagctgctgacctcttctcttcctcccacagatatcggatccggcgccacaaatttcagcctgctgaaacaggccggcgacgtggaagagaaccccggcccttccatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtgataacctgcaggctgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatgggctagcatcagagttggacacatggaacgttg

SEQ ID NO: 113 - DNA, 1107 bp, synthetic construct
  source 1..1107: mol_type = other DNA
  modified_base 26: mod_base = OTHER; note = Nucleotide with phosphorothioate linkage
  modified_base 27: mod_base = OTHER; note = Nucleotide with phosphorothioate linkage
  modified_base 28: mod_base = OTHER; note = Nucleotide with phosphorothioate linkage
  modified_base 29: mod_base = OTHER; note = Nucleotide with phosphorothioate linkage
  modified_base 30: mod_base = OTHER; note = Nucleotide with phosphorothioate linkage
  modified_base 1077: mod_base = OTHER; note = Nucleotide with phosphorothioate linkage
  modified_base 1078: mod_base = OTHER; note = Nucleotide with phosphorothioate linkage
  modified_base 1079: mod_base = OTHER; note = Nucleotide with phosphorothioate linkage
  modified_base 1080: mod_base = OTHER; note = Nucleotide with phosphorothioate linkage
  modified_base 1081: mod_base = OTHER; note = Nucleotide with phosphorothioate linkage
taccgtacaccaatgagaccggaactgctgacctcttctcttcctcccacagatatcggatccggcgccacaaatttcagcctgctgaaacaggccggcgacgtggaagagaaccccggcccttccatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtgataacctgcaggctgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatgggcctaccgtgatgctcttcccattc

SEQ ID NO: 114 - DNA, 1023 bp, synthetic construct
  source 1..1023: mol_type = other DNA
  modified_base 33: mod_base = OTHER; note = Nucleotide with phosphorothioate linkage
  modified_base 34: mod_base = OTHER; note = Nucleotide with phosphorothioate linkage
  modified_base 35: mod_base = OTHER; note = Nucleotide with phosphorothioate linkage
  modified_base 36: mod_base = OTHER; note = Nucleotide with phosphorothioate linkage
  modified_base 37: mod_base = OTHER; note = Nucleotide with phosphorothioate linkage
  modified_base 986: mod_base = OTHER; note = Nucleotide with phosphorothioate linkage
  modified_base 987: mod_base = OTHER; note = Nucleotide with phosphorothioate linkage
  modified_base 988: mod_base = OTHER; note = Nucleotide with phosphorothioate linkage
  modified_base 989: mod_base = OTHER; note = Nucleotide with phosphorothioate linkage
  modified_base 990: mod_base = OTHER; note = Nucleotide with phosphorothioate linkage
taccgtacaccttagagaccgcatagaccagcgccaccatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtgacctgcaggctgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatgggctagcatcagagttggacacatggaacgttg

SEQ ID NO: 115 - DNA, 1113 bp, synthetic construct
  source 1..1113: mol_type = other DNA
  modified_base 33: mod_base = OTHER; note = Nucleotide with phosphorothioate linkage
  modified_base 34: mod_base = OTHER; note = Nucleotide with phosphorothioate linkage
  modified_base 35: mod_base = OTHER; note = Nucleotide with phosphorothioate linkage
  modified_base 36: mod_base = OTHER; note = Nucleotide with phosphorothioate linkage
  modified_base 37: mod_base = OTHER; note = Nucleotide with phosphorothioate linkage
  modified_base 1076: mod_base = OTHER; note = Nucleotide with phosphorothioate linkage
  modified_base 1077: mod_base = OTHER; note = Nucleotide with phosphorothioate linkage
  modified_base 1078: mod_base = OTHER; note = Nucleotide with phosphorothioate linkage
  modified_base 1079: mod_base = OTHER; note = Nucleotide with phosphorothioate linkage
  modified_base 1080: mod_base = OTHER; note = Nucleotide with phosphorothioate linkage
taccgtacaccaatgagaccgcaatgaccagctgctgacctcttctcttcctcccacagggatccggcgccacaaatttcagcctgctgaaacaggccggcgacgtggaagagaaccccggcccttccatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtgacctgcaggctgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatgggctagcatcagagttggacacatggaacgttg

SEQ ID NO: 116 - DNA, 1099 bp, synthetic construct
  source 1..1099: mol_type = other DNA
  modified_base 26: mod_base = OTHER; note = Nucleotide with phosphorothioate linkage
  modified_base 27: mod_base = OTHER; note = Nucleotide with phosphorothioate linkage
  modified_base 28: mod_base = OTHER; note = Nucleotide with phosphorothioate linkage
  modified_base 29: mod_base = OTHER; note = Nucleotide with phosphorothioate linkage
  modified_base 30: mod_base = OTHER; note = Nucleotide with phosphorothioate linkage
  modified_base 1069: mod_base = OTHER; note = Nucleotide with phosphorothioate linkage
  modified_base 1070: mod_base = OTHER; note = Nucleotide with phosphorothioate linkage
  modified_base 1071: mod_base = OTHER; note = Nucleotide with phosphorothioate linkage
  modified_base 1072: mod_base = OTHER; note = Nucleotide with phosphorothioate linkage
  modified_base 1073: mod_base = OTHER; note = Nucleotide with phosphorothioate linkage
taccgtacaccaatgagaccggaactgctgacctcttctcttcctcccacagggatccggcgccacaaatttcagcctgctgaaacaggccggcgacgtggaagagaaccccggcccttccatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtgacctgcaggctgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatgggcctaccgtgatgctcttcccattc

SEQ ID NO: 117 - DNA, 805 bp, synthetic construct
  source 1..805: mol_type = other DNA
ctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtgacctgcaggctgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatgggctagcatcagagttggacacatggaacgttg

SEQ ID NO: 118 - DNA, 605 bp, synthetic construct
  source 1..605: mol_type = other DNA
gggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtgacctgcaggctgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatgggctagcatcagagttggacacatggaacgttg

SEQ ID NO: 119 - DNA, 808 bp, synthetic construct
  source 1..808: mol_type = other DNA
gcggtcacgaactccagcaggaccatgtgatcgcgcttctcgttggggtctttgctcagggcggactgggtgctcaggtagtggttgtcgggcagcagcacggggccgtcgccgatgggggtgttctgctggtagtggtcggcgagctgcacgctgccgtcctcgatgttgtggcggatcttgaagttcaccttgatgccgttcttctgcttgtcggccatgatatagacgttgtggctgttgtagttgtactccagcttgtgccccaggatgttgccgtcctccttgaagtcgatgcccttcagctcgatgcggttcaccagggtgtcgccctcgaacttcacctcggcgcgggtcttgtagttgccgtcgtccttgaagaagatggtgcgctcctggacgtagccttcgggcatggcggacttgaagaagtcgtgctgcttcatgtggtcggggtagcggctgaagcactgcacgccgtaggtcagggtggtcacgagggtgggccagggcacgggcagcttgccggtggtgcagatgaacttcagggtcagcttgccgtaggtggcatcgccctcgccctcgccggacacgctgaacttgtggccgtttacgtcgccgtccagctcgaccaggatgggcaccaccccggtgaacagctcctcgcccttgctcaccatggaagggccggggttctcttccacgtcgccggcctgtttcagcaggctgaaatttgtggcgccggatccctgtgggaggaagagaagaggtcagcagctggtcattgcggtctcattggtgtacggta

SEQ ID NO: 120 - DNA, 608 bp, synthetic construct
  source 1..608: mol_type = other DNA
gttcttctgcttgtcggccatgatatagacgttgtggctgttgtagttgtactccagcttgtgccccaggatgttgccgtcctccttgaagtcgatgcccttcagctcgatgcggttcaccagggtgtcgccctcgaacttcacctcggcgcgggtcttgtagttgccgtcgtccttgaagaagatggtgcgctcctggacgtagccttcgggcatggcggacttgaagaagtcgtgctgcttcatgtggtcggggtagcggctgaagcactgcacgccgtaggtcagggtggtcacgagggtgggccagggcacgggcagcttgccggtggtgcagatgaacttcagggtcagcttgccgtaggtggcatcgccctcgccctcgccggacacgctgaacttgtggccgtttacgtcgccgtccagctcgaccaggatgggcaccaccccggtgaacagctcctcgcccttgctcaccatggaagggccggggttctcttccacgtcgccggcctgtttcagcaggctgaaatttgtggcgccggatccctgtgggaggaagagaagaggtcagcagctggtcattgcggtctcattggtgtacggta

(19) United States
(12) Patent Application Publication
(10) Pub. No.: US 2026/0159822 A1
(43) Pub. Date: Jun. 11, 2026

(54) SYSTEMS AND METHODS FOR MODIFYING A POLYNUCLEOTIDE

(71) Applicant: The Children's Medical Center Corporation, Boston, MA (US)

(72) Inventors: Daniel E. Bauer, Boston, MA (US); Sébastien Levesque, Boston, MA (US)

(73) Assignee: The Children's Medical Center Corporation, Boston, MA (US)

(21) Appl. No.: 19/537,061

(22) Filed: Feb. 11, 2026

Related U.S. Application Data
(63) Continuation of application No. PCT/US2024/042480, filed on Aug. 15, 2024.
(60) Provisional application No. 63/532,828, filed on Aug. 15, 2023.

(51) Int. Cl. C12N 9/22; A61K 31/7105; A61K 38/45; A61P 7/00; C12N 9/12; C12N 15/113

(52) U.S. Cl. CPC C12N 9/226; A61K 31/7105; A61K 38/45; A61P 7/00; C12N 9/1276; C12N 15/1137; C12N 15/1138; C12Y 207/07049; C12N 2310/20

(57) ABSTRACT

The present disclosure features systems and methods for modifying a target polynucleotide. The present disclosure includes methods for integrating polynucleotides greater than 100 base pairs in length into a gene using prime editing guide RNAs (pegRNAs) that target a gene of interest. The methods disclosed herein include the use of pegRNAs which feature reverse transcriptase templates that are less than 100% identical in sequence to the target polynucleotide.

### CROSS REFERENCE TO RELATED APPLICATIONS

The present application is a continuation under 35 U.S.C. § 111(a) of PCT International Patent Application No. PCT/US2024/042480, filed Aug. 15, 2024, designating the United States and published in English, which claims priority to and the benefit of U.S. App. No. 63/532,828, filed Aug. 15, 2023, which is hereby incorporated by reference in its entirety.

### SEQUENCE LISTING

The instant application contains a Sequence Listing which has been submitted electronically as an XML file and is hereby incorporated by reference in its entirety. The Sequence Listing, created on Aug. 23, 2024, is named “167705-033801PCT-SL.xml” and is 182,548 bytes in size.

### BACKGROUND OF THE INVENTION

Prime editing is a genome editing technology that provides for the installation of small genetic changes, such as point mutations or small insertions and deletions, in living cells. This technology provides for the replacement or the integration of polynucleotides that are up to about 100 base pairs in length. There is a need for improved genome editing technologies that can efficiently integrate polynucleotides greater than 100 base pairs in length.

### SUMMARY OF THE INVENTION

As described below, the present disclosure features systems, compositions, and methods for improved gene editing utilizing paired prime editing and prime assembly. In particular, the present disclosure provides methods for targeted integration of long (e.g., 250, 500, 1,000, 1,500, 2,000, 2,500, 3,000 nucleotides or more) donor polynucleotides involving the use of a pair of prime editing guide RNAs (pegRNAs), each comprising a codon optimized reverse transcriptase template that is not 100% identical to a target gene, and a prime editor comprising a nucleic acid programmable DNA binding protein comprising a nickase domain fused to or complexed with a reverse transcriptase domain.

In one aspect, the present disclosure provides a genome editing system. The genome editing system includes: i) a nucleic acid programmable DNA binding protein having DNA nickase activity; ii) a reverse transcriptase; iii) a pair of prime editing guide RNA (pegRNA), where each member of the pair includes a spacer sequence complementary to a target polynucleotide sequence, a reverse transcriptase template, where the reverse transcriptase template has less than about 85% identity to the target polynucleotide sequence, and a primer binding sequence including a region complementary to a region upstream of a nick site in the double-stranded target DNA sequence; and iv) a donor sequence donor sequence comprising a 3′ overhang that is identical or non-identical to the reverse transcriptase template.

In another aspect, the present disclosure provides a method for editing a target polynucleotide. The method involves contacting the genome editing systems of any of the above aspects, or embodiments thereof, with a target polynucleotide, thereby editing the target polynucleotide.

In another aspect, the present disclosure provides a method of editing a TINF2 polynucleotide in a cell. The method involves contacting the cell with a genome editing system including: i) a nucleic acid programmable DNA binding protein having DNA nickase activity; ii) a reverse transcriptase; iii) a pair of prime editing guide RNA (pegRNA), each including a spacer sequence, a reverse transcriptase template, where the reverse transcriptase template has less than about 85% identity to the double-stranded target polynucleotide sequence, and a primer binding sequence comprising a region complementary to a region upstream of a nick site in the double-stranded target DNA sequence, where the spacer sequence of the first member of the pair includes a sequence that is complementary to a sequence 5′ of the sequence that encodes the TINF2 DC cluster and the second member of the pair includes a spacer sequence that is complementary to a sequence 3′ of the sequence that encodes the TINF2 DC cluster; and iv) a donor sequence including a 3′ overhang that is complementary to at least a portion of the target sequence, thereby editing the TINF2 polynucleotide.

In another aspect, the present disclosure provides a method of editing an ATP1A1 polynucleotide in a cell. The method involves contacting the cell with a genome editing system including: i) a nucleic acid programmable DNA binding protein having DNA nickase activity; ii) a reverse transcriptase; iii) a pair of prime editing guide RNA (pegRNA), each including a spacer sequence, a reverse transcriptase template, where the reverse transcriptase template has less than about 85% identity to the double-stranded target polynucleotide sequence, and a primer binding sequence comprising a region complementary to a region upstream of a nick site in the double-stranded target DNA sequence, where the spacer sequence of the first member of the pair includes a sequence that is complementary to a sequence 5′ of a target nucleotide sequence in an ATP1A1 locus and the second member of the pair includes a spacer sequence that is complementary to a sequence 3′ of a target sequence in an ATP1A1 locus; and iv) a donor sequence including a 3′ overhang that hybridizes to a region of the reverse transcriptase template, thereby editing the ATP1A1 polynucleotide.

In another aspect, the present disclosure provides a method of inserting a donor sequence at a TRAC locus in a cell, the method involving contacting the cell with a genome editing system including: i) a nucleic acid programmable DNA binding protein having DNA nickase activity; ii) a reverse transcriptase; iii) a pair of prime editing guide RNA (pegRNA), each including a spacer sequence, a reverse transcriptase template, where the reverse transcriptase template has less than about 85% identity to the double-stranded target polynucleotide sequence, and a primer binding sequence including a region complementary to a region upstream of a nick site in the double-stranded target DNA sequence, where the spacer sequence of the first member of the pair includes a sequence that is complementary to a sequence 5′ of a target nucleotide sequence in a TRAC locus and the second member of the pair includes a spacer sequence that is complementary to a sequence 3′ of a target sequence in a TRAC locus; and iv) a donor sequence including a 3′ overhang that hybridizes to a region of the reverse transcriptase template, thereby inserting the donor sequence at the TRAC locus in the cell.

In another aspect, the present disclosure provides a method of editing an IL2RG polynucleotide in a cell, the method involving contacting the cell with a genome editing system including: i) a nucleic acid programmable DNA binding protein having DNA nickase activity; ii) a reverse transcriptase; iii) a pair of prime editing guide RNA (pegRNA), each including a spacer sequence, a reverse transcriptase template, where the reverse transcriptase template has less than about 85% identity to the double-stranded target polynucleotide sequence, and a primer binding sequence including a region complementary to a region upstream of a nick site in the double-stranded target DNA sequence, where the spacer sequence of the first member of the pair includes a sequence that is complementary to a sequence 5′ of a target nucleotide sequence in an IL2RG locus and the second member of the pair includes a spacer sequence that is complementary to a sequence 3′ of a target sequence in an IL2RG locus; and iv) a donor sequence including a 3′ overhang that hybridizes to a region of the reverse transcriptase template, thereby editing the IL2RG polynucleotide.

In another aspect, the present disclosure provides a method of inserting a donor sequence at an AAVS1 locus in a cell, the method involving contacting the cell with a genome editing system including: i) a nucleic acid programmable DNA binding protein having DNA nickase activity; ii) a reverse transcriptase; iii) a pair of prime editing guide RNA (pegRNA), each including a spacer sequence, a reverse transcriptase template, where the reverse transcriptase template has less than about 85% identity to the double-stranded target polynucleotide sequence, and a primer binding sequence including a region complementary to a region upstream of a nick site in the double-stranded target DNA sequence, where the spacer sequence of the first member of the pair includes a sequence that is complementary to a sequence 5′ of a target nucleotide sequence in an AAVS1 locus and the second member of the pair includes a spacer sequence that is complementary to a sequence 3′ of a target sequence in an AAVS1 locus; and iv) a donor sequence including a 3′ overhang that hybridizes to a region of the reverse transcriptase template, thereby inserting the donor sequence at the AAVS1 locus in the cell.

In another aspect, the present disclosure provides a method of treating DC in a subject. The method involves administering a paired prime editing system to the subject, where the paired prime editing system includes a prime editor including a napDNAbp, a reverse transcriptase, and a pair of pegRNAs, each including a spacer sequence complementary to a TINF2 polynucleotide including a mutation associated with DC, a primer binding sequence (PBS), and a reverse transcriptase template that encodes a wild-type TINF2 polynucleotide, or a fragment thereof.

In yet another aspect, the disclosure provides a method for editing a target genome, the method comprising contacting the target genome with the genome editing system of any previous aspect, thereby editing the target genome. In one embodiment, the genome is present in a cell in vitro or in vivo. In another embodiment, the genome is present in an organism. In another embodiment, the vector or mRNA is introduced by electroporation.

In various embodiments of any of the above aspects or any other aspect of the invention delineated herein, the donor sequence comprises an open reading frame that replaces an open reading frame present in the target polynucleotide. In various embodiments of any of the above aspects or any other aspect of the invention delineated herein, the donor sequence comprises an open reading frame that replaces an open reading frame present in the target polynucleotide. In various embodiments of any of the above aspects or any other aspect of the invention delineated herein, the donor sequence comprises an exon coding sequence, a regulatory element, or encodes a heterologous polypeptide. In various embodiments of any of the above aspects or any other aspect of the invention delineated herein, the regulatory element comprises an untranslated region, a promoter or an enhancer. In various embodiments of any of the above aspects or any other aspect of the invention delineated herein, wherein the heterologous polypeptide is a barcode, detectable moiety, or chimeric antigen receptor. In various embodiments of any of the above aspects or any other aspect of the invention delineated herein, the donor sequence corrects a mutation present in the target gene. In various embodiments of any of the above aspects or any other aspect of the invention delineated herein, the 3′ overhang overlap length comprises about 10-50 nucleotides. In various embodiments of any of the above aspects or any other aspect of the invention delineated herein, the overhang overlap length ranges from about 15 nucleotides to about 10 kb. In various embodiments of any of the above aspects or any other aspect of the invention delineated herein, the donor sequence is single stranded or double stranded. In various embodiments of any of the above aspects or any other aspect of the invention delineated herein, the donor sequence comprises an open reading frame. In various embodiments of any of the above aspects or any other aspect of the invention delineated herein, the donor sequence comprises an open reading frame that replaces an open reading frame present in the target polynucleotide. In various embodiments of any of the above aspects or any other aspect of the invention delineated herein, the donor sequence comprises an exon coding sequence, a regulatory element, or encodes a heterologous polypeptide. In various embodiments of any of the above aspects or any other aspect of the invention delineated herein, the regulatory element comprises an untranslated region, a promoter or an enhancer. In various embodiments of any of the above aspects or any other aspect of the invention delineated herein, the heterologous polypeptide is a barcode, detectable moiety, or chimeric antigen receptor. In various embodiments of any of the above aspects or any other aspect of the invention delineated herein, wherein the donor sequence corrects a mutation present in the target gene.

In any of the above aspects, or any other aspect of the invention delineated herein, the nucleic acid programmable DNA binding protein includes a CAS domain. In any of the above aspects, or embodiments thereof, the CAS domain is a CAS9, CAS12a, or CPF1 domain. In any of the above aspects, or embodiments thereof, the nucleic acid programmable DNA binding protein is fused to the reverse transcriptase. In any of the above aspects, or embodiments thereof, the 3′ end of the first pegRNA binds the target polynucleotide sequence at a site that is at least about 35 base pairs from the 3′ end of the site where the second pegRNA binds. In any of the above aspects, or embodiments thereof, the 3′ end of the first pegRNA binds the target polynucleotide sequence at a site that is at least about 45 base pairs from the 3′ end of the site where the second pegRNA binds. In any of the above aspects, or embodiments thereof, the 3′ end of the first pegRNA binds the target polynucleotide sequence at a site that is at least about 60 base pairs from the 3′ end of the site where the second pegRNA binds. In any of the above aspects, or embodiments thereof, the 3′ end of the first pegRNA binds the target polynucleotide sequence at a site that is at least about 70 base pairs from the 3′ end of the site where the second pegRNA binds. In any of the above aspects, or embodiments thereof, the 3′ end of the first pegRNA binds the target polynucleotide sequence at a site that is at least about 100 base pairs from the 3′ end of the site where the second pegRNA binds. In any of the above aspects, or embodiments thereof, the 3′ end of the first pegRNA binds the target polynucleotide sequence at a site that is at least about 200 base pairs from the 3′ end of the site where the second pegRNA binds. In any of the above aspects, or embodiments thereof, the reverse transcriptase template have less than about 80% nucleic acid sequence identity with the target polynucleotide sequence. In any of the above aspects, or embodiments thereof, the reverse transcriptase template have less than about 70% nucleic acid sequence identity with the target polynucleotide sequence. In any of the above aspects, or embodiments thereof, the reverse transcriptase template have less than about 65% nucleic acid sequence identity with the target polynucleotide sequence. In any of the above aspects, or embodiments thereof, the reverse transcriptase template have from 0% to 10% (e.g., 0, 1, 2, 3, 4, 5, 6, 7, 8, 9, 10%) nucleic acid sequence identity with the target polynucleotide sequence. In any of the above aspects, or embodiments thereof, the reverse transcriptase template are between from 10 to 40 nucleotides in length. In any of the above aspects, or embodiments thereof, the genome editing system and/or paired prime editing system further includes a viral particle X (VPX) and/or exogenous dNTPs. In any of the above aspects, or embodiments thereof, the pegRNA is an engineered pegRNA (epegRNA) further including a stabilizing sequence. In any of the above aspects, or embodiments thereof, the stabilizing sequence is downstream or 3′ of the primer binding sequence. In any of the above aspects, or embodiments thereof, the stabilizing sequence is a linker sequence and/or a pseudo-knot sequence. In any of the above aspects, or embodiments thereof, the 3′ overhang is between from 15 to 10,000 (e.g., 15, 25, 50, 75, 100, 200, 250, 500, 1,000, 2,000, 3,000, 4,000, 5,000, 6,000, 7,000, 8,000, 9,000, 10,000) nucleotides in length. In any of the above aspects, or embodiments thereof, each 3′ overhang is the same length. In any of the above aspects, or embodiments thereof, each 3′ overhang is a different length. In any of the above aspects, or embodiments thereof, the donor sequence is a plurality of single stranded polynucleotides or a double stranded polynucleotide. In any of the above aspects, or embodiments thereof, the single stranded polynucleotide and the double stranded polynucleotide comprise DNA, RNA, or a combination of DNA and RNA. In any of the above aspects, or embodiments thereof, the nucleic acid programmable DNA binding protein having DNA nickase activity and/or the reverse transcriptase is encoded by a vector or by an mRNA. In any of the above aspects, or embodiments thereof, the donor sequence includes phosphorothioate linkages. In any of the above aspects, or embodiments thereof, the donor sequence includes three phosphorothioate internucleotide linkages between the last four nucleotides at the 3′ end. In any of the above aspects, or embodiments thereof, the method is carried out in a cell in vitro or in vivo. In any of the above aspects, or embodiments thereof, the vector or mRNA is introduced by electroporation. In any of the above aspects, or embodiments thereof, the cell is derived from a subject having dyskeratosis congenita (DC). In any of the above aspects, or embodiments thereof, the editing corrects a mutation associated with DC. In any of the above aspects, or embodiments thereof, the cell is in vitro or in vivo. In any of the above aspects, or embodiments thereof, the wild-type TINF2 polynucleotide, or a fragment thereof, encodes a TINF2 DC cluster.

Compositions and articles defined by the disclosure were isolated or otherwise manufactured in connection with the examples provided below. Other features and advantages of the disclosure will be apparent from the detailed description, and from the claims.

### Definitions

Unless defined otherwise, all technical and scientific terms used herein have the meaning commonly understood by a person skilled in the art to which this disclosure belongs. The following references provide one of skill with a general definition of many of the terms used in this disclosure: Singleton et al., Dictionary of Microbiology and Molecular Biology (2nd ed. 1994); The Cambridge Dictionary of Science and Technology (Walker ed., 1988); The Glossary of Genetics, 5th Ed., R. Rieger et al. (eds.), Springer Verlag (1991); and Hale & Marham, The Harper Collins Dictionary of Biology (1991). As used herein, the following terms have the meanings ascribed to them below, unless specified otherwise.

By “agent” is meant an edited cell, a gene editing system, a polypeptide, polynucleotide, or small molecule.

By “ameliorate” is meant decrease, suppress, attenuate, diminish, arrest, or stabilize the development or progression of a disease. In some embodiments, the disease is characterized by a deleterious alteration in the genome of a cell relative to a reference genome that does not comprise said deleterious alteration.

By “alteration” is meant a change in the structure, expression levels or activity of a polynucleotide or polypeptide as detected by standard art known methods, such as those described herein. In some embodiments, the alteration is an insertion of one or more (e.g., 10, 25, 50, 100, 200, 250, 500, 750, 1,000, 2,000, 3,000 or more) nucleotides, or a deletion of one or more nucleotides. In some embodiments, the alteration is a change in the sequence of a polynucleotide relative to a reference sequence. In some embodiments, the alteration can be an increase or a decrease in activity, for example. As used herein, an alteration includes a 10% change in expression levels, a 25% change, a 40% change, and a 50% or greater change in expression levels.

By “analog” is meant a molecule that is not identical, but has analogous functional or structural features. For example, a polypeptide analog retains the biological activity of a corresponding naturally-occurring polypeptide, while having certain biochemical modifications that enhance the analog's function relative to a naturally occurring polypeptide. Such biochemical modifications could increase the analog's protease resistance, membrane permeability, or half-life, without altering, for example, ligand binding. An analog may include an unnatural amino acid.

By “ATPase, Na+/K+ transporting, alpha 1 polypeptide” or “ATP1A1 polypeptide” is meant a protein or fragment thereof having at least 85% amino acid sequence identity to the amino acid sequence of GenBank Accession Nos. NP_000692.2, NP_001153705.1, or NP_001153706.1, and having ATPase, Na+/K+ transporting, beta subunit binding activity. Exemplary ATP1A1 polypeptide sequences are found below:

```
>NP_000692.2 sodium/potassium-transporting ATPase subunit alpha-1
isoform a [Homo sapiens]
  (SEQ ID NO: 1)
MGKGVGRDKYEPAAVSEQGDKKGKKGKKDRDMDELKKEVSMDDHKLSLDELHRKYGTDLSRGLTSARAAE
ILARDGPNALTPPPTTPEWIKFCRQLFGGFSMLLWIGAILCFLAYSIQAATEEEPQNDNLYLGVVLSAVV
IITGCFSYYQEAKSSKIMESFKNMVPQQALVIRNGEKMSINAEEVVVGDLVEVKGGDRIPADLRIISANG
CKVDNSSLTGESEPQTRSPDFTNENPLETRNIAFFSTNCVEGTARGIVVYTGDRTVMGRIATLASGLEGG
QTPIAAEIEHFIHIITGVAVFLGVSFFILSLILEYTWLEAVIFLIGIIVANVPEGLLATVTVCLTLTAKR
MARKNCLVKNLEAVETLGSTSTICSDKTGTLTQNRMTVAHMWFDNQIHEADTTENQSGVSFDKTSATWLA
LSRIAGLCNRAVFQANQENLPILKRAVAGDASESALLKCIELCCGSVKEMRERYAKIVEIPFNSTNKYQL
SIHKNPNTSEPQHLLVMKGAPERILDRCSSILLHGKEQPLDEELKDAFQNAYLELGGLGERVLGFCHLFL
PDEQFPEGFQFDTDDVNFPIDNLCFVGLISMIDPPRAAVPDAVGKCRSAGIKVIMVTGDHPITAKAIAKG
VGIISEGNETVEDIAARLNIPVSQVNPRDAKACVVHGSDLKDMTSEQLDDILKYHTEIVFARTSPQQKLI
IVEGCQRQGAIVAVTGDGVNDSPALKKADIGVAMGIAGSDVSKQAADMILLDDNFASIVTGVEEGRLIFD
NLKKSIAYTLTSNIPEITPFLIFIIANIPLPLGTVTILCIDLGTDMVPAISLAYEQAESDIMKRQPRNPK
TDKLVNERLISMAYGQIGMIQALGGFFTYFVILAENGFLPIHLLGLRVDWDDRWINDVEDSYGQQWTYEQ
RKIVEFTCHTAFFVSIVVVQWADLVICKTRRNSVFQQGMKNKILIFGLFEETALAAFLSYCPGMGVALRM
YPLKPTWWFCAFPYSLLIFVYDEVRKLIIRRRPGGWVEKETYY
>NP_001153705.1 sodium/potassium-transporting ATPase subunit alpha-1
isoform c [Homo sapiens]
  (SEQ ID NO: 2)
MAFKVGRDKYEPAAVSEQGDKKGKKGKKDRDMDELKKEVSMDDHKLSLDELHRKYGTDLSRGLTSARAAE
ILARDGPNALTPPPTTPEWIKFCRQLFGGFSMLLWIGAILCFLAYSIQAATEEEPQNDNLYLGVVLSAVV
IITGCFSYYQEAKSSKIMESFKNMVPQQALVIRNGEKMSINAEEVVVGDLVEVKGGDRIPADLRIISANG
CKVDNSSLTGESEPQTRSPDFTNENPLETRNIAFFSTNCVEGTARGIVVYTGDRTVMGRIATLASGLEGG
QTPIAAEIEHFIHIITGVAVFLGVSFFILSLILEYTWLEAVIFLIGIIVANVPEGLLATVTVCLTLTAKR
MARKNCLVKNLEAVETLGSTSTICSDKTGTLTQNRMTVAHMWFDNQIHEADTTENQSGVSFDKTSATWLA
LSRIAGLCNRAVFQANQENLPILKRAVAGDASESALLKCIELCCGSVKEMRERYAKIVEIPFNSTNKYQL
SIHKNPNTSEPQHLLVMKGAPERILDRCSSILLHGKEQPLDEELKDAFQNAYLELGGLGERVLGFCHLFL
PDEQFPEGFQFDTDDVNFPIDNLCFVGLISMIDPPRAAVPDAVGKCRSAGIKVIMVTGDHPITAKAIAKG
VGIISEGNETVEDIAARLNIPVSQVNPRDAKACVVHGSDLKDMTSEQLDDILKYHTEIVFARTSPQQKLI
IVEGCQRQGAIVAVTGDGVNDSPALKKADIGVAMGIAGSDVSKQAADMILLDDNFASIVTGVEEGRLIFD
NLKKSIAYTLTSNIPEITPFLIFIIANIPLPLGTVTILCIDLGTDMVPAISLAYEQAESDIMKRQPRNPK
TDKLVNERLISMAYGQIGMIQALGGFFTYFVILAENGFLPIHLLGLRVDWDDRWINDVEDSYGQQWTYEQ
RKIVEFTCHTAFFVSIVVVQWADLVICKTRRNSVFQQGMKNKILIFGLFEETALAAFLSYCPGMGVALRM
YPLKPTWWFCAFPYSLLIFVYDEVRKLIIRRRPGGWVEKETYY
>NP_001153706.1 sodium/potassium-transporting ATPase subunit alpha-1
isoform d [Homo sapiens]
  (SEQ ID NO: 3)
MDELKKEVSMDDHKLSLDELHRKYGTDLSRGLTSARAAEILARDGPNALTPPPTTPEWIKFCRQLFGGFS
MLLWIGAILCFLAYSIQAATEEEPQNDNLYLGVVLSAVVIITGCFSYYQEAKSSKIMESFKNMVPQQALV
IRNGEKMSINAEEVVVGDLVEVKGGDRIPADLRIISANGCKVDNSSLTGESEPQTRSPDFTNENPLETRN
IAFFSTNCVEGTARGIVVYTGDRTVMGRIATLASGLEGGQTPIAAEIEHFIHIITGVAVFLGVSFFILSL
ILEYTWLEAVIFLIGIIVANVPEGLLATVTVCLTLTAKRMARKNCLVKNLEAVETLGSTSTICSDKTGTL
TQNRMTVAHMWFDNQIHEADTTENQSGVSFDKTSATWLALSRIAGLCNRAVFQANQENLPILKRAVAGDA
SESALLKCIELCCGSVKEMRERYAKIVEIPFNSTNKYQLSIHKNPNTSEPQHLLVMKGAPERILDRCSSI
LLHGKEQPLDEELKDAFQNAYLELGGLGERVLGFCHLFLPDEQFPEGFQFDTDDVNFPIDNLCFVGLISM
IDPPRAAVPDAVGKCRSAGIKVIMVTGDHPITAKAIAKGVGIISEGNETVEDIAARLNIPVSQVNPRDAK
ACVVHGSDLKDMTSEQLDDILKYHTEIVFARTSPQQKLIIVEGCQRQGAIVAVTGDGVNDSPALKKADIG
VAMGIAGSDVSKQAADMILLDDNFASIVTGVEEGRLIFDNLKKSIAYTLTSNIPEITPFLIFIIANIPLP
LGTVTILCIDLGTDMVPAISLAYEQAESDIMKRQPRNPKTDKLVNERLISMAYGQIGMIQALGGFFTYFV
ILAENGFLPIHLLGLRVDWDDRWINDVEDSYGQQWTYEQRKIVEFTCHTAFFVSIVVVQWADLVICKTRR
NSVFQQGMKNKILIFGLFEETALAAFLSYCPGMGVALRMYPLKPTWWFCAFPYSLLIFVYDEVRKLIIRR
RPGGWVEKETYY
```

By “ATPase, Na+/K+ transporting, alpha 1 polynucleotide” or “ATP1A1 polynucleotide” is meant a nucleotide encoding an ATP1A1 polypeptide. Exemplary ATP1A1 polynucleotides are found below:

```
>NM_000701.8 Homo sapiens ATPase Na+/K+ transporting subunit alpha 1
(ATP1A1), transcript variant 1, mRNA
  (SEQ ID NO: 4)
GGAGCTGCGGCGGGGTCTGGGGCGCAGAGCAGCGGCGGGAGGAGGCGGACACGTGGCAACAGCGGTAGCA
GCCCGGGCGGCGGCAGCAACAGCGGCGGCGGCATCGGCCCGAGCCGCCGGCCGCCCTCCCACCCTCCCGC
CCCGCGGCAGCCCTAGCTCCCTCCACTTGGCTCCCCTGGTCCCGCTCGCTCGGCCGGGAGCTGCTCTGTG
CTTTTCTCTCTGATTCTCCAGCGACAGGACCCGGCGCCGGGCACTGAGCACCGCCACCATGGGGAAGGGG
GTTGGACGTGATAAGTATGAGCCTGCAGCTGTTTCAGAACAAGGTGATAAAAAGGGCAAAAAGGGCAAAA
AAGACAGGGACATGGATGAACTGAAGAAAGAAGTTTCTATGGATGATCATAAACTTAGCCTTGATGAACT
TCATCGTAAATATGGAACAGACTTGAGCCGGGGATTAACATCTGCTCGTGCAGCTGAGATCCTGGCGCGA
GATGGTCCCAACGCCCTCACTCCCCCTCCCACTACTCCTGAATGGATCAAGTTTTGTCGGCAGCTCTTTG
GGGGGTTCTCAATGTTACTGTGGATTGGAGCGATTCTTTGTTTCTTGGCTTATAGCATCCAAGCTGCTAC
AGAAGAGGAACCTCAAAACGATAATCTGTACCTGGGTGTGGTGCTATCAGCCGTTGTAATCATAACTGGT
TGCTTCTCCTACTATCAAGAAGCTAAAAGTTCAAAGATCATGGAATCCTTCAAAAACATGGTCCCTCAGC
AAGCCCTTGTGATTCGAAATGGTGAGAAAATGAGCATAAATGCGGAGGAAGTTGTGGTTGGGGATCTGGT
GGAAGTAAAAGGAGGAGACCGAATTCCTGCTGACCTCAGAATCATATCTGCAAATGGCTGCAAGGTGGAT
AACTCCTCGCTCACTGGTGAATCAGAACCCCAGACTAGGTCTCCAGATTTCACAAATGAAAACCCCCTGG
AGACGAGGAACATTGCCTTCTTTTCAACCAATTGTGTTGAAGGCACCGCACGTGGTATTGTTGTCTACAC
TGGGGATCGCACTGTGATGGGAAGAATTGCCACACTTGCTTCTGGGCTGGAAGGAGGCCAGACCCCCATT
GCTGCAGAAATTGAACATTTTATCCACATCATCACGGGTGTGGCTGTGTTCCTGGGTGTGTCTTTCTTCA
TCCTTTCTCTCATCCTTGAGTACACCTGGCTTGAGGCTGTCATCTTCCTCATCGGTATCATCGTAGCCAA
TGTGCCGGAAGGTTTGCTGGCCACTGTCACGGTCTGTCTGACACTTACTGCCAAACGCATGGCAAGGAAA
AACTGCTTAGTGAAGAACTTAGAAGCTGTGGAGACCTTGGGGTCCACGTCCACCATCTGCTCTGATAAAA
CTGGAACTCTGACTCAGAACCGGATGACAGTGGCCCACATGTGGTTTGACAATCAAATCCATGAAGCTGA
TACGACAGAGAATCAGAGTGGTGTCTCTTTTGACAAGACTTCAGCTACCTGGCTTGCTCTGTCCAGAATT
GCAGGTCTTTGTAACAGGGCAGTGTTTCAGGCTAACCAGGAAAACCTACCTATTCTTAAGCGGGCAGTTG
CAGGAGATGCCTCTGAGTCAGCACTCTTAAAGTGCATAGAGCTGTGCTGTGGTTCCGTGAAGGAGATGAG
AGAAAGATACGCCAAAATCGTCGAGATACCCTTCAACTCCACCAACAAGTACCAGTTGTCTATTCATAAG
AACCCCAACACATCGGAGCCCCAACACCTGTTGGTGATGAAGGGCGCCCCAGAAAGGATCCTAGACCGTT
GCAGCTCTATCCTCCTCCACGGCAAGGAGCAGCCCCTGGATGAGGAGCTGAAAGACGCCTTTCAGAACGC
CTATTTGGAGCTGGGGGGCCTCGGAGAACGAGTCCTAGGTTTCTGCCACCTCTTTCTGCCAGATGAACAG
TTTCCTGAAGGGTTCCAGTTTGACACTGACGATGTGAATTTCCCTATCGATAATCTGTGCTTTGTTGGGC
TCATCTCCATGATTGACCCTCCACGGGCGGCCGTTCCTGATGCCGTGGGCAAATGTCGAAGTGCTGGAAT
TAAGGTCATCATGGTCACAGGAGACCATCCAATCACAGCTAAAGCTATTGCCAAAGGTGTGGGCATCATC
TCAGAAGGCAATGAGACCGTGGAAGACATTGCTGCCCGCCTCAACATCCCAGTCAGCCAGGTGAACCCCA
GGGATGCCAAGGCCTGCGTAGTACACGGCAGTGATCTAAAGGACATGACCTCCGAGCAGCTGGATGACAT
TTTGAAGTACCACACTGAGATAGTGTTTGCCAGGACCTCCCCTCAGCAGAAGCTCATCATTGTGGAAGGC
TGCCAAAGACAGGGTGCTATCGTGGCTGTGACTGGTGACGGTGTGAATGACTCTCCAGCTTTGAAGAAAG
CAGACATTGGGGTTGCTATGGGGATTGCTGGCTCAGATGTGTCCAAGCAAGCTGCTGACATGATTCTTCT
GGATGACAACTTTGCCTCAATTGTGACTGGAGTAGAGGAAGGTCGTCTGATCTTTGATAACTTGAAGAAA
TCCATTGCTTATACCTTAACCAGTAACATTCCCGAGATCACCCCGTTCCTGATATTTATTATTGCAAACA
TTCCACTACCACTGGGGACTGTCACCATCCTCTGCATTGACTTGGGCACTGACATGGTTCCTGCCATCTC
CCTGGCTTATGAGCAGGCTGAGAGTGACATCATGAAGAGACAGCCCAGAAATCCCAAAACAGACAAACTT
GTGAATGAGCGGCTGATCAGCATGGCCTATGGGCAGATTGGAATGATCCAGGCCCTGGGAGGCTTCTTTA
CTTACTTTGTGATTCTGGCTGAGAACGGCTTCCTCCCAATTCACCTGTTGGGCCTCCGAGTGGACTGGGA
TGACCGCTGGATCAACGATGTGGAAGACAGCTACGGGCAGCAGTGGACCTATGAGCAGAGGAAAATCGTG
GAGTTCACCTGCCACACAGCCTTCTTCGTCAGTATCGTGGTGGTGCAGTGGGCCGACTTGGTCATCTGTA
AGACCAGGAGGAATTCGGTCTTCCAGCAGGGGATGAAGAACAAGATCTTGATATTTGGCCTCTTTGAAGA
GACAGCCCTGGCTGCTTTCCTTTCCTACTGCCCTGGAATGGGTGTTGCTCTTAGGATGTATCCCCTCAAA
CCTACCTGGTGGTTCTGTGCCTTCCCCTACTCTCTTCTCATCTTCGTATATGACGAAGTCAGAAAACTCA
TCATCAGGCGACGCCCTGGCGGCTGGGTGGAGAAGGAAACCTACTATTAGCCCCCCGTCCTGCACGCCGT
GGAGCATCAGGCCACACACTCTGCATCCGACACCCACCCCCTCTTTGTGTACTTCAGTCTTGGAGTTTGG
AACTCTACCCTGGTAGGAAAGCACCGCAGCATGTGGGGAAGCAAGACGTCCTGGAATGAAGCATGTAGCT
CTATGGGGGGAGGGGGGAGGGCTGCCTGAAAACCATCCATCTGTGGAAATGACAGCGGGGAAGGTTTTTA
TGTGCCTTTTTGTTTTTGTAAAAAAGGAACACCCGGAAAGACTGAAAGAATACATTTTATATCTGGATTT
TTACAAATAAAGATGGCTATTATAATGGAA
>NM_001160233.2 Homo sapiens ATPase Na+/K+ transporting subunit alpha 1
(ATP1A1), transcript variant 3, mRNA
  (SEQ ID NO: 5)
AGCAGCGGGGGCGGCCCCGGGACTGAGCCGGCATCCCTGAGCCTGGCTCCCCTCCCTGCGACCGCCGTCA
CCTCCTTCTCCTTCCTTTTCCCTCCGCCCTCCGTGCCCTGAGGAAAGGCGCGCTCCTCCCCTTCCCCTGG
GGCGCTCCGCCGGGGCCTCCTCCCGGGCCTCCGTTCCCGCCGCGGCCCCGGTTCCGGCGGGGGCAGCCTC
CGGGTTCGGGGCTCCTTCTCCTGGGGACGCTGGGGCTTAGCTTGCTCCGCGCAGAGGCGGCCGCCCTCCC
CCAAAGAAAAAACTGGCTGCTTCTAAGTGCGAAGCCGGCTGGGCGGGCTGGTGCCAGAAAGGGTGTGTCT
TCACTGCCCTAAGATGGCCTTTAAGGTTGGACGTGATAAGTATGAGCCTGCAGCTGTTTCAGAACAAGGT
GATAAAAAGGGCAAAAAGGGCAAAAAAGACAGGGACATGGATGAACTGAAGAAAGAAGTTTCTATGGATG
ATCATAAACTTAGCCTTGATGAACTTCATCGTAAATATGGAACAGACTTGAGCCGGGGATTAACATCTGC
TCGTGCAGCTGAGATCCTGGCGCGAGATGGTCCCAACGCCCTCACTCCCCCTCCCACTACTCCTGAATGG
ATCAAGTTTTGTCGGCAGCTCTTTGGGGGGTTCTCAATGTTACTGTGGATTGGAGCGATTCTTTGTTTCT
TGGCTTATAGCATCCAAGCTGCTACAGAAGAGGAACCTCAAAACGATAATCTGTACCTGGGTGTGGTGCT
ATCAGCCGTTGTAATCATAACTGGTTGCTTCTCCTACTATCAAGAAGCTAAAAGTTCAAAGATCATGGAA
TCCTTCAAAAACATGGTCCCTCAGCAAGCCCTTGTGATTCGAAATGGTGAGAAAATGAGCATAAATGCGG
AGGAAGTTGTGGTTGGGGATCTGGTGGAAGTAAAAGGAGGAGACCGAATTCCTGCTGACCTCAGAATCAT
ATCTGCAAATGGCTGCAAGGTGGATAACTCCTCGCTCACTGGTGAATCAGAACCCCAGACTAGGTCTCCA
GATTTCACAAATGAAAACCCCCTGGAGACGAGGAACATTGCCTTCTTTTCAACCAATTGTGTTGAAGGCA
CCGCACGTGGTATTGTTGTCTACACTGGGGATCGCACTGTGATGGGAAGAATTGCCACACTTGCTTCTGG
GCTGGAAGGAGGCCAGACCCCCATTGCTGCAGAAATTGAACATTTTATCCACATCATCACGGGTGTGGCT
GTGTTCCTGGGTGTGTCTTTCTTCATCCTTTCTCTCATCCTTGAGTACACCTGGCTTGAGGCTGTCATCT
TCCTCATCGGTATCATCGTAGCCAATGTGCCGGAAGGTTTGCTGGCCACTGTCACGGTCTGTCTGACACT
TACTGCCAAACGCATGGCAAGGAAAAACTGCTTAGTGAAGAACTTAGAAGCTGTGGAGACCTTGGGGTCC
ACGTCCACCATCTGCTCTGATAAAACTGGAACTCTGACTCAGAACCGGATGACAGTGGCCCACATGTGGT
TTGACAATCAAATCCATGAAGCTGATACGACAGAGAATCAGAGTGGTGTCTCTTTTGACAAGACTTCAGC
TACCTGGCTTGCTCTGTCCAGAATTGCAGGTCTTTGTAACAGGGCAGTGTTTCAGGCTAACCAGGAAAAC
CTACCTATTCTTAAGCGGGCAGTTGCAGGAGATGCCTCTGAGTCAGCACTCTTAAAGTGCATAGAGCTGT
GCTGTGGTTCCGTGAAGGAGATGAGAGAAAGATACGCCAAAATCGTCGAGATACCCTTCAACTCCACCAA
CAAGTACCAGTTGTCTATTCATAAGAACCCCAACACATCGGAGCCCCAACACCTGTTGGTGATGAAGGGC
GCCCCAGAAAGGATCCTAGACCGTTGCAGCTCTATCCTCCTCCACGGCAAGGAGCAGCCCCTGGATGAGG
AGCTGAAAGACGCCTTTCAGAACGCCTATTTGGAGCTGGGGGGCCTCGGAGAACGAGTCCTAGGTTTCTG
CCACCTCTTTCTGCCAGATGAACAGTTTCCTGAAGGGTTCCAGTTTGACACTGACGATGTGAATTTCCCT
ATCGATAATCTGTGCTTTGTTGGGCTCATCTCCATGATTGACCCTCCACGGGCGGCCGTTCCTGATGCCG
TGGGCAAATGTCGAAGTGCTGGAATTAAGGTCATCATGGTCACAGGAGACCATCCAATCACAGCTAAAGC
TATTGCCAAAGGTGTGGGCATCATCTCAGAAGGCAATGAGACCGTGGAAGACATTGCTGCCCGCCTCAAC
ATCCCAGTCAGCCAGGTGAACCCCAGGGATGCCAAGGCCTGCGTAGTACACGGCAGTGATCTAAAGGACA
TGACCTCCGAGCAGCTGGATGACATTTTGAAGTACCACACTGAGATAGTGTTTGCCAGGACCTCCCCTCA
GCAGAAGCTCATCATTGTGGAAGGCTGCCAAAGACAGGGTGCTATCGTGGCTGTGACTGGTGACGGTGTG
AATGACTCTCCAGCTTTGAAGAAAGCAGACATTGGGGTTGCTATGGGGATTGCTGGCTCAGATGTGTCCA
AGCAAGCTGCTGACATGATTCTTCTGGATGACAACTTTGCCTCAATTGTGACTGGAGTAGAGGAAGGTCG
TCTGATCTTTGATAACTTGAAGAAATCCATTGCTTATACCTTAACCAGTAACATTCCCGAGATCACCCCG
TTCCTGATATTTATTATTGCAAACATTCCACTACCACTGGGGACTGTCACCATCCTCTGCATTGACTTGG
GCACTGACATGGTTCCTGCCATCTCCCTGGCTTATGAGCAGGCTGAGAGTGACATCATGAAGAGACAGCC
CAGAAATCCCAAAACAGACAAACTTGTGAATGAGCGGCTGATCAGCATGGCCTATGGGCAGATTGGAATG
ATCCAGGCCCTGGGAGGCTTCTTTACTTACTTTGTGATTCTGGCTGAGAACGGCTTCCTCCCAATTCACC
TGTTGGGCCTCCGAGTGGACTGGGATGACCGCTGGATCAACGATGTGGAAGACAGCTACGGGCAGCAGTG
GACCTATGAGCAGAGGAAAATCGTGGAGTTCACCTGCCACACAGCCTTCTTCGTCAGTATCGTGGTGGTG
CAGTGGGCCGACTTGGTCATCTGTAAGACCAGGAGGAATTCGGTCTTCCAGCAGGGGATGAAGAACAAGA
TCTTGATATTTGGCCTCTTTGAAGAGACAGCCCTGGCTGCTTTCCTTTCCTACTGCCCTGGAATGGGTGT
TGCTCTTAGGATGTATCCCCTCAAACCTACCTGGTGGTTCTGTGCCTTCCCCTACTCTCTTCTCATCTTC
GTATATGACGAAGTCAGAAAACTCATCATCAGGCGACGCCCTGGCGGCTGGGTGGAGAAGGAAACCTACT
ATTAGCCCCCCGTCCTGCACGCCGTGGAGCATCAGGCCACACACTCTGCATCCGACACCCACCCCCTCTT
TGTGTACTTCAGTCTTGGAGTTTGGAACTCTACCCTGGTAGGAAAGCACCGCAGCATGTGGGGAAGCAAG
ACGTCCTGGAATGAAGCATGTAGCTCTATGGGGGGAGGGGGGAGGGCTGCCTGAAAACCATCCATCTGTG
GAAATGACAGCGGGGAAGGTTTTTATGTGCCTTTTTGTTTTTGTAAAAAAGGAACACCCGGAAAGACTGA
AAGAATACATTTTATATCTGGATTTTTACAAATAAAGATGGCTATTATAATGGAA
>NM_001160234.2 Homo sapiens ATPase Na+/K+ transporting subunit alpha 1
(ATP1A1), transcript variant 4, mRNA
  (SEQ ID NO: 6)
GATATGTAATAATGTCTTTGCAAAGCAAAGAATATAAACAGTATAAAAGTACTAGCATTTAGATGTATTG
TATCATTTAATCCTTAAAAACATGAAATGAGGTTGGCACTATTCTTTATCTCCACGCTGTGGAAGAGGAA
ATTGAAATGTAGAAGTTAGTAACTTGCCTAAGGATACACTGCTGGTTGGACGTGATAAGTATGAGCCTGC
AGCTGTTTCAGAACAAGGTGATAAAAAGGGCAAAAAGGGCAAAAAAGACAGGGACATGGATGAACTGAAG
AAAGAAGTTTCTATGGATGATCATAAACTTAGCCTTGATGAACTTCATCGTAAATATGGAACAGACTTGA
GCCGGGGATTAACATCTGCTCGTGCAGCTGAGATCCTGGCGCGAGATGGTCCCAACGCCCTCACTCCCCC
TCCCACTACTCCTGAATGGATCAAGTTTTGTCGGCAGCTCTTTGGGGGGTTCTCAATGTTACTGTGGATT
GGAGCGATTCTTTGTTTCTTGGCTTATAGCATCCAAGCTGCTACAGAAGAGGAACCTCAAAACGATAATC
TGTACCTGGGTGTGGTGCTATCAGCCGTTGTAATCATAACTGGTTGCTTCTCCTACTATCAAGAAGCTAA
AAGTTCAAAGATCATGGAATCCTTCAAAAACATGGTCCCTCAGCAAGCCCTTGTGATTCGAAATGGTGAG
AAAATGAGCATAAATGCGGAGGAAGTTGTGGTTGGGGATCTGGTGGAAGTAAAAGGAGGAGACCGAATTC
CTGCTGACCTCAGAATCATATCTGCAAATGGCTGCAAGGTGGATAACTCCTCGCTCACTGGTGAATCAGA
ACCCCAGACTAGGTCTCCAGATTTCACAAATGAAAACCCCCTGGAGACGAGGAACATTGCCTTCTTTTCA
ACCAATTGTGTTGAAGGCACCGCACGTGGTATTGTTGTCTACACTGGGGATCGCACTGTGATGGGAAGAA
TTGCCACACTTGCTTCTGGGCTGGAAGGAGGCCAGACCCCCATTGCTGCAGAAATTGAACATTTTATCCA
CATCATCACGGGTGTGGCTGTGTTCCTGGGTGTGTCTTTCTTCATCCTTTCTCTCATCCTTGAGTACACC
TGGCTTGAGGCTGTCATCTTCCTCATCGGTATCATCGTAGCCAATGTGCCGGAAGGTTTGCTGGCCACTG
TCACGGTCTGTCTGACACTTACTGCCAAACGCATGGCAAGGAAAAACTGCTTAGTGAAGAACTTAGAAGC
TGTGGAGACCTTGGGGTCCACGTCCACCATCTGCTCTGATAAAACTGGAACTCTGACTCAGAACCGGATG
ACAGTGGCCCACATGTGGTTTGACAATCAAATCCATGAAGCTGATACGACAGAGAATCAGAGTGGTGTCT
CTTTTGACAAGACTTCAGCTACCTGGCTTGCTCTGTCCAGAATTGCAGGTCTTTGTAACAGGGCAGTGTT
TCAGGCTAACCAGGAAAACCTACCTATTCTTAAGCGGGCAGTTGCAGGAGATGCCTCTGAGTCAGCACTC
TTAAAGTGCATAGAGCTGTGCTGTGGTTCCGTGAAGGAGATGAGAGAAAGATACGCCAAAATCGTCGAGA
TACCCTTCAACTCCACCAACAAGTACCAGTTGTCTATTCATAAGAACCCCAACACATCGGAGCCCCAACA
CCTGTTGGTGATGAAGGGCGCCCCAGAAAGGATCCTAGACCGTTGCAGCTCTATCCTCCTCCACGGCAAG
GAGCAGCCCCTGGATGAGGAGCTGAAAGACGCCTTTCAGAACGCCTATTTGGAGCTGGGGGGCCTCGGAG
AACGAGTCCTAGGTTTCTGCCACCTCTTTCTGCCAGATGAACAGTTTCCTGAAGGGTTCCAGTTTGACAC
TGACGATGTGAATTTCCCTATCGATAATCTGTGCTTTGTTGGGCTCATCTCCATGATTGACCCTCCACGG
GCGGCCGTTCCTGATGCCGTGGGCAAATGTCGAAGTGCTGGAATTAAGGTCATCATGGTCACAGGAGACC
ATCCAATCACAGCTAAAGCTATTGCCAAAGGTGTGGGCATCATCTCAGAAGGCAATGAGACCGTGGAAGA
CATTGCTGCCCGCCTCAACATCCCAGTCAGCCAGGTGAACCCCAGGGATGCCAAGGCCTGCGTAGTACAC
GGCAGTGATCTAAAGGACATGACCTCCGAGCAGCTGGATGACATTTTGAAGTACCACACTGAGATAGTGT
TTGCCAGGACCTCCCCTCAGCAGAAGCTCATCATTGTGGAAGGCTGCCAAAGACAGGGTGCTATCGTGGC
TGTGACTGGTGACGGTGTGAATGACTCTCCAGCTTTGAAGAAAGCAGACATTGGGGTTGCTATGGGGATT
GCTGGCTCAGATGTGTCCAAGCAAGCTGCTGACATGATTCTTCTGGATGACAACTTTGCCTCAATTGTGA
CTGGAGTAGAGGAAGGTCGTCTGATCTTTGATAACTTGAAGAAATCCATTGCTTATACCTTAACCAGTAA
CATTCCCGAGATCACCCCGTTCCTGATATTTATTATTGCAAACATTCCACTACCACTGGGGACTGTCACC
ATCCTCTGCATTGACTTGGGCACTGACATGGTTCCTGCCATCTCCCTGGCTTATGAGCAGGCTGAGAGTG
ACATCATGAAGAGACAGCCCAGAAATCCCAAAACAGACAAACTTGTGAATGAGCGGCTGATCAGCATGGC
CTATGGGCAGATTGGAATGATCCAGGCCCTGGGAGGCTTCTTTACTTACTTTGTGATTCTGGCTGAGAAC
GGCTTCCTCCCAATTCACCTGTTGGGCCTCCGAGTGGACTGGGATGACCGCTGGATCAACGATGTGGAAG
ACAGCTACGGGCAGCAGTGGACCTATGAGCAGAGGAAAATCGTGGAGTTCACCTGCCACACAGCCTTCTT
CGTCAGTATCGTGGTGGTGCAGTGGGCCGACTTGGTCATCTGTAAGACCAGGAGGAATTCGGTCTTCCAG
CAGGGGATGAAGAACAAGATCTTGATATTTGGCCTCTTTGAAGAGACAGCCCTGGCTGCTTTCCTTTCCT
ACTGCCCTGGAATGGGTGTTGCTCTTAGGATGTATCCCCTCAAACCTACCTGGTGGTTCTGTGCCTTCCC
CTACTCTCTTCTCATCTTCGTATATGACGAAGTCAGAAAACTCATCATCAGGCGACGCCCTGGCGGCTGG
GTGGAGAAGGAAACCTACTATTAGCCCCCCGTCCTGCACGCCGTGGAGCATCAGGCCACACACTCTGCAT
CCGACACCCACCCCCTCTTTGTGTACTTCAGTCTTGGAGTTTGGAACTCTACCCTGGTAGGAAAGCACCG
CAGCATGTGGGGAAGCAAGACGTCCTGGAATGAAGCATGTAGCTCTATGGGGGGAGGGGGGAGGGCTGCC
TGAAAACCATCCATCTGTGGAAATGACAGCGGGGAAGGTTTTTATGTGCCTTTTTGTTTTTGTAAAAAAG
GAACACCCGGAAAGACTGAAAGAATACATTTTATATCTGGATTTTTACAAATAAAGATGGCTATTATAAT
GGAA
```

By “barcode” is meant a degenerate or semi-degenerate nucleic acid sequence that varies plasmid to plasmid or genome to genome. The barcode sequence may be a degenerate or a semi-degenerate sequence that is identifiable. For example, a barcode may comprise identifiable degenerate sequences that have several possible bases in any of the positions of the nucleic acid sequence. A barcode may uniquely label or detect a single polynucleotide or cell.

By “DC cluster” is meant amino acids 270-300 of a TINF2 polypeptide described herein below or amino acids corresponding to those in another TINF2 amino acid sequence. In some embodiments, the DC cluster comprises or consists of

(SEQ ID NO: 7)
QSQWASTRGGHKERPTVMLFPFRNLGSPTQ.

In this disclosure, “comprises,” “comprising,” “containing” and “having” and the like can have the meaning ascribed to them in U.S. patent law and can mean “includes,” “including,” and the like; “consisting essentially of” or “consists essentially” likewise has the meaning ascribed in U.S. patent law and the term is open-ended, allowing for the presence of more than that which is recited so long as basic or novel characteristics of that which is recited is not changed by the presence of more than that which is recited, but excludes prior art embodiments. Any embodiments specified as “comprising” a particular component(s) or element(s) are also contemplated as “consisting of” or “consisting essentially of” the particular component(s) or element(s) in some embodiments.

By “consist essentially” it is meant that the ingredients include only the listed components along with the normal impurities present in commercial materials and with any other additives present at levels which do not affect the operation of the disclosure, for instance at levels less than 5% by weight or less than 1% or even 0.5% by weight.

“Detect” refers to identifying the presence, absence or amount of the analyte to be detected. In some embodiments, the analyte is the presence of an edit. In some embodiments, the efficiency of editing is characterized. Means of characterizing editing include Sanger sequencing and next-generation sequencing (e.g., short-read sequencing). Means of analyzing Sanger sequencing include Inference of CRISPR Edits (ICE), Tracking of Indels by Decomposition (TIDE) analysis, and Base Editing Analysis Tool (BEAT). Means of analyzing next-generation sequencing include CRISPResso

By “detectable moiety” is meant a composition that when linked to a molecule of interest renders the latter detectable, via spectroscopic, photochemical, biochemical, immunochemical, or chemical means. For example, useful labels include radioactive isotopes, magnetic beads, metallic beads, colloidal particles, fluorescent dyes, electron-dense reagents, enzymes (for example, as commonly used in an ELISA), biotin, digoxigenin, or haptens.

By “disease” is meant any condition or disorder that damages or interferes with the normal function of a cell, tissue, or organ. Exemplary diseases include those associated with a deleterious genetic alteration. In an embodiment, the disease is dyskeratosis congenita.

By “effective amount” is meant the amount of an agent required to ameliorate the symptoms of a disease relative to an untreated patient. In an embodiment, an effective amount refers to the amount of a genome editing system described herein required to alter the genome of one or more cells of a subject. In another embodiment, an effective amount refers to the amount of modified cells required to ameliorate the effect of a deleterious genetic mutation in the subject, wherein a modified cell is a cell edited using a genome editing technology described herein. The effective amount of active compound(s) used to practice the present invention for therapeutic treatment of a disease varies depending upon the manner of administration, the age, body weight, and general health of the subject. Ultimately, the attending physician or veterinarian will decide the appropriate amount and dosage regimen. Such amount is referred to as an “effective” amount.

The invention provides a number of targets that are useful for the development of highly specific drugs or agents to treat a disorder characterized by the methods delineated herein. In addition, the methods of the invention provide a facile means to identify therapies that are safe for use in subjects. In addition, the methods of the invention provide a route for analyzing virtually any number of compounds for effects on a disease described herein with high-volume throughput, high sensitivity, and low complexity.

By “fragment” is meant a portion of a polypeptide or nucleic acid molecule. In embodiments, portion contains, at least 10%, 20%, 30%, 40%, 50%, 60%, 70%, 80%, or 90% of the entire length of the reference nucleic acid molecule or polypeptide. A fragment may contain 10, 20, 30, 40, 50, 60, 70, 80, 90, or 100, 200, 300, 400, 500, 600, 700, 800, 900, 1000, 1,500, 2,000, 2,500, 3,000, 3,500, 4,000, 4,500, 5,000, 5,500, 6,000, 6,500, 7,000, 7,500, 8,000, 8,500, 9,000, 9,500, 10,000, 11,000, 12,000, 13,000, 14,000, 15,000, 16,000, 17,000, 18,000, 19,000, 20,000, 21,000, 22,000, 23,000, 24,000, 25,000, 26,000, 27,000, 28,000, 29,000, or 30,000 nucleotides or amino acids. In some embodiments, a fragment may contain at least about 1 kb, 3 kb, 5 kb, 10 kb, 15 kb, 20 kb, or 30 kb. In embodiments, polynucleotide fragments (also termed donor sequences) are inserted into the genome of a cell.

“Hybridization” means hydrogen bonding, which may be Watson-Crick, Hoogsteen or reversed Hoogsteen hydrogen bonding, between complementary nucleobases. For example, adenine and thymine are complementary nucleobases that pair through the formation of hydrogen bonds.

By “gene editing system” is meant the polypeptides, polynucleotides, and other reagents involved in introducing an alteration to a polynucleotide sequence. In embodiments, the gene editing system is a modified paired prime editing system.

By “guide polynucleotide” is meant a polynucleotide or polynucleotide complex which is specific for a target sequence and can form a complex with a polynucleotide programmable nucleotide binding domain protein (e.g., Cas9 or Cpf1). In an embodiment, the guide polynucleotide is a guide RNA (gRNA), such as an prime editing guide RNA (pegRNA). Advantageously, paired prime editing systems described herein employ a pair of prime editing guide RNAs (pegRNAs) that comprise a reverse transcriptase template, such that that the reverse transcriptase template has less than about 85% nucleotide sequence identity with a target sequence. In some embodiments, the reverse transcriptase template has less than about 90%, less than about 80%, less than about 75%, less than about 70%, less than about 65%, less than about 60%, less than about 55%, less than about 50%, less than about 45%, less than about 40%, less than about 35%, less than about 30%, less than about 25%, less than about 20%, less than about 15%, less than about 10%, less than about 5%, or about 0% nucleotide sequence identity with a target sequence. In some embodiments, the reverse transcriptase template is codon-optimized, while in other embodiments, the reverse transcriptase template is not codon-optimized. In another embodiment, a gRNA can exist as a complex of two or more RNAs, or as a single RNA molecule. In some embodiments, the pegRNA may be an engineered prime editing guide RNA (epegRNA), having, for example, improved stability.

By “heterologous,” or “exogenous” is meant a polynucleotide or polypeptide that 1) has been experimentally incorporated to a polynucleotide or polypeptide sequence to which the polynucleotide or polypeptide is not normally found in nature; or 2) has been experimentally placed into a cell that does not normally comprise the polynucleotide or polypeptide.

By “Interleukin 2 receptor subunit gamma polypeptide” or “IL2RG polypeptide” is meant a protein or fragment thereof having at least 85% amino acid sequence identity to the amino acid sequence of GenBank Accession No. NP_000197.1, and having Interleukin 2 subunit binding activity. An exemplary IL2RG polypeptide sequence is found below:

>NP_000197.1 cytokine receptor common subunit gamma precursor [Homo sapiens]
(SEQ ID NO: 8)
MLKPSLPFTSLLFLQLPLLGVGLNTTILTPNGNEDTTADFFLTTMPTDSLSVSTLPLPEVQCFVENVEYM
NCTWNSSSEPQPTNLTLHYWYKNSDNDKVQKCSHYLFSEEITSGCQLQKKEIHLYQTFVVQLQDPREPRR
QATQMLKLQNLVIPWAPENLTLHKLSESQLELNWNNRFLNHCLEHLVQYRTDWDHSWTEQSVDYRHKFSL
PSVDGQKRYTFRVRSRFNPLCGSAQHWSEWSHPIHWGSNTSKENPFLFALEAVVISVGSMGLIISLLCVY
FWLERTMPRIPTLKNLEDLVTEYHGNFSAWSGVSKGLAESLQPDYSERLCLVSEIPPKGGALGEGPGASP
CNQHSPYWAPPCYTLKPET

By “Interleukin 2 receptor subunit gamma polynucleotide” or “IL2RG polynucleotide” is meant a nucleotide encoding an IL2RG polypeptide. Exemplary IL2RG polynucleotides are found below:

>NM_000206.3 Homo sapiens interleukin 2 receptor subunit gamma (IL2RG), mRNA
(SEQ ID NO: 9)
ACAGACAGACTACACCCAGGGAATGAAGAGCAAGCGCCATGTTGAAGCCATCATTACCATTCACATCCCT
CTTATTCCTGCAGCTGCCCCTGCTGGGAGTGGGGCTGAACACGACAATTCTGACGCCCAATGGGAATGAA
GACACCACAGCTGATTTCTTCCTGACCACTATGCCCACTGACTCCCTCAGTGTTTCCACTCTGCCCCTCC
CAGAGGTTCAGTGTTTTGTGTTCAATGTCGAGTACATGAATTGCACTTGGAACAGCAGCTCTGAGCCCCA
GCCTACCAACCTCACTCTGCATTATTGGTACAAGAACTCGGATAATGATAAAGTCCAGAAGTGCAGCCAC
TATCTATTCTCTGAAGAAATCACTTCTGGCTGTCAGTTGCAAAAAAAGGAGATCCACCTCTACCAAACAT
TTGTTGTTCAGCTCCAGGACCCACGGGAACCCAGGAGACAGGCCACACAGATGCTAAAACTGCAGAATCT
GGTGATCCCCTGGGCTCCAGAGAACCTAACACTTCACAAACTGAGTGAATCCCAGCTAGAACTGAACTGG
AACAACAGATTCTTGAACCACTGTTTGGAGCACTTGGTGCAGTACCGGACTGACTGGGACCACAGCTGGA
CTGAACAATCAGTGGATTATAGACATAAGTTCTCCTTGCCTAGTGTGGATGGGCAGAAACGCTACACGTT
TCGTGTTCGGAGCCGCTTTAACCCACTCTGTGGAAGTGCTCAGCATTGGAGTGAATGGAGCCACCCAATC
CACTGGGGGAGCAATACTTCAAAAGAGAATCCTTTCCTGTTTGCATTGGAAGCCGTGGTTATCTCTGTTG
GCTCCATGGGATTGATTATCAGCCTTCTCTGTGTGTATTTCTGGCTGGAACGGACGATGCCCCGAATTCC
CACCCTGAAGAACCTAGAGGATCTTGTTACTGAATACCACGGGAACTTTTCGGCCTGGAGTGGTGTGTCT
AAGGGACTGGCTGAGAGTCTGCAGCCAGACTACAGTGAACGACTCTGCCTCGTCAGTGAGATTCCCCCAA
AAGGAGGGGCCCTTGGGGAGGGGCCTGGGGCCTCCCCATGCAACCAGCATAGCCCCTACTGGGCCCCCCC
ATGTTACACCCTAAAGCCTGAAACCTGAACCCCAATCCTCTGACAGAAGAACCCCAGGGTCCTGTAGCCC
TAAGTGGTACTAACTTTCCTTCATTCAACCCACCTGCGTCTCATACTCACCTCACCCCACTGTGGCTGAT
TTGGAATTTTGTGCCCCCATGTAAGCACCCCTTCATTTGGCATTCCCCACTTGAGAATTACCCTTTTGCC
CCGAACATGTTTTTCTTCTCCCTCAGTCTGGCCCTTCCTTTTCGCAGGATTCTTCCTCCCTCCCTCTTTC
CCTCCCTTCCTCTTTCCATCTACCCTCCGATTGTTCCTGAACCGATGAGAAATAAAGTTTCTGTTGATAA
TCATCAAAAA

By “increase” is meant to alter positively relative to a reference. An increase may be by 1%, 5%, 10%, 25%, 30%, 50%, 75%, 100%, or more, or by 1.5-fold, -fold 2-fold, 3-fold, 4-fold, 5-fold, 6-fold, 7-fold, 8-fold, 9-fold, 10-fold, 25-fold, 50-fold, 75-fold, 100-fold, or more.

The terms “isolated,” “purified,” or “biologically pure” refer to material that is free to varying degrees from components which normally accompany it as found in its native state. “Isolate” denotes a degree of separation from original source or surroundings. “Purify” denotes a degree of separation that is higher than isolation. A “purified” or “biologically pure” protein is sufficiently free of other materials such that any impurities do not materially affect the biological properties of the protein or cause other adverse consequences. That is, a nucleic acid or peptide of this invention is purified if it is substantially free of cellular material, viral material, or culture medium when produced by recombinant DNA techniques, or chemical precursors or other chemicals when chemically synthesized. Purity and homogeneity are typically determined using analytical chemistry techniques, for example, polyacrylamide gel electrophoresis or high performance liquid chromatography. The term “purified” can denote that a nucleic acid or protein gives rise to essentially one band in an electrophoretic gel. For a protein that can be subjected to modifications, for example, phosphorylation or glycosylation, different modifications may give rise to different isolated proteins, which can be separately purified.

By “isolated polynucleotide” is meant a nucleic acid that is free of the genes which, in the naturally-occurring genome of the organism from which the nucleic acid molecule of the invention is derived, flank the gene. The term therefore includes, for example, a recombinant DNA that is incorporated into a vector; into an autonomously replicating plasmid or virus; or into the genomic DNA of a prokaryote or eukaryote; or that exists as a separate molecule (for example, a cDNA or a genomic or cDNA fragment produced by PCR or restriction endonuclease digestion) independent of other sequences. In addition, the term includes an RNA molecule that is transcribed from a DNA molecule, as well as a recombinant DNA that is part of a hybrid gene encoding additional polypeptide sequence.

By an “isolated polypeptide” is meant a polypeptide of the invention that has been separated from components that naturally accompany it. Typically, the polypeptide is isolated when it is at least 60%, by weight, free from the proteins and naturally-occurring organic molecules with which it is naturally associated. In embodiments, the preparation is at least 75%, at least 90%, and or at least 99%, by weight, a polypeptide of the invention. An isolated polypeptide of the invention may be obtained, for example, by extraction from a natural source, by expression of a recombinant nucleic acid encoding such a polypeptide; or by chemically synthesizing the protein. Purity can be measured by any appropriate method, for example, column chromatography, polyacrylamide gel electrophoresis, or by HPLC analysis.

As used herein, “obtaining” as in “obtaining an agent” includes synthesizing, purchasing, or otherwise acquiring the agent.

As used herein, the terms “prevent,” “preventing,” “prevention,” “prophylactic treatment” and the like refer to reducing the probability of developing a disorder or condition in a subject, who does not have, but is at risk of or susceptible to developing a disorder or condition.

By “polypeptide” or “amino acid sequence” is meant any chain of amino acids, regardless of length or post-translational modification. In various embodiments, the post-translational modification is glycosylation or phosphorylation. In various embodiments, conservative amino acid substitutions may be made to a polypeptide to provide functionally equivalent variants, or homologs of the polypeptide. In some aspects the invention embraces sequence alterations that result in conservative amino acid substitutions. In some embodiments, a “conservative amino acid substitution” refers to an amino acid substitution that does not alter the relative charge or size characteristics of the protein in which the conservative amino acid substitution is made. Variants can be prepared according to methods for altering polypeptide sequence known to one of ordinary skill in the art such as are found in references that compile such methods, e.g. Molecular Cloning: A Laboratory Manual, J. Sambrook, et al., eds., Second Edition, Cold Spring Harbor Laboratory Press, Cold Spring Harbor, N.Y., 1989, or Current Protocols in Molecular Biology, F. M. Ausubel, et al., eds., John Wiley & Sons, Inc., New York. Non-limiting examples of conservative substitutions of amino acids include substitutions made among amino acids within the following groups: (a) M, I, L, V; (b) F, Y, W; (c) K, R, H; (d) A, G; (e) S, T; (f) Q, N; and (g) E, D. In various embodiments, conservative amino acid substitutions can be made to the amino acid sequence of the proteins and polypeptides disclosed herein.

By “prime editing” is meant gene editing that involves the use of a nucleic acid programmable DNA binding protein (napDNAbp) having nickase activity, a reverse transcriptase, a guide RNA that guides the napDNAbp to a target sequence to generate a single stranded nick at the target site, and uses the nicked DNA as a primer for reverse transcription of an engineered reverse transcriptase template that is integrated with the guide RNA. In one embodiment, the napDNAbp is a fusion protein comprising a Cas nickase and a reverse transcriptase. Specifics of prime editing and prime editing systems are known in the art and described, for example, in U.S. Pat. No. 11,447,770, which is incorporated herein in its entirety. Modified paired prime editing (“Prime Assembly”), as described herein, is a form of prime editing where two pegRNAs comprising reverse transcriptase templates are used to accomplish genome editing by inserting single or double stranded donor sequences into a target polynucleotide. This approach facilitates the insertion of long (e.g., greater than 100 nucleotide) donor sequences (e.g., single or double stranded DNA) into a target site on a target polynucleotide, while advantageously removing any requirement for using long reverse transcriptase template sequences (e.g., >20-25 nt) to effect this insertion.

By “paired prime editing system” or “paired prime editing” is meant
i) a nucleic acid programmable DNA binding protein having DNA nickase activity fused to or that associates with a reverse transcriptase domain;
ii) two prime editing guide RNAs, each comprising a spacer sequence complementary to the first strand of a double-stranded target polynucleotide sequence, a reverse transcriptase template (also termed a DNA synthesis template), and a primer binding sequence comprising a region complementary to a region upstream of a nick site in the second strand of the double-stranded target DNA sequence. In an embodiment, the paired prime editing system comprises a fusion protein comprising a programmable DNA binding protein (e.g., CAS9) domain having nickase activity fused to a reverse transcriptase, and two prime editing guide RNAs (pegRNA). In an embodiment, a prime editing target polynucleotide comprises a double stranded DNA molecule having two complementary strands: a first strand that may be referred to as a “target strand” or a “non-edit strand”, and a second strand that may be referred to as a “non-target strand,” or an “edit strand.” Exemplary prime editing systems are described, for example, in U.S. Pat. No. 11,447,770, which is incorporated herein by reference in its entirety. Paired pegRNA prime editing systems include Twin Prime Editing (TwinPE), Prime Editing-Cas9-based deletion and repair (PEDAR), genome editing by RTTs partially aligned to each other but nonhomologous to target sequences within duo pegRNA (GRAND), PrimeDEL, and bi-direction Prime Editing (Bi-PE).

By “programmable DNA binding protein” is meant a polypeptide capable of binding DNA, where the specificity of binding is provided by its interaction with a polynucleotide or polypeptide that guides the protein to its binding target. In some embodiments, the programmable DNA binding protein interacts with a polynucleotide, such as a guide RNA (e.g., prime editing guide RNA). In some embodiments, the programmable DNA binding protein is a Cas9, Cas12 (e.g., Cas12a, 12b), or Cas13.

By “reduce” is meant to alter negatively relative to a reference. A reduction may be by 1%, 5%, 10%, 25%, 30%, 50%, 75%, 100%, or more, or by 1.5-fold, -fold 2-fold, 3-fold, 4-fold, 5-fold, 6-fold, 7-fold, 8-fold, 9-fold, 10-fold, 25-fold, 50-fold, 75-fold, 100-fold, or more.

By “reference” is meant a standard or control condition. In some embodiments, a modified cell comprising a genome edited using the genome editing technology described herein is compared to an unmodified cell (i.e., a cell having an unedited genome). In some embodiments, a cell edited to repair or replace a mutation associated with dyskeratosis congenita is compared to a cell comprising a mutation associated with dyskeratosis congenita. In some cases, the reference is a healthy cell or a healthy subject, or the reference is a cell or subject that does not have or is not associated with a disease (e.g., dyskeratosis congenita).

A “reference sequence” is a defined sequence used as a basis for sequence comparison. A reference sequence may be a subset of or the entirety of a specified sequence; for example, a segment of a full-length cDNA or gene sequence, or the complete cDNA or gene sequence. For polypeptides, the length of the reference polypeptide sequence will generally be at least about 16 amino acids, at least about 20 amino acids, at least about 25 amino acids, at least about 35 amino acids, at least about 50 amino acids, or at least about 100 amino acids. For nucleic acids, the length of the reference nucleic acid sequence will generally be at least about 50 nucleotides, at least about 60 nucleotides, at least about 75 nucleotides, at least about 100 nucleotides, or at least about 300 nucleotides, or any integer thereabout or therebetween. In some embodiments, the reference sequence is the sequence of a reference genome. In some embodiments, a reference sequence is the sequence of a polynucleotide, gene, or genome prior to editing with a gene editing system described herein.

By “region” is meant a portion of a polynucleotide or polypeptide. A region may comprise between about 5, 10, 15, 20, 25, 30, 35, 40, 45, 50, 60, 70, 80, 90, 100, 200, 300, 400, or 500 nucleotides or amino acids. In other embodiments, a region of a polypeptide comprises a domain or structural feature, and a region of a polynucleotide comprises the polynucleotides encoding that domain or that feature.

By “specifically binds” is meant a polypeptide or polynucleotide that recognizes and binds a target polypeptide or polynucleotide, but which does not substantially recognize and bind other molecules in a sample, for example, a biological sample.

Nucleic acid molecules useful in the methods of the invention include any nucleic acid molecule that encodes a polypeptide of the invention or a fragment thereof. Such nucleic acid molecules need not be 100% identical with an endogenous nucleic acid sequence, but will typically exhibit substantial identity. Polynucleotides having “substantial identity” to an endogenous sequence are typically capable of hybridizing with at least one strand of a double-stranded nucleic acid molecule. Nucleic acid molecules useful in the methods of the invention include any nucleic acid molecule that encodes a polypeptide of the invention or a fragment thereof. Such nucleic acid molecules need not be 100% identical with an endogenous nucleic acid sequence, but will typically exhibit substantial identity. Polynucleotides having “substantial identity” to an endogenous sequence are typically capable of hybridizing with at least one strand of a double-stranded nucleic acid molecule. By “hybridize” is meant pair to form a double-stranded molecule between complementary polynucleotide sequences (e.g., a gene described herein), or portions thereof, under various conditions of stringency. (See, e.g., Wahl, G. M. and S. L. Berger (1987) Methods Enzymol. 152:399; Kimmel, A. R. (1987) Methods Enzymol. 152:507).

For example, stringent salt concentration will ordinarily be less than about 750 mM NaCl and 75 mM trisodium citrate, about less than about 500 mM NaCl and 50 mM trisodium citrate, or about less than about 250 mM NaCl and 25 mM trisodium citrate. Low stringency hybridization can be obtained in the absence of organic solvent, e.g., formamide, while high stringency hybridization can be obtained in the presence of at least about 35% formamide, or at least about 50% formamide. Stringent temperature conditions will ordinarily include temperatures of at least about 30° C., of at least about 37° C., or of at least about 42° C. Varying additional parameters, such as hybridization time, the concentration of detergent, e.g., sodium dodecyl sulfate (SDS), and the inclusion or exclusion of carrier DNA, are well known to those skilled in the art. Various levels of stringency are accomplished by combining these various conditions as needed. In an embodiment, hybridization will occur at 30° C. in 750 mM NaCl, 75 mM trisodium citrate, and 1% SDS. In another embodiment, hybridization will occur at 37° C. in 500 mM NaCl, 50 mM trisodium citrate, 1% SDS, 35% formamide, and 100 μg/ml denatured salmon sperm DNA (ssDNA). In another embodiment, hybridization will occur at 42° C. in 250 mM NaCl, 25 mM trisodium citrate, 1% SDS, 50% formamide, and 200 μg/ml ssDNA. Useful variations on these conditions will be readily apparent to those skilled in the art.

For most applications, washing steps that follow hybridization will also vary in stringency. Wash stringency conditions can be defined by salt concentration and by temperature. As above, wash stringency can be increased by decreasing salt concentration or by increasing temperature. For example, stringent salt concentration for the wash steps will be less than about 30 mM NaCl and 3 mM trisodium citrate, or less than about 15 mM NaCl and 1.5 mM trisodium citrate. Stringent temperature conditions for the wash steps will ordinarily include a temperature of at least about 25° C., of at least about 42° C., or of at least about 68° C. In another embodiment, wash steps will occur at 25° C. in 30 mM NaCl, 3 mM trisodium citrate, and 0.1% SDS. In another embodiment, wash steps will occur at 42° C. in 15 mM NaCl, 1.5 mM trisodium citrate, and 0.1% SDS. In another embodiment, wash steps will occur at 68° C. in 15 mM NaCl, 1.5 mM trisodium citrate, and 0.1% SDS. Additional variations on these conditions will be readily apparent to those skilled in the art. Hybridization techniques are well known to those skilled in the art and are described, for example, in Benton and Davis (Science 196:180, 1977); Grunstein and Hogness (Proc. Natl. Acad. Sci., USA 72:3961, 1975); Ausubel et al. (Current Protocols in Molecular Biology, Wiley Interscience, New York, 2001); Berger and Kimmel (Guide to Molecular Cloning Techniques, 1987, Academic Press, New York); and Sambrook et al., Molecular Cloning: A Laboratory Manual, Cold Spring Harbor Laboratory Press, New York.

By “substantially identical” is meant a polypeptide or nucleic acid molecule exhibiting at least 50% identity to a reference amino acid sequence (for example, any one of the amino acid sequences described herein) or nucleic acid sequence (for example, any one of the nucleic acid sequences described herein). In embodiments, such a sequence is at least 60%, at least 80% or 85%, or at least about 90%, 95% or even 99% identical at the amino acid level or nucleic acid to the sequence used for comparison.

Sequence identity is typically measured using sequence analysis software (for example, Sequence Analysis Software Package of the Genetics Computer Group, University of Wisconsin Biotechnology Center, 1710 University Avenue, Madison, Wis. 53705, BLAST, BESTFIT, GAP, or PILEUP/PRETTYBOX programs). Such software matches identical or similar sequences by assigning degrees of homology to various substitutions, deletions, and/or other modifications. Conservative substitutions typically include substitutions within the following groups: glycine, alanine; valine, isoleucine, leucine; aspartic acid, glutamic acid, asparagine, glutamine; serine, threonine; lysine, arginine; and phenylalanine, tyrosine. In an exemplary approach to determining the degree of identity, a BLAST program may be used, with a probability score between e⁻³ and e⁻¹⁰⁰ indicating a closely related sequence.

By “subject” is meant an animal. The animal can be a mammal. The mammal can be a human or non-human mammal, such as a bovine, equine, canine, ovine, rodent, or feline. In an embodiment, the subject has dyskeratosis congenita.

Ranges provided herein are understood to be shorthand for all of the values within the range. For example, a range of 1 to 50 is understood to include any number, combination of numbers, or sub-range from the group consisting of 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50.

The term “target site” refers to a nucleotide sequence or nucleobase of interest that is modified. In embodiments, the target site exists within a larger polynucleotide molecule (e.g., DNA, gene, genome). In an embodiment, the target site is present in a target polynucleotide.

As used herein, the terms “treat,” “treating,” “treatment,” and the like refer to reducing or ameliorating a disorder and/or symptoms associated therewith. It will be appreciated that, although not precluded, treating a disorder or condition does not require that the disorder, condition or symptoms associated therewith be completely eliminated.

By “TERF1 interacting nuclear factor 2 polypeptide” or “TINF2 polypeptide” is meant a protein or fragment thereof having at least 85% amino acid sequence identity to the amino acid sequence of GenBank Accession Nos. AAH19343.1 or CCDS41936.1 and having telomeric repeat factor 1 (TRF1) binding activity. The sequences of exemplary TINF2 polypeptides may be found below, with the DC cluster bolded, and amino acids most commonly found to be mutated in dyskeratosis congenita in bold underline:

```
>AAH19343. 1 TINF2 protein [Homo sapiens]
  (SEQ ID NO: 10)
MATPLVAGPAALRFAAAASWQVVRGRCVEHFPRVLEFLRSLRAVAPGLVRYRHHERLCMGLKAK
VVVELILQGRPWAQVLKALNHHFPESGPIVRDPKATKQDLRKILEAQETFYQQVKQLSEAPVDL
ASKLQELEQEYGEPFLAAMEKLLFEYLCQLEKALPTPQAQQLQDVLSWMQPGVSITSSLAWRQY
GVDMGWLLPECSVTDSVNLAEPMEQNPPQQQRLALHNPLPKAKPGTHLPQGPSSRTHPEPLAGR
HFNLAPLGRRRVQSQWASTRGGHKERPTVMLFPFRNLGSPTQVISKPESKEEHAIYTADLAMGT
RAASTGKSKSPCQTLGGRALKENPVDLPATEQKENCLDCYMDPLRLSLLPPRARKPVCPPSLCS
SVITIGDLVLDSDEEENGQGEGKESLENYQKTKFDTLIPTLCEYLPPSGHGAIPVSSCDCRDSS
RPL
CCDS41937.1
  (SEQ ID NO: 11)
MATPLVAGPAALRFAAAASWQVVRGRCVEHFPRVLEFLRSLRAVAPGLVRYRHHERLCMGLKAK
VVVELILQGRPWAQVLKALNHHFPESGPIVRDPKATKQDLRKILEAQETFYQQVKQLSEAPVDL
ASKLQELEQEYGEPFLAAMEKLLFEYLCQLEKALPTPQAQQLQDVLSWMQPGVSITSSLAWRQY
GVDMGWLLPECSVTDSVNLAEPMEQNPPQQQRLALHNPLPKAKPGTHLPQGPSSRTHPEPLAGR
HFNLAPLGRRRVQSQWASTRGGHKERPTVMLFPFRNLGSPTQVISKPESKEEHAIYTADLAMGT
RAASTGKSKSPCQTLGGRALKENPVDLPATEQKE
```

Commonly mutated amino acid residues in the TINF2 polypeptide found in Dyskeratosis Congenita (DC) include: K280E; K280X; R282H; R282S; R282C; P283S; P283A; P283H; T284A; T284fs (frameshift); L287P; P289S; F290fs (frameshift); and R291G.

The TINF2 polypeptide comprises a “DC cluster” that comprises amino acids 270-300 of the above-referenced TINF2 polypeptide or amino acids corresponding to those in another TINF2 amino acid sequence. In some embodiments, the DC cluster comprises or consists of QSQWASTRGGHKERETVMLFPFRNLGSPTQ (SEQ ID NO: 7).

By “TERF1 interacting nuclear factor 2 polynucleotide” or “TINF2 polynucleotide” is meant a nucleic acid molecule encoding a TINF2 polypeptide or fragment thereof. In embodiments, a TINF2 polynucleotide sequence is provided at NM_001099274.3, NM 001363668.2, or at NM 012461.3.

Exemplary TINF2 polynucleotide sequences are provided below with the DC cluster bolded, and nucleotides most commonly found to be mutated in dyskeratosis congenita in bold underline:

```
>NM_001099274.3 Homo sapiens TERF1 interacting nuclear factor 2
(TINF2), transcript variant 1, mRNA
  (SEQ ID NO: 12)
GAGGCACCCTCGGGCTCGAGACAGCGGCGACGTTTAAAGCTGAGCGACCCAGTGCCACTGGAGACGGTCA
GCTTCTCCACTCAGGCTCCTCCAGCCCGAGCCAGAAGACCCCCTCCCCCAGAATTCTGGGGGCCGATGGA
AGGGAGCCGAGTCAGATCGCGAGGTACCCAGAGCCGACAGACCGGAGCGACAGGGAGTTGCCAGAAGCCC
CGCCCCTAGGAGTGATCGGAAAGCCTCACCCATCCGGGTGAGGAACCCGGAGGGACCGCCTCCGGGCGGA
GCCCGCCGACCATGGCTACGCCCCTGGTGGCGGGTCCCGCAGCTCTACGCTTCGCCGCCGCGGCTAGCTG
GCAGGTTGTGCGCGGACGCTGCGTGGAACATTTTCCGCGAGTACTGGAGTTTCTGCGATCTCTGCGCGCT
GTTGCCCCTGGCTTGGTTCGCTACCGGCACCACGAACGCCTTTGTATGGGCCTAAAGGCCAAGGTGGTGG
TGGAGCTGATCCTGCAGGGCCGGCCTTGGGCCCAAGTCCTGAAAGCCCTGAATCACCACTTTCCAGAATC
TGGACCTATAGTGCGGGATCCCAAGGCTACAAAGCAGGATCTGAGGAAGATTTTGGAGGCACAGGAAACT
TTTTACCAGCAGGTGAAGCAGCTGTCAGAGGCTCCTGTGGATTTGGCCTCGAAGCTGCAGGAACTTGAAC
AAGAGTATGGGGAACCCTTTCTGGCTGCCATGGAAAAGCTGCTTTTTGAGTACTTGTGTCAGCTGGAGAA
AGCACTGCCTACACCGCAGGCACAGCAGCTTCAGGATGTGCTGAGTTGGATGCAGCCTGGAGTCTCTATC
ACCTCTTCTCTTGCCTGGAGACAATATGGTGTGGACATGGGGTGGCTGCTTCCAGAGTGCTCTGTTACTG
ACTCAGTGAACCTGGCTGAGCCCATGGAACAGAATCCTCCTCAGCAACAAAGACTAGCACTCCACAATCC
CCTGCCAAAAGCCAAGCCTGGCACACATCTTCCTCAGGGACCATCTTCAAGGACGCACCCAGAACCTCTA
GCTGGCCGACACTTCAATCTGGCCCCTCTAGGCCGACGAAGAGTTCAGTCCCAATGGGCCTCCACTAGGG
GAGGCCATAAGGAGCGCCCCACAGTCATGCTGTTTCCCTTTAGGAATCTCGGCTCACCAACCCAGGTCAT
ATCTAAGCCTGAGAGCAAGGAAGAACATGCGATATACACAGCAGACCTAGCCATGGGCACAAGAGCAGCC
TCCACTGGGAAGTCTAAGAGTCCATGCCAGACCCTGGGGGGAAGGGCTCTGAAGGAGAACCCAGTTGACT
TGCCTGCCACAGAGCAAAAGGAGAATTGCTTGGATTGCTACATGGACCCCCTGAGACTATCATTATTACC
TCCTAGGGCCAGGAAGCCAGTGTGTCCTCCGTCTCTGTGCAGCTCCGTCATTACCATAGGGGACTTGGTT
TTAGACTCTGATGAGGAAGAAAATGGCCAGGGGGAAGGAAAGGAATCTCTGGAAAACTATCAGAAGACAA
AGTTTGACACCTTGATACCCACTCTCTGTGAATACCTACCCCCTTCTGGCCACGGTGCCATACCTGTTTC
TTCCTGTGACTGTAGAGACAGTTCTAGACCTTTGTGATAGAACTAAAATGCTCTCTGTACTCTAGTCTCC
TGCCTCCTCAGCTCTGCAAGTAGTTTAGTAGGAATGAAGTGGAAGTCCAGGCTTGGATTGCCTAACTACA
CTGCTAAAAATATTTGTAATCCTTAATAATTAAACTTTGGATTTGTTAAAA
>NM_001363668.2 Homo sapiens TERF1 interacting nuclear factor 2
(TINF2), transcript variant 3, mRNA
  (SEQ ID NO: 13)
GAGGCACCCTCGGGCTCGAGACAGCGGCGACGTTTAAAGCTGAGCGACCCAGTGCCACTGGAGACGGTCA
GCTTCTCCACTCAGGCTCCTCCAGCCCGAGCCAGAAGACCCCCTCCCCCAGAATTCTGGGGGCCGATGGA
AGGGAGCCGAGTCAGATCGCGAGGTACCCAGAGCCGACAGACCGGAGCGACAGGGAGTTGCCAGAAGCCC
CGCCCCTAGGAGTGATCGGAAAGCCTCACCCATCCGGGTGAGGAACCCGGAGGGACCGCCTCCGGGCGGA
GCCCGCCGACCATGGCTACGCCCCTGGTGGCGGGTCCCGCAGCTCTACGCTTCGCCGCCGCGGCTAGCTG
GCAGGTTGTGCGCGGACGCTGCGTGGAACATTTTCCGCGAGTACTGGAGTTTCTGCGATCTCTGCGCGCT
GTTGCCCCTGGCTTGGTTCGCTACCGGCACCACGAACGCCTTTGTATGGGCCTAAAGGCCAAGACAAAGC
AGGATCTGAGGAAGATTTTGGAGGCACAGGAAACTTTTTACCAGCAGGTGAAGCAGCTGTCAGAGGCTCC
TGTGGATTTGGCCTCGAAGCTGCAGGAACTTGAACAAGAGTATGGGGAACCCTTTCTGGCTGCCATGGAA
AAGCTGCTTTTTGAGTACTTGTGTCAGCTGGAGAAAGCACTGCCTACACCGCAGGCACAGCAGCTTCAGG
ATGTGCTGAGTTGGATGCAGCCTGGAGTCTCTATCACCTCTTCTCTTGCCTGGAGACAATATGGTGTGGA
CATGGGGTGGCTGCTTCCAGAGTGCTCTGTTACTGACTCAGTGAACCTGGCTGAGCCCATGGAACAGAAT
CCTCCTCAGCAACAAAGACTAGCACTCCACAATCCCCTGCCAAAAGCCAAGCCTGGCACACATCTTCCTC
AGGGACCATCTTCAAGGACGCACCCAGAACCTCTAGCTGGCCGACACTTCAATCTGGCCCCTCTAGGCCG
ACGAAGAGTTCAGTCCCAATGGGCCTCCACTAGGGGAGGCCATAAGGAGCGCCCCACAGTCATGCTGTTT
CCCTTTAGGAATCTCGGCTCACCAACCCAGGTCATATCTAAGCCTGAGAGCAAGGAAGAACATGCGATAT
ACACAGCAGACCTAGCCATGGGCACAAGAGCAGCCTCCACTGGGAAGTCTAAGAGTCCATGCCAGACCCT
GGGGGGAAGGGCTCTGAAGGAGAACCCAGTTGACTTGCCTGCCACAGAGCAAAAGGAGAATTGCTTGGAT
TGCTACATGGACCCCCTGAGACTATCATTATTACCTCCTAGGGCCAGGAAGCCAGTGTGTCCTCCGTCTC
TGTGCAGCTCCGTCATTACCATAGGGGACTTGGTTTTAGACTCTGATGAGGAAGAAAATGGCCAGGGGGA
AGGAAAGGAATCTCTGGAAAACTATCAGAAGACAAAGTTTGACACCTTGATACCCACTCTCTGTGAATAC
CTACCCCCTTCTGGCCACGGTGCCATACCTGTTTCTTCCTGTGACTGTAGAGACAGTTCTAGACCTTTGT
GATAGAACTAAAATGCTCTCTGTACTCTAGTCTCCTGCCTCCTCAGCTCTGCAAGTAGTTTAGTAGGAAT
GAAGTGGAAGTCCAGGCTTGGATTGCCTAACTACACTGCTAAAAATATTTGTAATCCTTAATAATTAAAC
TTTGGATTTGTTAAAA
>NM_012461.3 Homo sapiens TERF1 interacting nuclear factor 2 (TINF2),
transcript variant 2, mRNA
(SEQ ID NO: 14)
CTCTTACCGCCCTTTTCCGGGGCAAGGGAAGCTAGTAGCGGAGCCGGAAGTGAGGCACCCTCGGGCTCGA
GACAGCGGCGACGTTTAAAGCTGAGCGACCCAGTGCCACTGGAGACGGTCAGCTTCTCCACTCAGGCTCC
TCCAGCCCGAGCCAGAAGACCCCCTCCCCCAGAATTCTGGGGGCCGATGGAAGGGAGCCGAGTCAGATCG
CGAGGTACCCAGAGCCGACAGACCGGAGCGACAGGGAGTTGCCAGAAGCCCCGCCCCTAGGAGTGATCGG
AAAGCCTCACCCATCCGGGTGAGGAACCCGGAGGGACCGCCTCCGGGCGGAGCCCGCCGACCATGGCTAC
GCCCCTGGTGGCGGGTCCCGCAGCTCTACGCTTCGCCGCCGCGGCTAGCTGGCAGGTTGTGCGCGGACGC
TGCGTGGAACATTTTCCGCGAGTACTGGAGTTTCTGCGATCTCTGCGCGCTGTTGCCCCTGGCTTGGTTC
GCTACCGGCACCACGAACGCCTTTGTATGGGCCTAAAGGCCAAGGTGGTGGTGGAGCTGATCCTGCAGGG
CCGGCCTTGGGCCCAAGTCCTGAAAGCCCTGAATCACCACTTTCCAGAATCTGGACCTATAGTGCGGGAT
CCCAAGGCTACAAAGCAGGATCTGAGGAAGATTTTGGAGGCACAGGAAACTTTTTACCAGCAGGTGAAGC
AGCTGTCAGAGGCTCCTGTGGATTTGGCCTCGAAGCTGCAGGAACTTGAACAAGAGTATGGGGAACCCTT
TCTGGCTGCCATGGAAAAGCTGCTTTTTGAGTACTTGTGTCAGCTGGAGAAAGCACTGCCTACACCGCAG
GCACAGCAGCTTCAGGATGTGCTGAGTTGGATGCAGCCTGGAGTCTCTATCACCTCTTCTCTTGCCTGGA
GACAATATGGTGTGGACATGGGGTGGCTGCTTCCAGAGTGCTCTGTTACTGACTCAGTGAACCTGGCTGA
GCCCATGGAACAGAATCCTCCTCAGCAACAAAGACTAGCACTCCACAATCCCCTGCCAAAAGCCAAGCCT
GGCACACATCTTCCTCAGGGACCATCTTCAAGGACGCACCCAGAACCTCTAGCTGGCCGACACTTCAATC
TGGCCCCTCTAGGCCGACGAAGAGTTCAGTCCCAATGGGCCTCCACTAGGGGAGGCCATAAGGAGCGCCC
CACAGTCATGCTGTTTCCCTTTAGGAATCTCGGCTCACCAACCCAGGTCATATCTAAGCCTGAGAGCAAG
GAAGAACATGCGATATACACAGCAGACCTAGCCATGGGCACAAGAGCAGCCTCCACTGGGAAGTCTAAGA
GTCCATGCCAGACCCTGGGGGGAAGGGCTCTGAAGGAGAACCCAGTTGACTTGCCTGCCACAGAGCAAAA
GGAGTGAGTGGAACAGAGTTGCTTCTTACTAGGAGCACATTCTTTGCCTGCCTTCCCTTCATCCTATCCT
CTTTGCTTGCTCTCACCTCAGGAATTGCTTGGATTGCTACATGGACCCCCTGAGACTATCATTATTACCT
CCTAGGGCCAGGAAGCCAGGTAGGTAGTCTGAGTCAGGATTGGATCAACAGCCTCCTCTCTTGGGGACTC
TCAAGAGCCTGTGTTCATCTAGAAGTAGTAGTTTGATTCTGGTTTCCCTCCTACAGTGTGTCCTCCGTCT
CTGTGCAGCTCCGTCATTACCATAGGGGACTTGGTTTTAGACTCTGATGAGGAAGAAAATGGCCAGGGGG
AAGGAAAGGTGAGTGGGAAGGAGCAGAAAGCTGGGAAAGGGGATGGGTAGAACAAGACTGAGAAATCCAC
ATGCTTCAGAATTCAGAGGGTTCAGGGAATGGTTTCGGATAGTAGGCTCTCCCTGCTCCCTTCTCTACAG
GAATCTCTGGAAAACTATCAGAAGACAAAGTTTGACACCTTGATACCCACTCTCTGTGAATACCTACCCC
CTTCTGGCCACGGTGCCATACCTGTTTCTTCCTGTGACTGTAGAGACAGTTCTAGACCTTTGTGATAGAA
CTAAAATGCTCTCTGTACTCTAGTCTCCTGCCTCCTCAGCTCTGCAAGTAGTTTAGTAGGAATGAAGTGG
AAGTCCAGGCTTGGATTGCCTAACTACACTGCTAAAAATATTTGTAATCCTTAATAATTAAACTTTGGAT
TTGTTAAAATAC
```

As used herein, the term “vector” refers to a means of introducing a nucleic acid molecule into a cell, resulting in a transformed cell. Vectors include plasmids, transposons, phages, viruses, liposomes, lipid nanoparticles, and episomes.

By “Viral Protein X (VPX) polypeptide” or “Vpx polypeptide” is meant a protein or fragment thereof having at least 85% amino acid sequence identity to the amino acid sequence of GenBank Accession Nos. P89156 or P18099.1 and having SAM domain and HD domain-containing protein 1 (SAMHD1) binding activity.

```
>sp|P89156|P89156_SIVCZ Vpx protein
[Simian immunodeficiency virus]
  (SEQ ID NO: 15)
MSDPRERIPPGNSGEETIEEAFEWLNRTVEGINRAAVNHLPRELIFQVWQ
RSWEYWHDEMGMSESYTKYRYLCLIQKALFMHCKKGCRCLGEGHGAGGWR
TGPPPPPPPGLA
>sp|P18099.1|VPX_HV2BE [Human immunodeficiency
virus 2]
  (SEQ ID NO: 16)
MTDPRERVPPGNSGEETIGEAFEWLERTIEALNREAVNHLPRELIFQVWQ
RSWRYWHDEQGMSASYTKYRYLCLMQKAIFTHFKRGCTCWGEDMGREGLE
DQGPPPPPPPGLV
```

By “VPX” polynucleotide is meant any nucleic acid molecule encoding a VPX polypeptide or fragment thereof. Exemplary full length sequences of VPX polynucleotides are found below.

VPX (SIV)
(SEQ ID NO: 17)
ATGTCAGATCCCAGGGAGAGAATCCCACCTGGAAACAGTGGAGAAGAGAC
AATAGGAGAGGCCTTCGAATGGCTAAACAGAACAGTAGAGGAGATAAACA
GAGAGGCAGTAAACCACCTACCAAGGGAGCTGATTTTCCAGGTTTGGCAA
AGGTCTTGGGAATACTGGCATGATGAACAAGGGATGTCACAAAGCTATGT
AAAATACAGATACTTGTGTTTAATGCAAAAGGCTTTATTTATGCATTGCA
AGAAAGGCTGTAGATGTCTAGGGGAAGGACACGGGGCAGGAGGATGGAGA
CCAGGACCTCCTCCTCCTCCCCCTCCAGGACTAGCATGA
VPX (HIV2)
(SEQ ID NO: 18)
ATGACAGACCCCAGAGAAAGGGTACCGCCAGGAAACAGTGGAGAAGAGAC
CATTGGAGAGGCCTTCGAGTGGCTAGAGAGGACCATAGAAGCCTTAAACA
GGGAGGCAGTGAACCATCTGCCCCGAGAGCTCATTTTCCAGGTGTGGCAA
AGGTCCTGGAGATATTGGCATGATGAACAAGGGATGTCAGCAAGCTACAC
AAAGTATAGATATTTGTGCCTAATGCAAAAAGCTATATTTACACATTTCA
AGAGAGGGTGCACTTGCTGGGGGGAGGACATGGGCCGGGAAGGATTGGAA
GACCAAGGACCTCCCCCTCCTCCCCCTCCAGGTCTAGTCTAA

Unless specifically stated or obvious from context, as used herein, the term “or” is understood to be inclusive. Unless specifically stated or obvious from context, as used herein, the terms “a”, “an”, and “the” are understood to be singular or plural.

Unless specifically stated or obvious from context, as used herein, the term “about” is understood as within a range of normal tolerance in the art. In some cases, a range of normal tolerance in the art is within 1 or 2 standard deviations of the mean. Unless otherwise clear from context, all numerical values provided herein are modified by the term about.

The recitation of a listing of chemical groups in any definition of a variable herein includes definitions of that variable as any single group or combination of listed groups. The recitation of an embodiment for a variable or aspect herein includes that embodiment as any single embodiment or in combination with any other embodiments or portions thereof.

Any compositions or methods provided herein can be combined with one or more of any of the other compositions and methods provided herein.

### BRIEF DESCRIPTION OF THE DRAWINGS

FIG. 1 provides a schematic representation of modified paired prime editing (“Prime Assembly”) in living cells. Prime editing vectors and double-stranded DNA (dsDNA) or single-stranded DNA (ssDNA) donors with 3′ overhangs are provided. The prime editor is guided by a pair of prime editing guide RNAs (pegRNAs) to synthesize 3′ DNA flaps on opposite DNA strands at a targeted locus of interest. The ssDNA or dsDNA donor anneal to the 3′ flaps via complementary overhangs, the intervening genomic DNA sequence is excised, residual free ssDNA sequences are filled in, and the nicks are ligated, allowing prime assembly of DNA sequences in living cells. The box in the top left panel surrounds the targeted locus of interest/target polynucleotide sequence.

FIGS. 2A and 2B provide a schematic and a graph showing the recoding of a TINF2 dyskeratosis congenita cluster using modified paired prime editing. FIG. 2A is a schematic representation of the dyskeratosis congenita (DC) cluster and the prime editing sites. F4 and R2 indicate the position of the epegRNA spacer sequences. The dsDNA donor or the two ssDNA donors are shown above. Silent point mutations are introduced throughout the DC cluster to decrease the homology between the prime editing flaps and the targeted genomic sequence while restoring the original TINF2 amino acid sequence. FIG. 2B is a prime assembly (PA) and indels quantification as determined by Inference of CRISPR Edits (ICE) and Tracking of Indels by Decomposition (TIDE) analysis from Sanger sequencing. K562 cells were electroporated with Prime Editing (PE) vectors and the indicated concentration of dsDNA or ssDNA donor. Genomic DNA was harvested three days post-nucleofection. n=1 experiment.

FIG. 3 provides a graph showing that phosphorothioate internucleotide linkages enhance modified paired prime editing in K562 cells. The graph shows Prime Assembly (PA) and indels quantification as determined by ICE and TIDE analysis from Sanger sequencing. K562 cells were electroporated with PE vectors and the indicated concentration of dsDNA or ssDNA donor. Where indicated, the donors harbored three phosphorothioate internucleotide linkages between the last four nucleotides (3′ end). Genomic DNA was harvested three days post-nucleofection. n=1 experiment.

FIG. 4 provides a graph showing that decreasing 3′ flap length abrogates modified paired prime editing in K562 cells. The graph shows Prime Assembly (PA) and indels quantification as determined by ICE and TIDE analysis from Sanger sequencing. Flap length is determined by the length of the pegRNA reverse transcriptase template (RTT). K562 cells were electroporated with PE vectors and the indicated concentration of dsDNA or ssDNA donor harboring three phosphorothioate internucleotide linkages between the last four nucleotides (3′ end). Genomic DNA was harvested three days post-nucleofection. n=1 experiment.

FIGS. 5A and 5B provide a schematic and a graph showing efficient modified paired prime editing with short overlaps between donor DNA strands in K562 cells. FIG. 5A is a schematic illustration of the DNA donors harboring different overlap lengths. FIG. 5B is a graph showing Prime Assembly (PA) and indels quantification as determined by ICE and TIDE analysis from Sanger sequencing. K562 cells were electroporated with PE vectors and the indicated concentration of dsDNA or ssDNA donor. DNA donor overlap is indicated as “a”, “b”, “c”, or “d”, following the DNA donor configurations illustrated in FIG. 5A. “-” indicates that no donor was used. Flap length is determined by the length of the pegRNA reverse transcriptase template (RTT). Genomic DNA was harvested three days post-nucleofection. n=1 experiment.

FIGS. 6A-6C provide a schematic, a chart, and a graph showing that twin prime editing allows TINF2 DC cluster recoding in K562 cells. FIG. 6A is a schematic representation of the DC cluster and the twin prime editing (TwinPE) pegRNA sites. F1-F4 and R1-R2 indicate the position of the epegRNA spacer sequences. Silent point mutations are introduced throughout the DC cluster to decrease the homology between the TwinPE flaps and the targeted genomic sequence while restoring the original TIN2 amino acid sequence. The amino acid residues highlighted in gray are residues known to be mutated in DC patients. The most frequently mutated DC cluster residues are highlighted with a star. Figure discloses SEQ ID NO: 7. FIG. 6B is a chart showing the length and percentage of homology of the recoded sequence after TwinPE. The recoded sequence is specified by the RTT sequences of the two pegRNAs. FIG. 6C is a graph showing quantification of the percentage of reads with the specified edit or indels as determined by amplicon sequencing. K562 cells were electroporated with TwinPE vectors and genomic DNA was harvested three days post-nucleofection. n=3 independent biological replicates.

FIGS. 7A-7B are schematics showing components of Prime Editing (FIG. 7A) and the Prime Editing process (FIG. 7B).

FIG. 8 is a schematic showing components of various versions of paired prime editing, and synthesis of two 3′ flaps using the paired prime editing.

FIGS. 9A-9C are a schematic, an image of a gel, and a bar chart showing that prime assembly of four ssDNA fragments allows targeted integration of a U6-pegRNA expression cassette to the ATP1A1 locus. FIG. 9A is a schematic illustration of the targeted U6-pegRNA expression cassette integration at the ATP1A1 locus using four ssDNA donors. The forward pegRNA flap overlap is 25 (v1) or 32 (v2) nucleotides, the overlaps between the ssDNA donors are 28, 32, and 28 nucleotides, and the reverse flap overlap is 32 nucleotides. Prime assembly of the four ssDNA donors allows the integration of a pegRNA expression cassette which expression is driven by the U6 promoter at ATP1A1 intron 17, and simultaneously installs the ATP1A1-T804N gain-of-function mutation at ATP1A1 exon 17, conferring dominant cellular resistance to ouabain. Following prime assembly, marker-free selection with ouabain allows the enrichment of cells stably expressing a pegRNA of interest (or any small RNA of interest). FIG. 9B shows detection of the targeted integration of the U6-pegRNA cassette (B2M-L7stop_v3 pegRNA) at ATP1A1 intron 17 via out-out PCR. K562 cells were electroporated with pCMV-PE7 and standard pegRNA vectors with 800 nM of each ssDNA donor. Three days post-nucleofection, K562 cells were cultured in the presence or absence of 0.5 μM ouabain for 14 days. Genotyping was performed after ouabain selection with primers that bind outside of the targeted region. Representative gel image is from one out of two independent biological replicates. FIG. 9C shows prime editing and indels quantification at B2M as determined by BEAT and TIDE analysis from Sanger sequences. Ouabain-resistant K562 cells stably expressing the B2M-L7stop_v3 pegRNA from FIG. 9B were electroporated with pCMV-PE7 and genomic DNA was harvested three days post-nucleofection. n=2 independent biological replicates. E17, exon 17. UMI, unique molecular identifier.

FIGS. 10A-10D are schematics and bar charts showing that prime assembly allows targeted transgene integration at the TRAC and IL2RG loci without selection. FIG. 10A is a schematic of targeted EGFP integration (1.1 kb) at TRAC via prime assembly using a dsDNA donor with 3′ overhangs. FIG. 10B shows quantification of prime assembly allele with targeted EGFP integration via droplet digital PCR. K562 cells were electroporated with pCMV-PE7, standard pegRNA vectors, and a dsDNA donor with 3′ overhangs. Genomic DNA was harvested three days post-nucleofection. n=3 independent biological replicates. The background level from the donor only control is illustrated with a horizontal dotted line. FIG. 10C is the same as in FIG. 10A for targeted EGFP integration (1.0 kb) at IL2RG. FIG. 10D is the same as in FIG. 10B using flow cytometry quantification of EGFP+ cells seven days post-nucleofection. n=3 independent biological replicates. The background level from the donor only control is illustrated with a horizontal dotted line. SA, splicing acceptor. 2A, 2A self-cleaving peptide. EGFP, enhanced green fluorescent protein. PA, Poly-A signal.

FIGS. 11A-11D are schematics and bar charts showing that prime assembly allows targeted transgene integration at the AAVS1 locus. FIG. 11A is a schematic of targeted EGFP integration at AAVS1 via prime assembly using a dsDNA donor with 3′ overhangs. FIG. 11B shows quantification of targeted EGFP integration (1.1 kb) using flow cytometry. K562 cells were electroporated with pCMV-PE7, standard pegRNA vectors, and a dsDNA donor with 3′ overhangs. The percentage of EGFP-expressing cells was measured seven days post-nucleofection. n=2 independent biological replicates. The background level from the donor only control is illustrated with a horizontal dotted line. FIG. 11C is the same as in FIG. 11A for targeted EGFP integration (1.0 kb) at AAVS1 using a dsDNA donor with 3′ overhangs or ssDNA donors. FIG. 11D is the same as in FIG. 11B. n=3 independent biological replicates. The background level from the donor only control is illustrated with a horizontal dotted line. SA, splicing acceptor. 2A, 2A self-cleaving peptide. EGFP, enhanced green fluorescent protein. PA, Poly-A signal.

### DETAILED DESCRIPTION OF THE INVENTION

The disclosure features systems, compositions, and methods for improved gene editing using a modified paired prime editing system and prime assembly.

The present disclosure is based, at least in part, on the discovery of methods for integrating polynucleotides greater than 100 base pairs in length (e.g., single stranded (ss) DNA or double stranded (ds) DNA donor sequences) into a gene using prime editing guide RNAs (pegRNAs) that target a gene of interest, wherein the pegRNAs feature reverse transcriptase templates that are less than 100% identical (e.g., less than about 85% identical) in sequence to the target polynucleotide. Modified paired prime editing (“Prime Assembly”) improves the versatility and the efficiency of prime editing in living cells to achieve flexible targeted integration and/or insertion of DNA sequences, while advantageously removing any requirement for using long reverse transcriptase template sequences (e.g., >20-25 nt) to effect this integration and/or insertion.

### Prime Editing

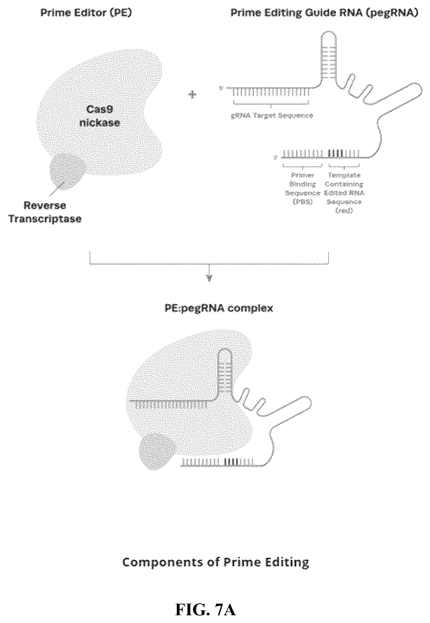

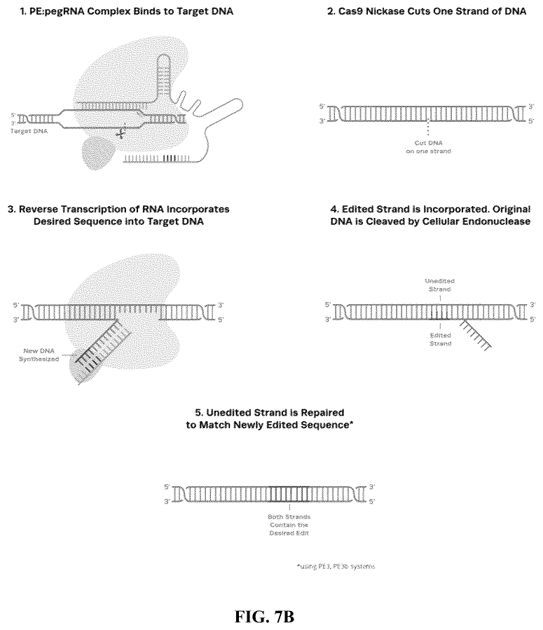

Prime editing is a gene editing method that can alter a target polynucleotide, for example, by targeted insertions, deletions, and base swapping in a precise way. Components of a CRISPR Prime Editing System (shown in FIG. 7A, www.synthego.com/guide/crispr-methods/prime-editing) include a prime editor, which in some embodiments comprises a Cas9 nickase that is fused to a reverse transcriptase, a prime editing guide (peg) polynucleotide (e.g., RNA) that specifies the target site and templates the synthesis of the desired edit in the form of a replacement DNA strand by way of an extension (either DNA or RNA) engineered onto a guide RNA (e.g., at the 5′ or 3′ end, or at an internal portion of a guide RNA). The replacement strand containing the desired edit (e.g., an insertion, deletion, or nucleobase substitution) shares the same sequence as the endogenous strand of the target site to be edited (with the exception that it comprises the desired edit). In embodiments, the pegRNA comprises a gRNA target sequence, also termed a spacer sequence, that hybridizes with a sequence in the DNA target, a primer binding sequence (PBS) and a template containing an edited RNA sequence. In embodiments, a pegRNA comprises the tertiary structure described in FIG. 7A

### Paired Prime Editing

In paired prime editing, two pegRNAs are used (e.g., a first pegRNA and a second pegRNA), with each pegRNA targeting a different DNA strand. The two pegRNAs guide the prime editor comprising a napDNAbp having nickase activity (e.g., Cas9) and a reverse transcriptase to a target sequence, wherein the prime editor nicks the target and synthesizes two 3′ polynucleotide flaps (e.g., a first 3′ polynucleotide flap and a second 3′ polynucleotide flap), as illustrated in FIG. 8. In some embodiments, a single napDNAbp domain and reverse transcriptase is used, whereas in other embodiments, more than one napDNAbp domain and/or reverse transcriptase may be used. Various types of paired prime editing are displayed in FIG. 8.

### Modified Paired Prime Editing (“Prime Assembly”)

Prime assembly is a novel targeted DNA sequence integration technology that provides for efficient integration into a target polynucleotide (e.g., genome) of long donor polynucleotides (e.g., greater than 100 base pairs in length). Prime assembly comprises the use of a paired prime editing system and a donor polynucleotide (e.g., dsDNA or ssDNA) for integration into a target locus, where the paired prime editing system comprises a first pegRNA, a second pegRNA, a nucleic acid programmable DNA binding protein (napDNAbp) (e.g., Cas9) that is fused to or associates with a reverse transcriptase.

In embodiments, the polynucleotide donor sequence comprises between 10 and 3,000 nucleotides. In some embodiments the polynucleotide donor sequence comprises about 5, 10, 20, or 30 kb. In embodiments, the polynucleotide donor sequence may be up to 10 kb. In embodiments, the polynucleotide donor sequence may be up to 1, 2, 3, 4, or 5 kb in length. In embodiments, the polynucleotide donor sequence may be at least about 10, 20, 30, 40, 50, 60, 70, 80, 90, 100, 150, 200, 250, 300, 350, 400, 450, 500, 550, 600, 650, 700, 750, 800, 850, 900, 950, 1000, 1,050, 1,100, 1,150, 1,200, 1,250, 1,300, 1,350, 1,400, 1,450, 1,500, 2,000, 2,500, or 3,000 nucleotides in length.

Each pegRNA comprises a spacer sequence complementary to a target polynucleotide, a primer binding sequence (PBS) and a reverse transcriptase template comprising an edited RNA sequence (e.g., at a 3′ end of the pegRNA). The two pegRNAs guide the prime editor comprising a napDNAbp (e.g., Cas9) and a reverse transcriptase to synthesize two 3′ polynucleotide flaps (e.g., a first 3′ polynucleotide flap and a second 3′ polynucleotide flap), that are each at least partially complementary to the template containing edited RNA sequence of the corresponding pegRNA, as illustrated in FIG. 8. In embodiments, the sequence length of each 3′ polynucleotide flap is between 5-100 nucleotides in length, between 15-40 nucleotides in length, or between 20-25 nucleotides in length. In embodiments, the sequence length of each 3′ nucleotide flap is 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, or 40 nucleotides in length. In embodiments, the 3′ polynucleotide flaps are minimally or not complementary and/or homologous to a sequence of the target locus (e.g., TINF2, the DC cluster, ATP1A1, TRAC, IL2RG, AAVS1). In embodiments, the templates containing edited RNA sequences are minimally or not complementary and/or homologous to a sequence of the target locus (e.g., TINF2, the DC cluster, ATP1A1, TRAC, IL2RG, AAVS1). Without intending to be bound by theory, reducing sequence complementariness and/or homology of the 3′ polynucleotide flaps with the target locus increases the efficiency of prime assembly editing.

The donor sequence may be dsDNA or ssDNA. Where the donor sequence is ssDNA, at least two different ssDNA sequences are used, a first ssDNA sequence, and a second ssDNA sequence, where the first ssDNA sequence and the second ssDNA sequence are at least partially complementary and capable of hybridization, denoted herein as the overlap region. In embodiments, the overlap region is at least 10 nt in length. In embodiments, the overlap region is at least 10-30 nt in length. In embodiments, the overlap region is up to 10 kb or up to 30 kb in length. In embodiments, the overlap region may be up to 1, 2, 3, 4, or 5 kb in length. In embodiments, the overlap region may be up to 150, 200, 250, 300, 350, 400, 450, 500, 550, 600, 650, 700, 750, 800, 850, 900, 950, 1000, 1050, 1100, 1150, 1200, 1250, 1300, 1350, 1400, 1450, 1500, 2000, 2500, or 3000 nt in length. In other embodiments, the overhang overlap length ranges from about 15 nucleotides to about 10 kb (e.g., 15, 20, 25, 30, 40, 50, 75, 100, 250, 500, 1,000, 2,000, 3,000, 5 kb, 10 kb).

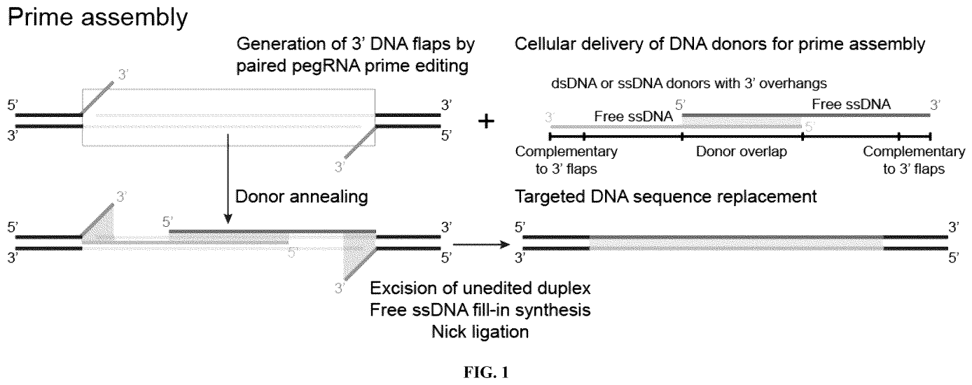

The donor sequence also comprises two 3′ overhang sequences when hybridized (i.e., sequences outside of the overlap region), as illustrated in FIG. 1. In an embodiment, the two 3′ overhang sequences are at least partially complementary and capable of hybridization with a corresponding 3′ polynucleotide flap synthesized by the prime editing system. In some embodiments, a 3′ overhang sequence comprises between 3, 4, 5, 6, 7, 8, 9, 10, 11, 12, 13, 14, 15, 16, 17, 18, 19, or 20 nucleotides. In embodiments, a 3′ overhang sequence is at least 10-30 nt in length. In embodiments, a 3′ overhang sequence is up to 10 kb or up to 30 kb in length. In embodiments, a 3′ overhang sequence region may be up to 1, 2, 3, 4, or 5 kb in length. In embodiments, a 3′ overhang sequence may be up to 150, 200, 250, 300, 350, 400, 450, 500, 550, 600, 650, 700, 750, 800, 850, 900, 950, 1000, 1050, 1100, 1150, 1200, 1250, 1300, 1350, 1400, 1450, 1500, 2000, 2500, or 3000 nucleotides in length. In embodiments, the lengths of the 3′ overhang sequences may be the same or different.

The polynucleotide donor sequence anneals to the 3′ polynucleotide flaps via the complementary 3′ overhang sequences, the intervening genomic DNA sequence is excised, residual ssDNA sequences are filled in, and the nicks are ligated, resulting in integration of the donor sequence at the target locus. The flap length can be about 10, 11, 12, 13, 14, 15, 16, 17, 18, 19, 20, 25, 30, 40, 50 nucleotides.

If desired, multiple donor sequences may be inserted to a genome of a cell or organism. For example, 2, 4, 6, 8, or 10 donor polynucleotides may be inserted.

### Methods of Using Paired Prime Editing

The disclosure provides methods of using paired prime editing and prime assembly to edit a target sequence of interest. Advantageously, this editing can be used to insert long (i.e., greater than 100 nucleotides) donor sequences into a target polynucleotide (e.g., gene, genome), while advantageously removing any requirement for using long reverse transcriptase template sequences (e.g., >20-25 nt) to effect this integration and/or insertion.

### Genome Editing

Prime editing is just one form of gene editing. The paired prime editing and modified paired prime editing (also denoted as “prime assembly”) technology described herein utilizes many of the components used in RNA-guided nuclease-mediated genome editing, based on Type 2 CRISPR (Clustered Regularly Interspaced Short Palindromic Repeat)/Cas (CRISPR Associated) systems. In brief, Cas9, a nuclease guided by single-guide RNA (sgRNA), binds to a targeted genomic locus next to the protospacer adjacent motif (PAM) and generates a double-strand break (DSB). The DSB is then repaired either by non-homologous end joining (NHEJ), which leads to insertion/deletion (indel) mutations, or by homology-directed repair (HDR), which requires an exogenous template and can generate a precise modification at a target locus (Mali et al., Science. 2013 Feb. 15; 339(6121):823-6). Unlike other gene therapy methods, which add a functional, or partially functional, copy of a gene to a patient's cells but retain the original dysfunctional copy of the gene, this system can remove the defect. Genetic correction using engineered nucleases has been demonstrated in tissue culture cells and rodent models of rare diseases.

CRISPR has been used in a wide range of organisms including bakers yeast (S. cerevisiae), zebra fish, nematodes (C. elegans), plants, mice, and several other organisms. Additionally CRISPR has been modified to make programmable transcription factors that allow scientists to target and activate or silence specific genes. Libraries of tens of thousands of guide RNAs are now available.

Since 2012, the CRISPR/Cas system has been used for gene editing (silencing, enhancing or changing specific genes) that even works in eukaryotes like mice and primates. By inserting a plasmid containing cas genes and specifically designed CRISPRs, an organism's genome can be cut at any desired location.

CRISPR repeats range in size from 24 to 48 base pairs. They usually show some dyad symmetry, implying the formation of a secondary structure such as a hairpin, but are not truly palindromic. Repeats are separated by spacers of similar length. Some CRISPR spacer sequences exactly match sequences from plasmids and phages, although some spacers match the prokaryote's genome (self-targeting spacers). New spacers can be added rapidly in response to phage infection.

CRISPR-associated (cas) genes are often associated with CRISPR repeat-spacer arrays. As of 2013, more than forty different Cas protein families had been described. Of these protein families, Cas1 appears to be ubiquitous among different CRISPR/Cas systems. Particular combinations of cas genes and repeat structures have been used to define 8 CRISPR subtypes (Ecoli, Ypest, Nmeni, Dvulg, Tneap, Hmari, Apern, and Mtube), some of which are associated with an additional gene module encoding repeat-associated mysterious proteins (RAMPs). More than one CRISPR subtype may occur in a single genome. The sporadic distribution of the CRISPR/Cas subtypes suggests that the system is subject to horizontal gene transfer during microbial evolution.

Exogenous DNA is apparently processed by proteins encoded by Cas genes into small elements (.about.30 base pairs in length), which are then somehow inserted into the CRISPR locus near the leader sequence. RNAs from the CRISPR loci are constitutively expressed and are processed by Cas proteins to small RNAs composed of individual, exogenously-derived sequence elements with a flanking repeat sequence. The RNAs guide other Cas proteins to silence exogenous genetic elements at the RNA or DNA level. Evidence suggests functional diversity among CRISPR subtypes. The Cse (Cas subtype Ecoli) proteins (called CasA-E in E. coli) form a functional complex, Cascade, that processes CRISPR RNA transcripts into spacer-repeat units that Cascade retains. In other prokaryotes, Cas6 processes the CRISPR transcripts. Interestingly, CRISPR-based phage inactivation in E. coli requires Cascade and Cas3, but not Cas1 and Cas2. The Cmr (Cas RAMP module) proteins found in Pyrococcus furiosus and other prokaryotes form a functional complex with small CRISPR RNAs that recognizes and cleaves complementary target RNAs. RNA-guided CRISPR enzymes are classified as type V restriction enzymes.

See also U.S. Patent Publication 2014/0068797, which is incorporated by reference in its entirety.

### Cas9

Cas9 is a nuclease, an enzyme specialized for cutting DNA, with two active cutting sites, one for each strand of the double helix. The team demonstrated that they could disable one or both sites while preserving Cas9's ability to home located its target DNA. Jinek et al. (2012) combined tracrRNA and spacer RNA into a “single-guide RNA” molecule that, mixed with Cas9, could find and cut the correct DNA targets. It has been proposed that such synthetic guide RNAs might be able to be used for gene editing (Jinek et al., Science. 2012 Aug. 17; 337(6096):816-21).

Cas9 proteins are highly enriched in pathogenic and commensal bacteria. CRISPR/Cas-mediated gene regulation may contribute to the regulation of endogenous bacterial genes, particularly during bacterial interaction with eukaryotic hosts. For example, Cas protein Cas9 of Francisella novicida uses a unique, small, CRISPR/Cas-associated RNA (scaRNA) to repress an endogenous transcript encoding a bacterial lipoprotein that is critical for F. novicida to dampen host response and promote virulence. Coinjection of Cas9 mRNA and sgRNAs into the germline (zygotes) generated mice with mutations. Delivery of Cas9 DNA sequences also is contemplated.

gRNA

As an RNA guided protein, Cas9 utilizes a short RNA to direct the recognition of DNA targets. Though Cas9 preferentially interrogates DNA sequences containing a PAM sequence NGG it can bind here without a protospacer target. However, the Cas9-gRNA complex uses a close match to the gRNA to create a double strand break. CRISPR sequences in bacteria are expressed in multiple RNAs and then processed to create guide strands for RNA. Because Eukaryotic systems lack some of the proteins required to process CRISPR RNAs the synthetic construct gRNA was created to combine the essential pieces of RNA for Cas9 targeting into a single RNA expressed with the RNA polymerase type 2I promoter U6). Synthetic gRNAs are slightly over 100 bp at the minimum length and contain a portion which is targets the 20 protospacer nucleotides immediately preceding the PAM sequence NGG; gRNAs do not contain a PAM sequence.

In one approach, one or more cells of a subject are altered to express a wild-type form of a protein using a CRISPR-Cas system. Cas9 can be used to target a polynucleotide comprising a mutation. Upon target recognition, Cas9 induces double strand breaks in the target gene. Homology-directed repair (HDR) at the double-strand break site can allow insertion of a desired wild-type polynucleotide sequence.

The following US patents and patent publications relating to editing systems are incorporated herein by reference in their entirety: U.S. Pat. No. 8,697,359, 20140170753, 20140179006, 20140179770, 20140186843, 20140186958, 20140189896, 20140227787, 20140242664, 20140248702, 20140256046, 20140273230, 20140273233, 20140273234, 20140295556, 20140295557, 20140310830, 20140356956, 20140356959, 20140357530, 20150020223, 20150031132, 20150031133, 20150031134, 20150044191, 20150044192, 20150045546, 20150050699, 20150056705, 20150071898, 20150071899, 20150071903, 20150079681, 20150159172, 20150165054, 20150166980, and 20150184139.

### Prime Editing Process

Conventional prime editing typically involves the use of a Cas endonuclease and a single guide (sg) RNA to edit sequences without generating a double-stranded break. Paired prime editing and prime assembly utilize many of the components of conventional prime editing. For example, prime editing may use a Cas9 nickase—a variant of Cas9 that nicks the DNA rather than generating double-strand breaks- and a reverse transcriptase having polymerase activity. This combination of a Cas9 and a reverse transcriptase is referred to as a prime editor (PE). The reverse transcriptase may be fused to a Cas9 nickase or untethered from the Cas9 nickase, as shown, for example, by Liu B et al. Nat Biotechnol. 2022 September; 40 (9): 1388-1393 and Grünewald J et al. Nat Biotechnol. 2023 March; 41 (3): 337-343.

At present, multiple versions of prime editors exist. PE1, which is the first version developed, is capable of generating insertions, deletions, and base transversions. PE2 contains certain modifications relative to PEL that led to improved binding and thermostability. PE3 and PE3b include the ability to mend the mismatch sequences that occur with prime editing. See, for example, Huang et al., Front Bioeng Biotechnol. 2023; 11:1039315. PE3 installs another nick on the opposing strand as the nick to which the RT product 3′ flap is appended. PE3b is a subtype of PE3 in which the second nick can only occur after the initial steps of prime editing have occurred. The most recent versions, PE4 and PE5, are versions of PE2 and PE3 respectively in which DNA mismatch repair is inhibited, typically with dominant negative MLH1, to increase the efficiency of prime edit repairs. See, for example, Chen P J et al., Cell. 2021 Oct. 28; 184(22):5635-5652.e29. A variety of twin flap forms of prime editing repair also exist, such as twinPE, Prime Editing-Cas9-based deletion and repair (PEDAR), genome editing by RTTs partially aligned to each other but nonhomologous to target sequences within duo pegRNA (GRAND), PrimeDEL, and bi-direction Prime Editing (Bi-PE). See, for example, Anzalone A V et al., Nat Biotechnol. 2022 May; 40(5):731-740; Choi J et al., Nat Biotechnol. 2022 February; 40 (2): 218-226.

The guide RNA, called prime editing guide RNA (pegRNA), is substantially larger than standard sgRNAs commonly used for CRISPR gene editing (>100 nt vs. 100 nt). The pegRNA is a sgRNA with a primer binding sequence (PBS) and the template containing the desired RNA sequence (reverse transcriptase template, RTT) added at the 3′ end. At present, pegRNAs are created using plasmids, using in-vitro transcription, and by chemical synthesis. Together, the prime editor and the pegRNA form a PE:pegRNA complex, which is used to mediate genome editing within a cell.

A prime editing process is shown in FIG. 7B (www.synthego.com/guide/crispr-methods/prime-editing). The PE:pegRNA complex binds to the target DNA, and Cas9 nicks only one strand, generating a flap. The Primer Binding Sequence, located on the pegRNA, binds to the DNA flap and the reverse transcriptase template is reverse transcribed using the reverse transcriptase. The edited strand is incorporated into the DNA at the end of the nicked flap, and the target DNA is repaired with the new reverse transcribed DNA. The original DNA segment is removed by a cellular endonuclease. This leaves one strand edited, and one strand unedited. In newer PE systems, PE3 and PE3b, the efficiency of the unedited strand being corrected to match the newly edited strand is increased by using an additional standard guide RNA. In this case, the unedited strand is nicked by a Cas9 nickase and the newly edited strand is used as a template to repair the nick, thus completing the edit.

Prime editing is described, for example, in the following references: Anzalone A V, et al. Search-and-replace genome editing without double-strand breaks or donor DNA. Nature. 2019; Zhao D, Li J, Li S, Xin X, Hu M, Price M A, et al. Glycosylase base editors enable C-to-A and C-to-G base changes. Nat. Biotechnol. 2021; 39:35-40; Kurt I C, Zhou R, Iyer S, Garcia S P, Miller B R, Langner L M, et al. CRISPR C-to-G base editors for inducing targeted DNA transversions in human cells. Nat. Biotechnol. 2021; 39:41-6; Chen L, Park J E, Paa P, Rajakumar P D, Prekop H-T, Chew Y T, et al. Programmable C: G to G: C genome editing with CRISPR-Cas9-directed base excision repair proteins. Nat. Commun. 2021; 12:1384; and Liu Y, Li X, He S, Huang S, Li C, Chen Y, et al. Efficient generation of mouse models with the prime editing system. Cell Discov. 2020; 6:1-4.

### Prime Editor

In some embodiments, a prime editor comprises a nucleic acid programable DNA binding protein (napDNAbp) domain that comprises a Cas9 nickase, a Cpf1 nickase, or another CRISPR-Cas nickase. In some embodiments, the napDNAbp comprises a Cas protein domain. In some embodiments, the Cas protein is a Cas9; e.g., Cas9 nuclease; e.g., dCas9, Cas9 nickase.

In some embodiments, the CAS domain is associated with, in complex with, or fused to a reverse transcriptase domain (RT domain), wherein the reverse transcriptase has polymerase activity.

In some embodiments, the prime editor comprises additional polypeptides involved in prime editing, for example, a polypeptide domain having 5′ endonuclease activity, e.g., a 5′ endogenous DNA flap endonucleases (e.g., FEN1), for helping to drive the prime editing process towards the edited product formation. In some embodiments, the prime editor further comprises an RNA-protein recruitment polypeptide, for example, a MS2 coat protein.

In some embodiments, a prime editor comprises a napDNAbp (e.g., Cas) domain and a reverse transcriptase domain having DNA polymerase activity that are derived from different species. For example, a prime editor may comprise a S. pyogenes Cas9 polypeptide and a Moloney murine leukemia virus (M-MLV) reverse transcriptase polypeptide. In some embodiments, the prime editor comprises a fusion polypeptide that comprises a napDNAbp (Cas) domain and a reverse transcriptase domain having polymerase activity that are derived from different species.

Prime Editing Guide RNAs (pegRNA)

In some embodiments, the pegRNA associates with and directs a prime editor to incorporate the one or more intended nucleotide edits into a double stranded target DNA via prime editing. In some embodiments, a pegRNA comprises a spacer sequence that is complementary or substantially complementary to a target sequence, e.g., a target gene. In some embodiments, the pegRNA comprises a gRNA core that associates with a napDNAbp, e.g., a Cas domain, of a prime editor. In some embodiments, the pegRNA further comprises an extended nucleotide sequence comprising one or more intended nucleotide edits compared to a reference sequence, wherein the extended nucleotide sequence may be referred to as an extension arm.

In certain embodiments, the extension arm comprises a primer binding sequence (PBS) that can initiate target-primed DNA synthesis. In some embodiments, the PBS is complementary or substantially complementary to a free 3′ end on the edit strand of the double stranded target DNA, e.g., a target gene at a nick site generated by the prime editor. In some embodiments, the extension arm further comprises a reverse transcriptase template containing an edited RNA sequence that comprises one or more intended nucleotide edits to be incorporated in a target gene by prime editing. In some embodiments, the reverse transcriptase template templates the synthesis of the desired edit by a DNA polymerase domain of the prime editor, for example, a reverse transcriptase domain. The reverse transcriptase template may also be referred to herein as an RT template, or RTT. In some embodiments, the reverse transcriptase template comprises partial complementarity to a target sequence. In some embodiments, the reverse transcriptase template comprises substantial or partial complementarity to the target sequence except at the position of the intended nucleotide edits to be incorporated into the target gene.

In some embodiments, a pegRNA comprises RNA nucleotides. In some embodiments, a pegRNA is a chimeric polynucleotide that comprises both RNA and DNA nucleotides. For example, a pegRNA can include DNA in the spacer sequence, the gRNA core, or the extension arm. In some embodiments, a pegRNA comprises DNA in the spacer sequence. In some embodiments, the entire spacer sequence of a pegRNA is a DNA sequence. In some embodiments, the pegRNA comprises DNA in the gRNA core, for example, in a stem region of the gRNA core. In some embodiments, the pegRNA comprises DNA in the extension arm, for example, in the reverse transcriptase template. A reverse transcriptase template may serve as a DNA synthesis template for a reverse transcriptase having DNA polymerase activity

Components of a pegRNA may be arranged in a modular fashion. In some embodiments, the spacer and the extension arm comprising a primer binding sequence (PBS) and a template containing edited RNA sequence, e.g., a reverse transcriptase template (RTT), can be interchangeably located in the 5′ portion of the pegRNA, the 3′ portion of the pegRNA, or in the middle of the gRNA core. In some embodiments, a pegRNA comprises a PBS and a template containing edited RNA sequence in 5′ to 3′ order. In some embodiments, the gRNA core of a pegRNA may be located in between a spacer and an extension arm of the pegRNA. In some embodiments, the gRNA core of a pegRNA may be located at the 3′ end of a spacer. In some embodiments, the gRNA core of a pegRNA may be located at the 5′ end of a spacer. In some embodiments, the gRNA core of a pegRNA may be located at the 3′ end of an extension arm. In some embodiments, the gRNA core of a pegRNA may be located at the 5′ end of an extension arm. In some embodiments, the pegRNA comprises, from 5′ to 3′: a spacer, a gRNA core, and an extension arm. In some embodiments, the pegRNA comprises, from 5′ to 3′: a spacer, a gRNA core, a reverse transcriptase template, and a PBS. In some embodiments, the pegRNA comprises, from 5′ to 3′: an extension arm, a spacer, and a gRNA core. In some embodiments, the pegRNA comprises, from 5′ to 3′: a reverse transcriptase template, a PBS, a spacer, and a gRNA core.

An intended nucleotide edit in a template containing edited RNA sequence of a pegRNA may comprise various types of alterations as compared to a target gene sequence. In some embodiments, the nucleotide edit is a single nucleotide substitution as compared to the target gene sequence. In some embodiments, the nucleotide edit is a deletion as compared to the target gene sequence. In some embodiments, the nucleotide edit is an insertion as compared to the target gene. In some embodiments, the reverse transcriptase template comprises one to ten intended nucleotide edits as compared to the target gene sequence. In some embodiments, the reverse transcriptase template comprises one or more intended nucleotide edits as compared to the target gene sequence. In some embodiments, the reverse transcriptase template comprises two or more intended nucleotide edits as compared to the target gene sequence. In some embodiments, the reverse transcriptase template comprises three or more intended nucleotide edits as compared to the target gene sequence. In some embodiments, the reverse transcriptase template comprises four or more, five or more, or six or more intended nucleotide edits as compared to the target gene sequence. In some embodiments, the reverse transcriptase template comprises two single nucleotide substitutions, insertions, deletions, or any combination thereof, as compared to the target gene sequence. In some embodiments, the reverse transcriptase template comprises three single nucleotide substitutions, insertions, deletions, or any combination thereof, as compared to the target gene sequence. In some embodiments, the reverse transcriptase template comprises four, five, or six single nucleotide substitutions, insertions, deletions, or any combination thereof, as compared to the target gene sequence. In some embodiments, a nucleotide substitution comprises an adenine (A)-to-thymine (T) substitution. In some embodiments, a nucleotide substitution comprises an A-to-guanine (G) substitution. In some embodiments, a nucleotide substitution comprises an A-to-cytosine (C) substitution. In some embodiments, a nucleotide substitution comprises a T-A substitution. In some embodiments, a nucleotide substitution comprises a T-G substitution. In some embodiments, a nucleotide substitution comprises a T-C substitution. In some embodiments, a nucleotide substitution comprises a G-to-A substitution. In some embodiments, a nucleotide substitution comprises a G-to-T substitution. In some embodiments, a nucleotide substitution comprises a G-to-C substitution. In some embodiments, a nucleotide substitution comprises a C-to-A substitution. In some embodiments, a nucleotide substitution comprises a C-to-T substitution. In some embodiments, a nucleotide substitution comprises a C-to-G substitution.

In some embodiments, a nucleotide insertion is at least 5 nucleotides, at least 6 nucleotides, at least 7 nucleotides, at least 8 nucleotides, at least 9 nucleotides, at least 10 nucleotides, at least 11 nucleotides, at least 12 nucleotides, at least 13 nucleotides, at least 14 nucleotides, at least 15 nucleotides, at least 16 nucleotides, at least 17 nucleotides, at least 18 nucleotides, at least 19 nucleotides, or at least 20 nucleotides in length. In some embodiments, a nucleotide insertion is from 1 to 2 nucleotides, from 1 to 3 nucleotides, from 1 to 4 nucleotides, from 1 to 5 nucleotides, form 2 to 5 nucleotides, from 3 to 5 nucleotides, from 3 to 6 nucleotides, from 3 to 8 nucleotides, from 4 to 9 nucleotides, from 5 to 10 nucleotides, from 6 to 11 nucleotides, from 7 to 12 nucleotides, from 8 to 13 nucleotides, from 9 to 14 nucleotides, from 10 to 15 nucleotides, from 11 to 16 nucleotides, from 12 to 17 nucleotides, from 13 to 18 nucleotides, from 14 to 19 nucleotides, from 15 to 20 nucleotides in length. In some embodiments, a nucleotide insertion is a single nucleotide insertion. In some embodiments, a nucleotide insertion comprises insertion of two nucleotides.

The reverse transcriptase template of a pegRNA may comprise one or more intended nucleotide edits, compared to the target gene, to be edited. Position of the intended nucleotide edit(s) relevant to other components of the pegRNA, or to particular nucleotides (e.g., mutations) in the target gene, may vary. In some embodiments, the nucleotide edit is in a region of the pegRNA corresponding to or homologous to the protospacer sequence. In some embodiments, the nucleotide edit is in a region of the pegRNA corresponding to a region of the double stranded target DNA outside of the protospacer sequence.

In some embodiments, the position of a nucleotide edit incorporation in the target gene may be determined based on position of the protospacer adjacent motif (PAM). For instance, the intended nucleotide edit may be installed in a sequence corresponding to the protospacer adjacent motif (PAM) sequence. In some embodiments, a nucleotide edit in the reverse transcriptase template is at a position corresponding to the 5′ most nucleotide of the PAM sequence. In some embodiments, a nucleotide edit in the reverse transcriptase template is at a position corresponding to the 3′ most nucleotide of the PAM sequence. In some embodiments, position of an intended nucleotide edit in the reverse transcriptase template may be referred to by aligning the reverse transcriptase template with the partially complementary edit strand of the double stranded target DNA, and referring to nucleotide positions on the editing strand where the intended nucleotide edit is incorporated. In some embodiments, a nucleotide edit is incorporated at a position corresponding to about 0, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, or 40 nucleotides upstream of the 5′ most nucleotide of the PAM sequence in the edit strand of the double stranded target DNA. By 0 nucleotide upstream or downstream of a reference position, it is meant that the intended nucleotide is immediately upstream or downstream of the reference position. In some embodiments, a nucleotide edit is incorporated at a position corresponding to about 0 to 2 nucleotides, 0 to 4 nucleotides, 0 to 6 nucleotides, 0 to 8 nucleotides, 0 to 10 nucleotides, 2 to 4 nucleotides, 2 to 6 nucleotides, 2 to 8 nucleotides, 2 to 10 nucleotides, 2 to 12 nucleotides, 4 to 6 nucleotides, 4 to 8 nucleotides, 4 to 10 nucleotides, 4 to 12 nucleotides, 4 to 14 nucleotides, 6 to 8 nucleotides, 6 to 10 nucleotides, 6 to 12 nucleotides, 6 to 14 nucleotides, 6 to 16 nucleotides, 8 to 10 nucleotides, 8 to 12 nucleotides, 8 to 14 nucleotides, 8 to 16 nucleotides, 8 to 18 nucleotides, 10 to 12 nucleotides, 10 to 14 nucleotides, 10 to 16 nucleotides, 10 to 18 nucleotides, 10 to 20 nucleotides, 12 to 14 nucleotides, 12 to 16 nucleotides, 12 to 18 nucleotides, 12 to 20 nucleotides, 12 to 22 nucleotides, 14 to 16 nucleotides, 14 to 18 nucleotides, 14 to 20 nucleotides, 14 to 22 nucleotides, 14 to 24 nucleotides, 16 to 18 nucleotides, 16 to 20 nucleotides, 16 to 22 nucleotides, 16 to 24 nucleotides, 16 to 26 nucleotides, 18 to 20 nucleotides, 18 to 22 nucleotides, 18 to 24 nucleotides, 18 to 26 nucleotides, 18 to 28 nucleotides, 20 to 22 nucleotides, 20 to 24 nucleotides, 20 to 26 nucleotides, 20 to 28 nucleotides, or 20 to 30 nucleotides upstream of the 5′ most nucleotide of the PAM sequence. In some embodiments, the nucleotide edit is incorporated at a position corresponding to 3 nucleotides upstream of the 5′ most nucleotide of the PAM sequence. In some embodiments, the nucleotide edit in is incorporated at a position corresponding to 4 nucleotides upstream of the 5′ most nucleotide of the PAM sequence. In some embodiments, the nucleotide edit is incorporated at a position corresponding to 5 nucleotides upstream of the 5′ most nucleotide of the PAM sequence. In some embodiments, the nucleotide edit in the reverse transcriptase template is at a position corresponding to 6 nucleotides upstream of the 5′ most nucleotide of the PAM sequence.

In some embodiments, an intended nucleotide edit is incorporated at a position corresponding to about 0, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, or 40 nucleotides downstream of the 5′ most nucleotide of the PAM sequence in the edit strand of the double stranded target DNA. In some embodiments, a nucleotide edit is incorporated at a position corresponding to about 0 to 2 nucleotides, 0 to 4 nucleotides, 0 to 6 nucleotides, 0 to 8 nucleotides, 0 to 10 nucleotides, 2 to 4 nucleotides, 2 to 6 nucleotides, 2 to 8 nucleotides, 2 to 10 nucleotides, 2 to 12 nucleotides, 4 to 6 nucleotides, 4 to 8 nucleotides, 4 to 10 nucleotides, 4 to 12 nucleotides, 4 to 14 nucleotides, 6 to 8 nucleotides, 6 to 10 nucleotides, 6 to 12 nucleotides, 6 to 14 nucleotides, 6 to 16 nucleotides, 8 to 10 nucleotides, 8 to 12 nucleotides, 8 to 14 nucleotides, 8 to 16 nucleotides, 8 to 18 nucleotides, 10 to 12 nucleotides, 10 to 14 nucleotides, 10 to 16 nucleotides, 10 to 18 nucleotides, 10 to 20 nucleotides, 12 to 14 nucleotides, 12 to 16 nucleotides, 12 to 18 nucleotides, 12 to 20 nucleotides, 12 to 22 nucleotides, 14 to 16 nucleotides, 14 to 18 nucleotides, 14 to 20 nucleotides, 14 to 22 nucleotides, 14 to 24 nucleotides, 16 to 18 nucleotides, 16 to 20 nucleotides, 16 to 22 nucleotides, 16 to 24 nucleotides, 16 to 26 nucleotides, 18 to 20 nucleotides, 18 to 22 nucleotides, 18 to 24 nucleotides, 18 to 26 nucleotides, 18 to 28 nucleotides, 20 to 22 nucleotides, 20 to 24 nucleotides, 20 to 26 nucleotides, 20 to 28 nucleotides, or 20 to 30 nucleotides downstream of the 5′ most nucleotide of the PAM sequence. In some embodiments, a nucleotide edit is incorporated at a position corresponding to 3 nucleotides downstream of the 5′ most nucleotide of the PAM sequence. In some embodiments, a nucleotide edit is incorporated at a position corresponding to 4 nucleotides downstream of the 5′ most nucleotide of the PAM sequence. In some embodiments, a nucleotide edit is incorporated at a position corresponding to 5 nucleotides downstream of the 5′ most nucleotide of the PAM sequence. In some embodiments, a nucleotide edit is incorporated at a position corresponding to 6 nucleotides downstream of the 5′ most nucleotide of the PAM sequence. By “upstream” and “downstream” it is intended to define relevant positions at least two regions or sequences in a nucleic acid molecule orientated in a 5′-to-3′ direction. For example, a first sequence is upstream of a second sequence in a DNA molecule where the first sequence is positioned 5′ to the second sequence. Accordingly, the second sequence is downstream of the first sequence.

When referred to in the pegRNA, positions of the one or more intended nucleotide edits may be referred to relevant to components of the pegRNA. For example, an intended nucleotide edit may be 5′ or 3′ to the PBS. In some embodiments, a pegRNA comprises the structure, from 5′ to 3′: a spacer, a gRNA core or scaffold, a reverse transcriptase template containing an edited RNA sequence, and a PBS. In some embodiments, the intended nucleotide edit is 0, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, or 40 base pairs upstream to the 5′ most nucleotide of the PBS. In some embodiments, the intended nucleotide edit is 0 to 2 base pairs, 0 to 4 base pairs, 0 to 6 base pairs, 0 to 8 base pairs, 0 to 10 base pairs, 2 to 4 base pairs, 2 to 6 base pairs, 2 to 8 base pairs, 2 to 10 base pairs, 2 to 12 base pairs, 4 to 6 base pairs, 4 to 8 base pairs, 4 to 10 base pairs, 4 to 12 base pairs, 4 to 14 base pairs, 6 to 8 base pairs, 6 to 10 base pairs, 6 to 12 base pairs, 6 to 14 base pairs, 6 to 16 base pairs, 8 to 10 base pairs, 8 to 12 base pairs, 8 to 14 base pairs, 8 to 16 base pairs, 8 to 18 base pairs, 10 to 12 base pairs, 10 to 14 base pairs, 10 to 16 base pairs, 10 to 18 base pairs, 10 to 20 base pairs, 12 to 14 base pairs, 12 to 16 base pairs, 12 to 18 base pairs, 12 to 20 base pairs, 12 to 22 base pairs, 14 to 16 base pairs, 14 to 18 base pairs, 14 to 20 base pairs, 14 to 22 base pairs, 14 to 24 base pairs, 16 to 18 base pairs, 16 to 20 base pairs, 16 to 22 base pairs, 16 to 24 base pairs, 16 to 26 base pairs, 18 to 20 base pairs, 18 to 22 base pairs, 18 to 24 base pairs, 18 to 26 base pairs, 18 to 28 base pairs, 20 to 22 base pairs, 20 to 24 base pairs, 20 to 26 base pairs, 20 to 28 base pairs, or 20 to 30 base pairs upstream to the 5′ most nucleotide of the PBS.

In some embodiments, the pegRNA is an engineered pegRNA (epegRNA), which may also include a stabilizing sequence. In some embodiments, the stabilizing sequence is downstream (i.e., 3′ to) the PBS. Examples of stabilizing sequences include pseudo-knots and/or linker sequences. In an embodiment, the stabilizing sequence has the sequence: CGCGGTTCTATCTAGTTACGCGTTAAACCAACTAGAA (SEQ ID NO: 19). The corresponding positions of the intended nucleotide edit incorporated in the target gene may also be referred to based on the nicking position generated by a prime editor based on sequence homology and complementarity. For example, in embodiments, the distance between the nucleotide edit to be incorporated into the double stranded target DNA, e.g., a target gene, and the nick generated by the prime editor may be determined when the spacer hybridizes with the target sequence and the extension arm hybridizes with the target sequence. In certain embodiments, the position of the nucleotide edit can be in any position downstream of the nick site on the edit strand (or the PAM strand) generated by the prime editor, such that the distance between the nick site and the intended nucleotide edit is 0, 1, 2, 3, 4, 5, 6, 7, 8, 9, 10, 11, 12, 13, 14, 15, 16, 17, 18, 19, 20, 21, 22, 23, 24, 25, 26, 27, 28, 29, or 30 nucleotides in length. In some embodiments, the position of the nucleotide edit is 0, 1, 2, 3, 4, 5, 6, 7, 8, 9, 10, 11, 12, 13, 14, 15, 16, 17, 18, 19, 20, 21, 22, 23, 24, 25, 26, 27, 28, 29, or 30 nucleotides upstream of the nick site on the edit strand. In some embodiments, the position of the nucleotide edit is 0, 1, 2, 3, 4, 5, 6, 7, 8, 9, 10, 11, 12, 13, 14, 15, 16, 17, 18, 19, 20, 21, 22, 23, 24, 25, 26, 27, 28, 29, or 30 nucleotides downstream of the nick site on the edit strand. In some embodiments, the position of the nucleotide edit is 0 base pairs from the nick site on the edit strand, that is, the editing position is at the same position as the nick site. As used herein, the distance between the nick site and the nucleotide edit, for example, where the nucleotide edit comprises an insertion or deletion, refers to the 5′ most position of the nucleotide edit for a nick that creates a 3′ free end on the edit strand (i.e., the “near position” of the nucleotide edit to the nick site). Similarly, as used herein, the distance between the nick site and a PAM position edit, for example, where the nucleotide edit comprises an insertion, deletion, or substitution of two or more contiguous nucleotides, refers to the 5′ most position of the nucleotide edit and the 5′ most position of the PAM sequence.

In some embodiments, the reverse transcriptase template extends beyond a nucleotide edit to be incorporated into the target gene. For example, in some embodiments, the reverse transcriptase template comprises at least 1, 2, 3, 4, 5, 6, 7, 8, 9, 10, 11, 12, 13, 14, 15, 16, 17, 18, 19, 20, 21, 22, 23, 24, 25, 26, 27, 28, 29 or 30 base pairs 3′ to the nucleotide edit to be incorporated to the target gene. In some embodiments, the reverse transcriptase template comprises at least 4 to 30 base pairs 3′ to the nucleotide edit to be incorporated to the target gene. In some embodiments, the reverse transcriptase template comprises at least 4 to 25 base pairs 3′ to the nucleotide edit to be incorporated to the target gene. In some embodiments, the reverse transcriptase template comprises at least 4 to 20 base pairs 3′ to the nucleotide edit to be incorporated to the target gene. In some embodiments, the reverse transcriptase template comprises at least 4 to 30 base pairs 5′ to the nucleotide edit to be incorporated to the target gene. In some embodiments, the reverse transcriptase template comprises at least 4 to 25 base pairs 5′ to the nucleotide edit to be incorporated to the target gene. In some embodiments, the reverse transcriptase template comprises at least 4 to 20 base pairs 5′ to the nucleotide edit to be incorporated to the target gene, sequence.

The reverse transcriptase template of a pegRNA may encode a new single stranded DNA (e.g. by reverse transcription) to replace a target sequence. In some embodiments, the editing target sequence in the edit strand of the double stranded target DNA, e.g., a target gene is replaced by the newly synthesized strand, and the nucleotide edit(s) are incorporated in the region of the double stranded target DNA. In some embodiments, the newly synthesized DNA strand replaces the target sequence. For example, inserted sequences and/or replacement sequences include exon coding sequence replacement, regulatory element insertion (e.g., untranslated region, promoter, enhancer). In some embodiments, the inserted sequence comprises a bar code. Bar codes are useful for clonal tracking. In other embodiments, the inserted sequence encodes a new protein domain, such as a detectable moiety (e.g., epitope tag, fluorescent protein). In other embodiments, the inserted sequence encodes a chimeric antigen receptor (CAR), which finds use in immunotherapy. In some embodiments, incorporation of the newly synthesized DNA strand corrects a mutation present in the target gene or replaces a defective target gene. Such replacement could be by insertion of the donor sequence into the defective gene or by insertion of the donor sequence is into another area of the genome where it can be safely expressed, for example, insertion at a safe harbor locus.

A guide RNA core (also referred to herein as the gRNA core, gRNA scaffold, or gRNA backbone sequence) of a pegRNA may contain a polynucleotide sequence that binds to a napDNAbp (e.g., Cas) of a prime editor. The gRNA core may interact with a prime editor as described herein, for example, by association with a napDNAbp of the prime editor. In some embodiments, the gRNA core or scaffold is a standard spCas9 scaffold, for example, a scaffold with the sequence:

GTTTTAGAGCTAGAAATAGCAAGTTAAAATAAGGCTAGTCCGTTATCAACTTGAAA AAGTGGCACCGAGTCGGTGC (SEQ ID NO: 20). In some embodiments, the gRNA core or scaffold is a F+E spCas9 scaffold sequence, for example, a scaffold with the sequence:

(SEQ ID NO: 21)
GTTTAAGAGCTATGCTGGAAACAGCATAGCAAGTTTAAATAAGGCTAGTC
CGTTATCAACTTGAAAAAGTGGCACCGAGTCGGTGC.

One of skill in the art will recognize that different prime editors having different napDNAbp domains from different DNA binding proteins may use different gRNA core sequences specific to the DNA binding protein. In some embodiments, the gRNA core is capable of binding to a Cas9-based prime editor. In some embodiments, the gRNA core is capable of binding to a Cpf1-based prime editor. In some embodiments, the gRNA core is capable of binding to a Cas12b-based prime editor.

### Pharmaceutical Compositions

Compositions comprising components of paired prime editing systems (e.g., a nucleic acid programmable DNA binding protein having DNA nickase activity fused to or that associates with a reverse transcriptase domain; and two prime editing guide RNAs, each comprising a spacer sequence complementary to the first strand of a double-stranded target polynucleotide sequence, a reverse transcriptase template (also termed a DNA synthesis template), and a primer binding sequence comprising a region complementary to a region upstream of a nick site in the second strand of the double-stranded target DNA sequence) as described herein are provided. In some embodiments, the compositions further comprise a pharmaceutically acceptable carrier, diluent, excipient, or vehicle.

Compositions and preparations (e.g., physiologically or pharmaceutically acceptable compositions) containing gene editing systems (e.g., prime editing, paired prime editing, prime assembly) for parenteral administration include, without limitation, sterile aqueous or non-aqueous solutions, suspensions, and emulsions. Nonlimiting examples of non-aqueous solvents include propylene glycol, polyethylene glycol, vegetable oils, such as olive oil and canola oil, and injectable organic esters, such as ethyl oleate. Aqueous carriers include water, alcoholic/aqueous solutions, emulsions, or suspensions, including saline and buffered media. Parenteral vehicles include, for example, sodium chloride solution, Ringer's dextrose, dextrose and sodium chloride, lactated Ringer's, or fixed oils. Intravenous vehicles include, for example, fluid and nutrient replenishers, electrolyte replenishers (such as those based on Ringer's dextrose), and the like. Preservatives and other additives may also be present in such compositions and preparations, such as, for example, antimicrobials, antioxidants, chelating agents, colorants, stabilizers, inert gases, and the like.

Some of the compositions may potentially be administered as a pharmaceutically acceptable acid- or base-addition salt, formed by reaction with inorganic acids, such as hydrochloric acid, hydrobromic acid, perchloric acid, nitric acid, thiocyanic acid, sulfuric acid, and phosphoric acid, and organic acids, such as formic acid, acetic acid, propionic acid, glycolic acid, lactic acid, pyruvic acid, oxalic acid, malonic acid, succinic acid, maleic acid, and fumaric acid, or by reaction with an inorganic base such as sodium hydroxide, ammonium hydroxide, potassium hydroxide, and organic bases such as mono-, di-, tri-alkyl and aryl amines and substituted ethanolamines.

Provided herein are pharmaceutical compositions which include a therapeutically effective amount of a paired prime editing system or prime assembly system as described herein, alone, or in combination with a pharmaceutically acceptable carrier. Pharmaceutically acceptable carriers include, but are not limited to, saline, buffered saline, dextrose, water, glycerol, ethanol, and combinations thereof. The carrier and composition can be sterile, and the formulation suits the mode of administration. The composition can also contain minor amounts of wetting or emulsifying agents, or pH buffering agents. The composition can be a liquid or aqueous solution, suspension, emulsion, dispersion, tablet, pill, capsule, powder, or sustained release formulation. A liquid or aqueous composition can be lyophilized and reconstituted with a solution or buffer prior to use. The composition can be formulated as a suppository, with traditional binders and carriers such as triglycerides. Oral formulations can include standard carriers, such as pharmaceutical grades of mannitol, lactose, starch, magnesium stearate, sodium saccharine, cellulose, and magnesium carbonate. Any of the commonly known pharmaceutical carriers, such as sterile saline solution or sesame oil, can be used. The medium can also contain conventional pharmaceutical adjunct materials such as, for example, pharmaceutically acceptable salts to adjust the osmotic pressure, buffers, preservatives, and the like. Other media that can be used in the compositions and administration methods as described are normal saline and sesame oil.

### Methods of Treatment

Methods of treating a disease (e.g., a disorder associated with a genetic mutation, such as dyskeratosis congenita (DC)), or symptoms thereof, are provided. In some embodiments, genome editing with a gene editing system (e.g., prime editing, paired prime editing, prime assembly) is carried out on a cell in vitro or in vivo. In other embodiments, methods of treating a disease by genome editing is carried out, for example, using gene editing systems (e.g., prime editing, paired prime editing, prime assembly).

In one embodiment, a cell (e.g., bone marrow cell) of a subject (e.g., a subject having dyskeratosis congenita) is contacted with a gene editing system (e.g., prime editing, paired prime editing, prime assembly), which comprises a Prime Editor and two pegRNAs (e.g., pegRNAs including a template containing edited RNA sequence encoding a wild-type polypeptide or fragment thereof (e.g., wild type TINF2 or a fragment thereof)). In some embodiments, the cell is contacted with the gene editing system (e.g., prime editing, paired prime editing, prime assembly) in vitro. Once the edits have been carried out on the cell in vitro, the cell or a pharmaceutical composition comprising the cell is administered to the subject. Such administration may be by local injection or by system administration (e.g., by infusion).

In other embodiments, a cell of the subject is contacted in vivo with a gene editing system (e.g., prime editing, paired prime editing, prime assembly), which comprises a Prime Editor and two pegRNAs (e.g., pegRNAs including a template containing edited RNA sequence encoding a wild-type polypeptide or fragment thereof (e.g., wild-type TINF2 or a fragment thereof)).

The disclosure provides methods of treating a subject suffering from, or at risk of, or susceptible to disease, or a symptom thereof, or delaying the progression of a disease. In some embodiments, the method comprises administering to the subject (e.g., a mammalian subject), a therapeutic amount of a cell that has been edited according to the methods described herein. In other embodiments, where editing is to take place in vivo, a gene editing system (e.g., prime editing, paired prime editing, prime assembly) is used to contact a cell of the subject in vivo, where the gene editing system comprises a Prime Editor and two pegRNAs (e.g., pegRNAs including a template containing edited RNA sequence encoding a wild-type polypeptide or fragment thereof (e.g., wild-type TINF2 or a fragment thereof)), as described herein.

In some embodiments, the methods herein include administering to the subject (including a human subject identified as in need of such treatment) an effective amount of a gene editing system (e.g., prime editing, paired prime editing, prime assembly), which comprises a Prime Editor and two pegRNAs (e.g., pegRNAs including a template containing edited RNA sequence encoding a wild type polypeptide or fragment thereof (e.g., wild-type TINF2 or a fragment thereof)). The treatment methods are suitably administered to subjects, particularly humans, suffering from, are susceptible to, or at risk of having a disease, or symptoms thereof, namely, any disease treatable by correction of a pathogenic human gene variant by a gene editing system (e.g., prime assembly). In an embodiment, the disease is dyskeratosis congenita.

Identifying a subject in need of such treatment can be based on the judgment of the subject or of a health care professional and can be subjective (e.g., opinion) or objective (e.g., measurable by a test or diagnostic method). Briefly, the determination of those subjects who are in need of treatment or who are “at risk” or “susceptible” can be made by any objective or subjective determination by a diagnostic test (e.g., blood sample, biopsy, genetic test, enzyme or protein marker assay), marker analysis, family history, and the like, including an opinion of the subject or a health care provider. A subject undergoing treatment can be a non-human mammal, such as a veterinary subject, or a human subject (also referred to as a “patient”).

In addition, prophylactic methods of preventing or protecting against a disease (e.g., dyskeratosis congenita), or symptoms thereof, are provided. Such methods comprise administering a cell edited using a gene editing system (e.g., prime editing, paired prime editing, prime assembly), or a therapeutically effective amount of a pharmaceutical composition comprising a gene editing system (e.g., prime editing, paired prime editing, prime assembly), as described herein to a subject (e.g., a mammal, such as a human), in particular, prior to development or onset of a disease.

### Methods of Delivery

Where in vivo editing of cells is contemplated, gene editing systems of the present disclosure (e.g., prime editing, paired prime editing, prime assembly) can be administered to a subject by any of the routes normally used for introducing a recombinant polypeptide. Routes and methods of administration include, without limitation, parenteral, such as intravenous (IV), intradermal, intramuscular, intraperitoneal, intrathecal, or subcutaneous (SC), vaginal, rectal, intranasal, inhalation, intraocular, intracranial, or oral. Parenteral administration, such as subcutaneous, intravenous or intramuscular administration, is generally achieved by injection (immunization). Injectables can be prepared in conventional forms and formulations, either as liquid solutions or suspensions, solid forms (e.g., lyophilized forms) suitable for solution or suspension in liquid prior to injection, or as emulsions. Injection solutions and suspensions can be prepared from sterile powders, granules, and tablets. Administration can be systemic or local.

The gene editing systems (e.g., prime editing, paired prime editing, prime assembly) can be administered in any suitable manner, such as with pharmaceutically acceptable carriers, diluents, or excipients as described supra. Pharmaceutically acceptable carriers are determined in part by the particular composition being administered, as well as by the particular method used to administer the composition. Accordingly, a pharmaceutical composition comprising the gene editing system (e.g., prime editing, paired prime editing, prime assembly), can be prepared using a wide variety of suitable and physiologically and pharmaceutically acceptable formulations. In some embodiments, the disclosed methods include contacting a target DNA sequence (e.g., a pathogenic human gene variant) with a gene editing system (e.g., prime editing, paired prime editing, prime assembly). In some embodiments, the target DNA sequence is a TINF2 polynucleotide sequence, or a fragment thereof. In some embodiments, the target sequence encodes the DC cluster.

Administration of the gene editing systems of the present disclosure (e.g., prime editing, paired prime editing, prime assembly), or pharmaceutical compositions thereof, can be accomplished by single or multiple doses. The dose administered to a subject should be sufficient to induce a beneficial therapeutic response in a subject over time, such as to inhibit, block, reduce, ameliorate, protect against, or prevent disease (e.g., dyskeratosis congenita). The dose required will vary from subject to subject depending on the species, age, weight and general condition of the subject, by the severity of the cancer being treated, by the particular composition being used and by the mode of administration. An appropriate dose can be determined by a person skilled in the art, such as a clinician or medical practitioner, using only routine experimentation. One of skill in the art is capable of determining therapeutically effective amounts of gene editing systems of the present disclosure (e.g., prime editing, paired prime editing, prime assembly), or pharmaceutical compositions thereof, that provide a therapeutic effect or protection against disease (e.g., dyskeratosis congenita) suitable for administering to a subject in need of treatment or protection.

In some embodiments, the gene editing system (e.g., prime editing, paired prime editing, prime assembly), or a pharmaceutical composition thereof, is administered as a maximum-tolerated dose (MTD). In some embodiments, MTD is the dose with estimated probability of dose limiting toxicity (DLT) closest to the target toxicity rate of 20%. In some embodiments, the gene editing system (e.g., prime editing, paired prime editing, prime assembly) or a pharmaceutical composition thereof, is administered in a therapeutically effective dose for a mammal (e.g., human). In some embodiments, the mammal is a mouse. In some embodiments, the mammal is a human.

### Viral Protein X (VPX)

Viral Protein X (VPX) is an accessory gene found in HIV-2 and certain lineages of SIV. VPX is known to antagonize SAM domain and HD domain-containing protein 1 (SAMHD1) by inducing its ubiquitin-proteasome-dependent degradation. SAMHD1 is a gene that was found to restrict HIV-1 from infecting monocyte-derived macrophages (MDM) by hydrolyzing the cellular deoxynucleotide triphosphates (dNTP), reducing their level to below that required for the synthesis of the viral genomic DNA. As a result, VPX has been found to prevent the SAMHD1-mediated decrease in dNTP. In some embodiments, the systems and methods disclosed herein include a VPX polypeptide or polynucleotide encoding a VPX polypeptide. In some embodiments, the systems and methods disclosed herein include additional exogenous deoxynucleosides or dNTPs. In some embodiments, the VPX polypeptide is an HIV-2 VPX polypeptide, an SIV VPX polypeptide, or variants or fragments thereof having activity in mediating the degradation of SAMHD1. In some embodiments, the addition of VPX polypeptide, or polynucleotide encoding a VPX polypeptide, and/or exogenous deoxynucleosides or dNTPs, increases the gene editing efficiency of any of the systems or methods disclosed herein.

### Kits

Also provided are kits containing a gene editing system (e.g., prime editing, paired prime editing, prime assembly), as described herein, and a pharmaceutically acceptable carrier, diluent, or excipient, for administering to a subject, for example. In some embodiments, the kit is provided for treating any disease treatable by correcting a pathogenic gene variant in a subject (e.g., human). In some embodiments, the kit will contain one or more of a gene editing system (e.g., prime editing, paired prime editing, prime assembly), or vectors or other polynucleotides encoding such gene editing systems (e.g., prime editing, paired prime editing, prime assembly), as disclosed herein. As will be appreciated by the skilled practitioner in the art, such a kit may contain one or more containers, labels, carriers, diluents or excipients, as necessary, and instructions for use.

The practice of the present invention employs, unless otherwise indicated, conventional techniques of molecular biology (including recombinant techniques), microbiology, cell biology, biochemistry and immunology, which are well within the purview of the skilled artisan. Such techniques are explained fully in the literature, such as, “Molecular Cloning: A Laboratory Manual”, second edition (Sambrook, 1989); “Oligonucleotide Synthesis” (Gait, 1984); “Animal Cell Culture” (Freshney, 1987); “Methods in Enzymology” “Handbook of Experimental Immunology” (Weir, 1996); “Gene Transfer Vectors for Mammalian Cells” (Miller and Calos, 1987); “Current Protocols in Molecular Biology” (Ausubel, 1987); “PCR: The Polymerase Chain Reaction”, (Mullis, 1994); “Current Protocols in Immunology” (Coligan, 1991). These techniques are applicable to the production of the polynucleotides and polypeptides of the invention, and, as such, may be considered in making and practicing the invention. Useful techniques for particular embodiments will be discussed in the sections that follow.

The following examples are put forth to provide those of ordinary skill in the art with a complete disclosure and description of how to make and use the assay, screening, and therapeutic methods of the invention, and are not intended to limit the scope of what the inventors regard as their invention.

### EXAMPLES

### Example 1: Paired Prime Editing of Hematopoietic Cells for TINF2 Dyskeratosis Congenita

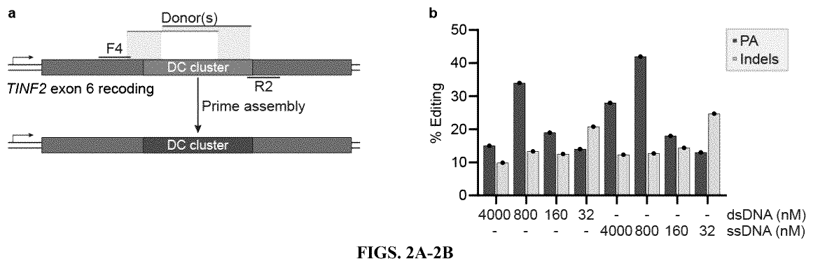

The TINF2 gene encodes for the central component of the shelterin complex, and dominant gain-of-function mutations in a small region of exon 6 encoding for 30 amino acids result in very short telomeres, and the bone marrow failure syndrome dyskeratosis congenita (DC). A mutation-agnostic twin prime editing (TwinPE) method was developed to recode the 30 amino acids region known as the DC cluster (FIG. 2A), which could theoretically correct any pathogenic mutation in the DC cluster associated with DC. Different pairs of engineered prime editing guide RNAs (epegRNAs) were designed and the codons of the DC cluster were optimized to decrease the homology between the prime editing DNA flaps and the targeted genomic locus, allowing highly efficient TwinPE to recode the DC cluster and restore the original TIN2 amino acid sequence.

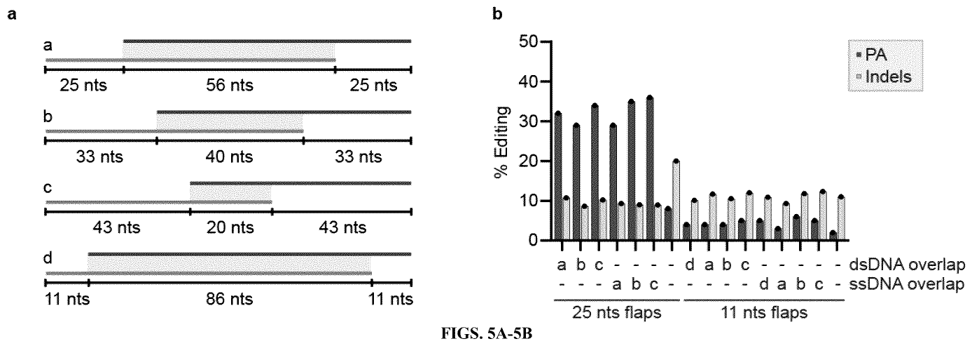

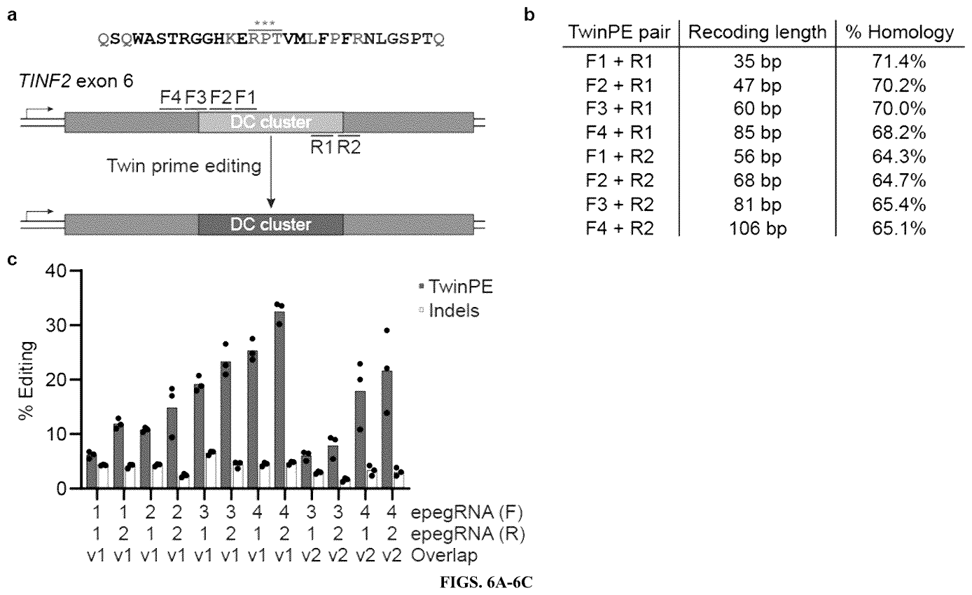

Highly active engineered pegRNA (epegRNAs) to perform Twin Prime Editing at the TINF2 DC cluster were first screened and identified (FIG. 6A). Different engineered pegRNA (epegRNAs) pairs to recode the TINF2 DC cluster were designed and optimized, allowing up to 34% editing via plasmid nucleofection in K562 cells (FIGS. 6A-6C).

### Example 2: Improved Genome Editing Targeted Integration by DNA Sequence Prime Assembly

Conventional prime editing is a genome editing technology that provides for the incorporation of short genetic changes, such as point mutations or short insertions and deletions, in a target polynucleotide. Using a pair of prime editing guide RNAs (pegRNAs) allows the replacement or the integration of larger DNA sequences of up to about 100 base pairs. Longer sequence replacements require ever longer pegRNAs which may be challenging to produce and deliver to target cells.

An approach to assemble and integrate long donor sequences, such as single-stranded DNA (ssDNA) or double-stranded DNA (dsDNA) with 3′ overhangs was devised using a pair of relatively short pegRNAs, that provides for targeted DNA sequence assembly and integration in without requiring double strand breaks (FIG. 1). In brief, a prime editor is guided by a pair of prime editing guide RNAs (pegRNAs) to synthesize 3′ DNA flaps on opposite DNA strands at a targeted locus of interest. Next, a dsDNA with 3′ overhangs or ssDNA donor sequences are introduced which comprises alterations relative to the wild-type sequence. The donor sequences comprise overhangs that include sequences complementary to the 3′ DNA flaps. The ssDNA or dsDNA donor anneals to the 3′ flaps via the complementary overhangs, the intervening genomic DNA sequence is excised, residual ssDNA sequences are filled in, and the nicks are ligated, allowing prime assembly of DNA sequences in living cells. Targeted DNA sequence integration via this strategy, denoted as prime assembly, bypasses the need for long-range reverse transcription and very long prime editing guide RNA reverse transcriptase template sequences or additional recombinase activity, improving the versatility and the efficiency of prime editing in living cells to achieve flexible targeted integration of DNA sequences.

Therapeutic prime assembly was used to recode a TINF2 dyskeratosis congenita cluster (FIGS. 2A-2B). DNA donor(s) and F4 and R2 pegRNA pacer sequences are shown in FIG. 2A. K562 cells were electroporated with Prime Editing (PE) vectors and 32, 160, 800, or 4000 nM of dsDNA or ssDNA donor (FIG. 2B), according to the strategy outlined in FIG. 1.

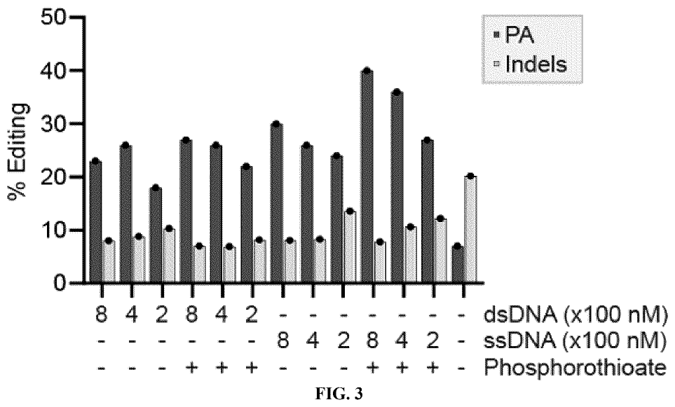

Next, the effects of phosphorothioate internucleotide linkages on prime assembly were explored. Phosphorothioate internucleotide linkages were shown to enhance prime assembly in K562 cells (FIG. 3).

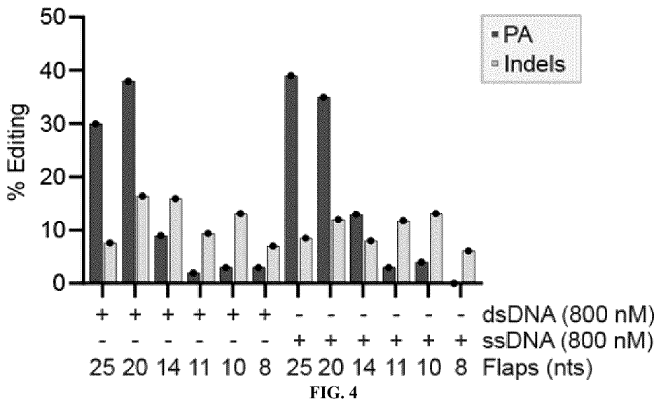

The effects of altering the length of the 3′ DNA flap, as generated by prime editing, was also explored (FIG. 4). It was shown that decreasing 3′ flap length, beneath a certain limit, reduces prime assembly efficiency in K562 cells. The flaps used in these experiments retained partial homology to the endogenous genomic target to retain the exon coding sequence, impeding editing efficiency. In a genome editing context where DNA flaps that share little to no homology to the endogenous genomic target could be used, prime assembly should occur more efficiently.

Finally, the effects of altering the length of DNA overlap were explored (FIGS. 5A-5B). These results show that overlap lengths as short as 20 nts are sufficient to support efficient prime assembly mediated precise sequence replacement. These results also show that the prime assembly donor(s) can be design with a free ssDNA sequence present between the region complementary to the other donor and the region complementary to the 3′ DNA flaps generated by the prime editor, providing design flexibility.

### Example 3: Targeted Insertion of a U6-pegRNA Expression Cassette to the ATP1A1 Locus

Prime assembly was used to insert a U6-pegRNA expression cassete at the ATP1A1 locus (FIG. 9A). The expression cassette included four ssDNA donors with overlapping nucleotide flaps. When integrated, the expression cassette included a U6 promoter and the ATP1A1 T804N gain-of-function mutation that confers dominant cellular resistance to ouabain. The four ssDNA donors were integrated at the ATP1A1 locus using prime assembly, as above, at intron 17 of ATP1A1. Following integration of this expression cassette using prime assembly, ouabain selection allows for the enrichment of cells expression a pegRNA (or any RNA) of interest driven by the U6 promoter, as confirmed by genotyping (FIG. 9B). Prime editing and indels quantification confirmed that efficient editing of the ATP1A1 locus using prime editing was achievable (FIG. 9C).

### Example 4: Targeted Transgene Integration to the TRAC and IL2RG Loci Without Selection

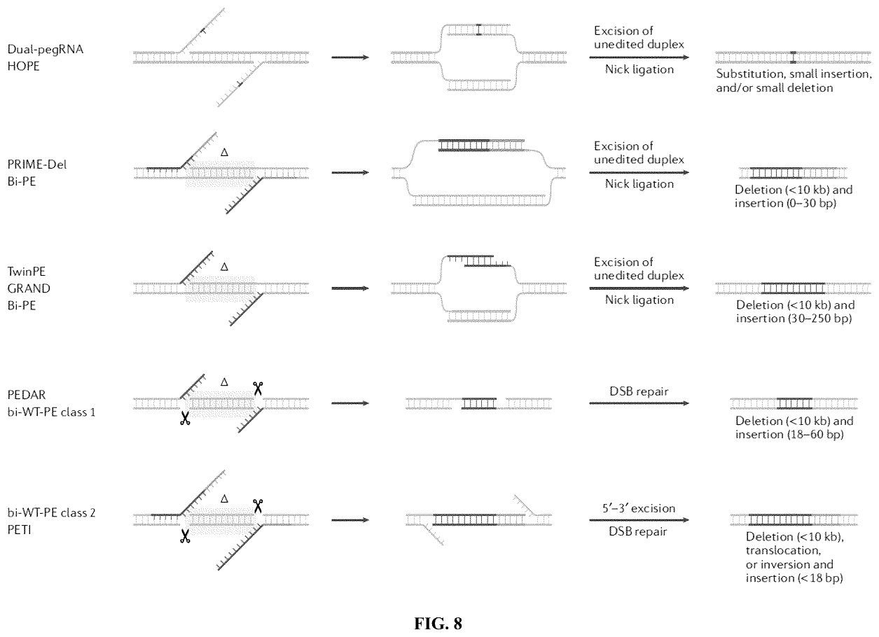

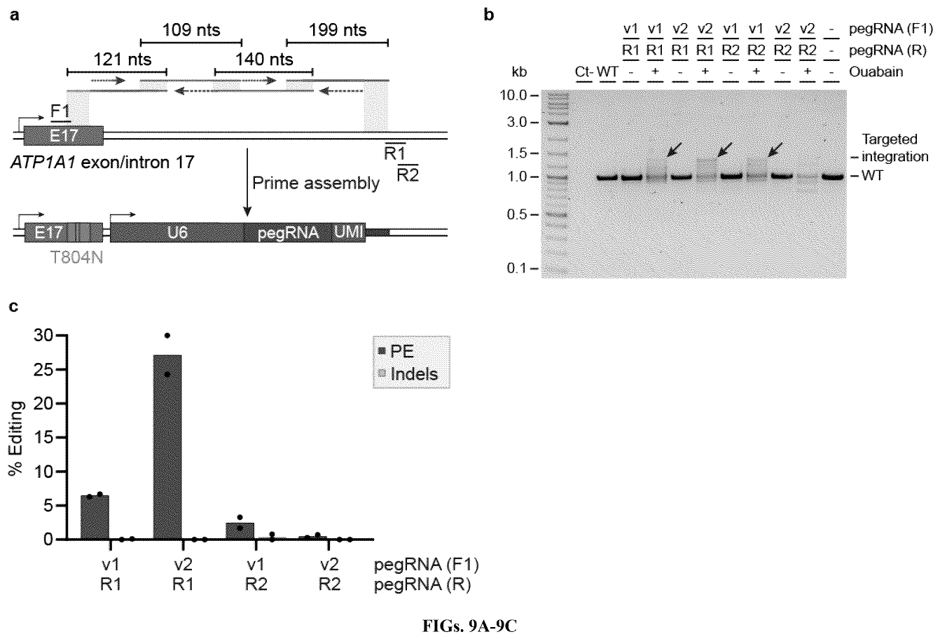

Prime assembly was used to integrate an expression cassette including an EGFP reporter at either the TRAC (FIG. 10A) or IL2RG (FIG. 10C) loci. The expression cassette used was a dsDNA donor sequence with 3′ overhangs. Quantification of prime assembly allele integration was performed for the TRAC locus experiment via droplet digital PCR (FIG. 10B). Quantification of prime assembly allele integration was performed for the IL2RG experiment via flow cytometry quantification of EGFP+ cells (FIG. 10D). Both sets of quantifications demonstrated efficient integration of the prime assembly allele at the TRAC or IL2RG loci, even without the selection of cells.

### Example 5: Prime Assembly Allows Targeted Transgene Integration at the AAVS1 Locus

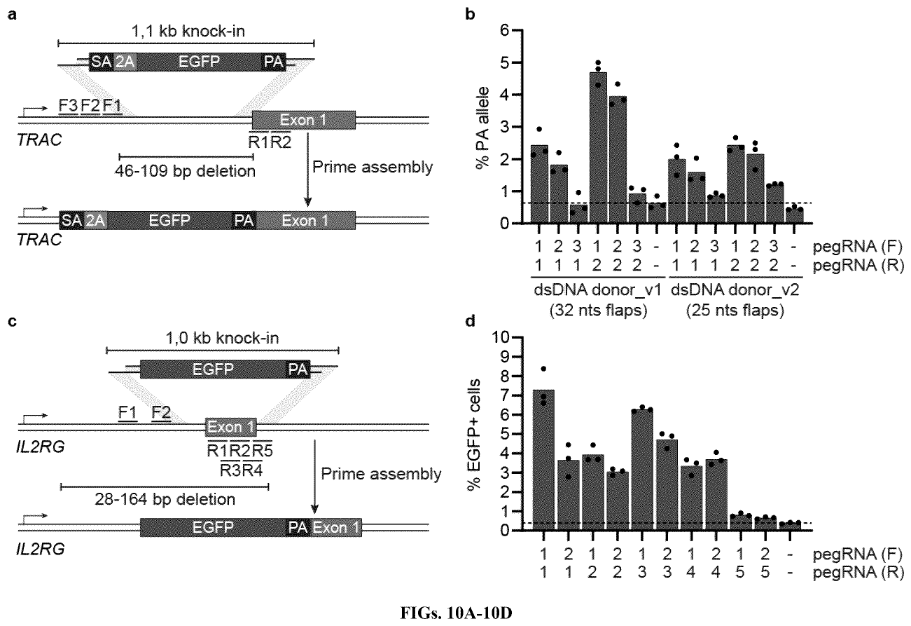

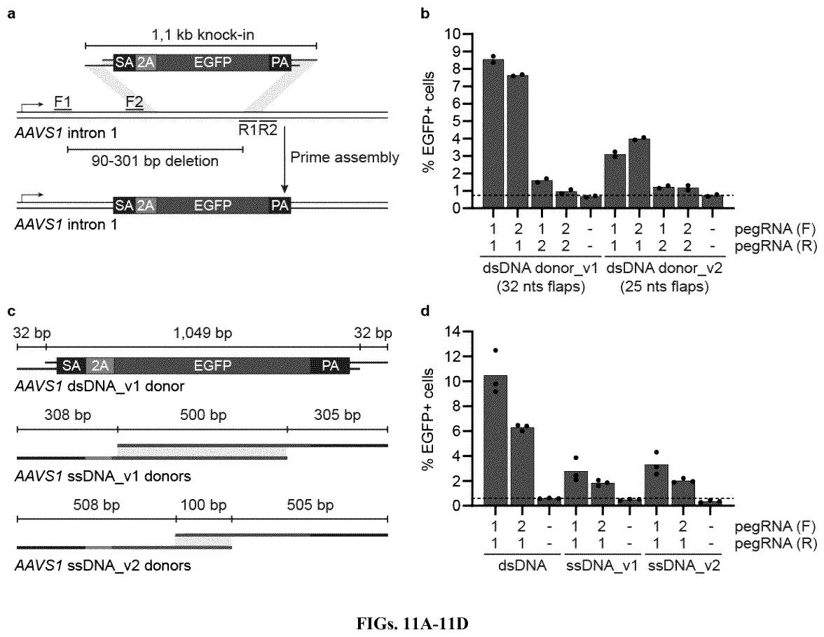

Prime assembly was used to integrate an expression cassette including an EGFP reporter at the AAVS1 locus (FIGS. 11A and 11C). Both ssDNA and dsDNA donor versions of the cassette were tested (FIG. 11C). Quantification of dsDNA donors with 32 nt overlapping flaps and 25 nt overlapping flaps was performed using flow cytometry for EGFP-expressing cells (FIG. 11B). Quantification of a dsDNA donor as compared to ssDNA donors was also performed using flow cytometry for EGFP-expressing cells (FIG. 11D).

The results described hereinabove were carried out using the following methods and materials.

### Methods and Materials

### Cell Culture and Nucleofection

K562 cells were obtained from the ATCC (CCL-243) and cultured at 37° C. under 5% CO₂ in RPMI media supplemented with 10% FBS, and Penicillin/Streptomycin. For each nucleofection, 2×10⁵ cells were electroporated with 750 ng pCMV PEmax (Addgene 174820), 250 ng of each tevopreq1-epegRNA vector (derived from Addgene 174038) harboring the (F+E) scaffold modifications, and the indicated concentration of prime assembly donor with an Amaxa 4D-Nucleofector™ (Lonza) using the SF cell line nucleofection kit (pulse FF-120). Where indicated, K562 cells were electroporated with 750 ng pCMV_PE7 (Addgene 214812) 250 ng of each standard pegRNA vector (derived from Addgene 132777), and the indicated concentration of prime assembly donor. Ouabain octahydrate (Sigma) was dissolved at 5 mg/ml in hot water, working dilutions were prepared in water and stored at −20° C.

### Prime Assembly Donors

Single-stranded DNA (ssDNA) donors were synthesized as ultramers (IDT) at a 4 nmol scale. To generate double-stranded DNA donors, ssDNA ultramers were mixed in 50 mM NaCl, 10 mM Tris-HCl (pH 8.0), 1 mM EDTA, and annealed by heating the solution to 95° C. for 10 minutes, followed by gradual cooling on a thermocycler. The ssDNA and dsDNA donors were then diluted in IDTE buffer and stored at −20° C. Double-stranded DNA (dsDNA) donors with 3′ overhangs were generated via exonuclease digestion. Briefly, donors were cloned in a plasmid vector, and amplified using Kapa-HiFi polymerase (Roche) with primers harboring 5′ phosphorylation, and the expected overhang sequence followed by five consecutive phosphorothioate linkages to block Lambda exonuclease from digesting the donor further. PCR products were purified using SPRIselect beads (Beckman Coulter), digested with Lambda exonuclease (NEB), and purified again using SPRIselect beads. Donor concentration and purity was assessed by nanodrop.

For experiments requiring long ssDNA, donors were amplified using Kapa-HiFi polymerase with a primer harboring a 5′ biotin modification for the DNA strand to separate, and a primer harboring a 5′ phosphorylation for the DNA strand to isolate. PCR amplicons were purified with SPRIselect beads. The single strand of interest was then purified via magnetic separation using Streptavidin C1 Dynabeads. Briefly, Streptavidin C1 Dynabeads were washed two times, mixed with biotinylated PCR amplicons, and incubated at room temperature for 30 minutes with agitation. For magnetic separation, Dynabeads coated with biotinylated amplicons were washed twice, and the supernatant was removed and replaced with 50 μl 0.125 M NaOH melt solution (prepared fresh) to denature the dsDNA. The solution was placed back on the magnet and the supernatant containing the nonbiotinylated strand was removed gently and mixed immediately with Neutralization buffer (freshly prepared by mixing 200 μl 3M sodium acetate pH 5.2 with 4.8 ml 1×TE buffer). A second round of denaturation and elution was performed with 50 μl 0.125 M NaOH melt solution. Resulting ssDNA was purified using SPRIselect beads, and ssDNA concentration and purity was assessed by nanodrop.

### Genotyping

Genomic DNA was harvested 3 days post-nucleofection using QuickExtract DNA extraction solution (Epicentre) following manufacturer's recommendations. PCR amplifications were performed with 30 cycles of amplification with Phusion high-fidelity polymerase. The percentage of prime assembly alleles and indels were quantified using ICE and TIDE webtools from Sanger sequence data files, respectively. For prime editing at B2M, the percentage of prime edited alleles and indels were quantified using BEAT and TIDE webtools from Sanger sequence data files, respectively.

### Droplet Digital PCR

Genomic DNA was extracted and purified using the DNeasy Blood and Tissue Kit (Qiagen). For each ddPCR assay, 50 ng of genomic DNA was used. The droplets were generated using a Bio-Rad QX200 AutoDG droplet digital PCR system with ddPCR supermix (no dUTP) (Bio-Rad), and HindIII-HF was supplemented (NEB) in each reaction. Following droplet generation, samples were amplified using the following conditions: 95° C. for 10 min, 40 cycles of 94° C. for 30 s and 56° C. for 60 s, and a final incubation at 98° C. for 10 min. Samples were then kept at 4° C. until analysis. Results were analyzed using the QuantaSoft software, and the percentage of prime assembly (PA) alleles harboring the targeted transgene integration at TRAC (chromosome 14) was determined as the ratio of PA allele relative to the genomic reference AKT1 on chromosome 14 (Bio-Rad, Assay ID: dHsaCP2506960).

### Flow Cytometry

The percentage of fluorescent cells was quantified using a BD LSRII flow cytometer, and 1×10⁵ cells were analyzed for each condition. Cells were cultured for seven days post-nucleofection, and donor only conditions were used as a negative control. Flow cytometric data visualization and analysis was performed using FlowJo (v10).

TABLE 1
Engineered pegRNA (epegRNA) Sequences Used in Example 1.

| TINF2 | SEQ ID | | SEQ ID | |
|---|---|---|---|---|
| epegRNA | NO: | Spacer | NO: | Extension |
| F1_v1 | 22 | GTCCACTAG | 28 | GAATGGGAAGAGCATCACGGTAGGCCGT |
| | | GGGAGGCCA | | TCCTTGTGGCCTCCCCTAGT |
| | | TA | | |
| F2_v1 | 23 | GTCCCAATG | 29 | GAATGGGAAGAGCATCACGGTAGGCCGT |
| | | GGCCTCCAC | | TCCTTGTGTCCGCCTCTGGTGGAGGCCC |
| | | TA | | ATTG |
| F3_v1 | 24 | GACGAAGAG | 30 | GAATGGGAAGAGCATCACGGTAGGCCGT |
| | | TTCAGTCCC | | TCCTTGTGTCCGCCTCTGGTGCTAGCCCA |
| | | AA | | CTGGGACTGAACTCTT |
| F4_v1 | 25 | GACTTCAAT | 31 | GAATGGGAAGAGCATCACGGTAGGCCGT |
| | | CTGGCCCCT | | TCCTTGTGTCCGCCTCTGGTGCTAGCCCA |
| | | CT | | CTGGCTCTGCACTCTCCGTCTTCCCAATG |
| | | | | GGGCCAGATTGA |
| F3_v2 | 24 | GACGAAGAG | 32 | GTTCCTTGTGTCCGCCTCTGGTGCTAGCC |
| | | TTCAGTCCC | | CACTGGGACTGAACTCTT |
| | | AA | | |
| F4_v2 | 25 | GACTTCAAT | 33 | GTTCCTTGTGTCCGCCTCTGGTGCTAGCC |
| | | CTGGCCCCT | | CACTGGCTCTGCACTCTCCGTCTTCCCAA |
| | | CT | | TGGGGCCAGATTGA |
| R1_v1 | 26 | GGTGAGCCG | 34 | GCCTACCGTGATGCTCTTCCCATTCAGGA |
| | | AGATTCCTAA | | ATCTCGGCT |
| | | A | | |
| R2_v1 | 27 | GGCTTAGATA | 35 | GCCTACCGTGATGCTCTTCCCATTCCGCA |
| | | TGACCTGGG | | ACCTGGGAAGCCCTACACAGGTCATATC |
| | | T | | TA |
| R1_v2 | 26 | GGTGAGCCG | 36 | GCTAGCACCAGAGGCGGACACAAGGAA |
| | | AGATTCCTAA | | CGGCCTACCGTGATGCTCTTCCCATTCAG |
| | | A | | GAATCTCGGCT |
| R2_v2 | 27 | GGCTTAGATA | 37 | GCTAGCACCAGAGGCGGACACAAGGAA |
| | | TGACCTGGG | | CGGCCTACCGTGATGCTCTTCCCATTCCG |
| | | T | | CAACCTGGGAAGCCCTACACAGGTCAT |
| | | | | ATCTA |

The primer binding sequence (PBS) is underlined and highlighted in bold. T indicates the presence of uracil.

TABLE 2
Engineered pegRNA (epegRNA) Sequences Used in Example 2.

| TINF2 | SEQ ID | | SEQ ID | |
|---|---|---|---|---|
| epegRNA | NO: | Spacer | NO: | Extension |
| F4_25nts | 25 | GACTTCAATCTGGC | 38 | GCTCTGCACTCTCCGTCTTCCCA |
| | | CCCTCT | | ATGGGGCCAGATTGA |
| F4_20nts | 25 | GACTTCAATCTGGC | 39 | GCACTCTCCGTCTTCCCAATGG |
| | | CCCTCT | | GGCCAGATTGA |
| F4_14nts | 25 | GACTTCAATCTGGC | 40 | TCCGTCTTCCCAATGGGGCCAG |
| | | CCCTCT | | ATTGA |
| F4_11nts | 25 | GACTTCAATCTGGC | 41 | GTCTTCCCAATGGGGCCAGATT |
| | | CCCTCT | | GA |
| F4_10nts | 25 | GACTTCAATCTGGC | 42 | TCTTCCCAATGGGGCCAGATT |
| | | CCCTCT | | GA |
| F4_8nts | 25 | GACTTCAATCTGGC | 43 | TTCCCAATGGGGCCAGATTGA |
| | | CCCTCT | | |
| R2_25nts | 27 | GGCTTAGATATGACC | 44 | ATTCCGCAACCTGGGAAGCCCT |
| | | TGGGT | | ACACAGGTCATATCTA |
| R2_20nts | 27 | GGCTTAGATATGACC | 45 | GCAACCTGGGAAGCCCTACACA |
| | | TGGGT | | GGTCATATCTA |
| R2_14nts | 27 | GGCTTAGATATGACC | 46 | TGGGAAGCCCTACACAGGTCA |
| | | TGGGT | | TATCTA |
| R2_11nts | 27 | GGCTTAGATATGACC | 47 | GAAGCCCTACACAGGTCATATC |
| | | TGGGT | | TA |
| R2_10nts | 27 | GGCTTAGATATGACC | 48 | AAGCCCTACACAGGTCATATCT |
| | | TGGGT | | A |
| R2_8nts | 27 | GGCTTAGATATGACC | 49 | GCCCTACACAGGTCATATCTA |
| | | TGGGT | | |

The primer binding sequence (PBS) is underlined and highlighted in bold.
T indicates the presence of uracil.

TABLE 3
Ultramer Donor Sequences Used.

| Ultramer | SEQ ID NO: | Sequence |
|---|---|---|
| TINF2- | 50 | CAGTGGGCTAGCACCAGAGGCGGACACAAGGAACGGCCTACCG |
| PA_Top_a | | TGATGCTCTTCCCATTCCGCAACCTGGGAAGCCCTACA |
| TINF2- | 51 | GGGAAGAGCATCACGGTAGGCCGTTCCTTGTGTCCGCCTCTGGT |
| PA Bot a | | GCTAGCCCACTGGCTCTGCACTCTCCGTCTTCCCAAT |
| TINF2- | 52 | CAGTGGGCTAGCACCAGAGGCGGACACAAGGAACGGCCTACCG |
| PA_Top_ | | TGATGCTCTTCCCATTCCGCAACCTGGGAAGCCCT*A*C*A |
| a_PT | | |
| TINF2- | 53 | GGGAAGAGCATCACGGTAGGCCGTTCCTTGTGTCCGCCTCTGGT |
| PA_Bot_ | | GCTAGCCCACTGGCTCTGCACTCTCCGTCTTCCC*A*A*T |
| a_PT | | |
| TINF2- | 54 | TAGCACCAGAGGCGGACACAAGGAACGGCCTACCGTGATGCTCT |
| PA_Top_b | | TCCCATTCCGCAACCTGGGAAGCCCTACA |
| TINF2- | 55 | CATCACGGTAGGCCGTTCCTTGTGTCCGCCTCTGGTGCTAGCCCA |
| PA_Bot b | | CTGGCTCTGCACTCTCCGTCTTCCCAAT |
| TINF2- | 56 | GGCGGACACAAGGAACGGCCTACCGTGATGCTCTTCCCATTCCG |
| PA_Top_c | | CAACCTGGGAAGCCCTACA |
| TINF2- | 57 | GGCCGTTCCTTGTGTCCGCCTCTGGTGCTAGCCCACTGGCTCTGC |
| PA_Bot_c | | ACTCTCCGTCTTCCCAAT |
| TINF2- | 58 | GGAGAGTGCAGAGCCAGTGGGCTAGCACCAGAGGCGGACACAA |
| PA_Top_d | | GGAACGGCCTACCGTGATGCTCTTCCCATTCCGCAACCTGGGAA |
| | | GCCCTACA |
| TINF2- | 59 | CCAGGTTGCGGAATGGGAAGAGCATCACGGTAGGCCGTTCCTTG |
| PA_Bot_d | | TGTCCGCCTCTGGTGCTAGCCCACTGGCTCTGCACTCTCCGTCTT |
| | | CCCAAT |
| ATP1A1- | 60 | GCATATACGATACAAGGCTGTTAGAGAGATAATTAGAATTAATTTGACTGTAAACA |
| PA_U6_ | | CAAAGATATTAGTACAAAATACGTGACGTAGAAAGTAATAATTTCTTGGG*T*A*G |
| Top-1 | | |
| ATP1A1- | 61 | CTCTCTAACAGCCTTGTATCGTATATGCAAATATGAAGGAATCATGGGAAATAGGC |
| PA_T804N_ | | CCTCTGTTGTGACACTCACCATGTCAGTGCCCAGATCGATACACAAAATTGTCACG |
| Bot-1 | | TTGCCC*A*A*A |
| ATP1A1- | 62 | ATATATCTTGTGGAAAGGACGAAACACCGAGTAGCGCGAGCACAGCTAGTTTTA |
| PA_B2M_peg | | GAGCTAGAAATAGCAAGTTAAAATAAGGCTAGTCCGTTATCAACTTGAAAAAGTG |
| RNA_Top-2 | | GCACCGAGTCGGTGCTCCGTGGGGTGAGCTGTGCTCGCGCTTTTTTTTNNNNN |
| | | NNNNNGCTAGCATCAGAGTTGGACACATGGAACG*T*T*G |
| ATP1A1- | 63 | GGTGTTTCGTCCTTTCCACAAGATATATAAAGCCAAGAAATCGAAATACTTTCAAG |
| PA_U6_ | | TTACGGTAAGCATATGATAGTCCATTTTAAAACATAATTTTAAAACTGCAAACTACC |
| Bot-2 | | CAAGAAATTATTACTTTCTACGTC*A*C*G |

Phosphorothioate internucleotide linkages are annotated as (*).

TABLE 4
Scaffold and Stabilizing Sequences used.

| | | |
|---|---|---|
| Standard SpCas9 | SEQ ID | GTTTTAGAGCTAGAAATAGCA |
| sgRNA scaffold | NO: 20 | AGTTAAAATAAGGCTAGTCCG |
| sequence | | TTATCAACTTGAAAAAGTGGC |
| | | ACCGAGTCGGTGC |
| F+E SpCas9 sgRNA | SEQ ID | GTTTAAGAGCTATGCTGGAAA |
| scaffold sequence | NO: 21 | CAGCATAGCAAGTTTAAATAA |
| | | GGCTAGTCCGTTATCAACTTG |
| | | AAAAAGTGGCACCGAGTCGGT |
| | | GC |
| tevopreQ1 | SEQ ID | CGCGGTTCTATCTAGTTACGC |
| structural motif | NO: 19 | GTTAAACCAACTAGAA |
| (stabilizing | | |
| sequence) | | |

T indicates the presence of uracil.

TABLE 5
Standard pegRNA sequences used.

| | | | | |
|---|---|---|---|---|
| | SEQ ID | | SEQ ID | |
| pegRNA | NO: | Spacer | NO: | Extension |

ATP1A1 pegRNAs

| | | | | |
|---|---|---|---|---|
| F1 v1 | 64 | GCAAACATTCCACTAC | 83 | ACACAAAATTGTCACGTTGCCCA |
| | | CACT | | AAGGTAGTGGAATGT |
| F1 v2 | 64 | GCAAACATTCCACTAC | 84 | GATCGATACACAAAATTGTCACG |
| | | CACT | | TTGCCCAAAGGTAGTGGAATGT |
| R1 | 65 | GCGCCAGCCTCATGG | 85 | GCTAGCATCAGAGTTGGACACAT |
| | | ATGCT | | GGAACGTTGATCCATGAGGCTG |
| R2 | 66 | GTTTCCCAACGCCAG | 86 | GCTAGCATCAGAGTTGGACACAT |
| | | CCTCA | | GGAACGTTGGGCTGGCGTTG |

TRAC pegRNAs

| | | | | |
|---|---|---|---|---|
| F1_v1 | 67 | GCCTGGGTTGGGGCA | 87 | GCTGGTCATTGCGGTCTCATTGG |
| | | AAGA | | TGTACGGTATTGCCCCAACC |
| F1 v2 | 67 | GCCTGGGTTGGGGCA | 88 | GTTCCGGTCTCATTGGTGTACGG |
| | | AAGA | | TATTGCCCCAACC |
| F2 v1 | 68 | GCTTGTCCATCACTGG | 89 | GCTGGTCATTGCGGTCTCATTGG |
| | | CATC | | TGTACGGTAGCCAGTGATGGA |
| F2 v2 | 68 | GCTTGTCCATCACTGG | 90 | GTTCCGGTCTCATTGGTGTACGG |
| | | CATC | | TAGCCAGTGATGGA |
| F3_v1 | 69 | GCCCCGCCCTTGTCCA | 91 | GCTGGTCATTGCGGTCTCATTGG |
| | | TCAC | | TGTACGGTAATGGACAAGGGC |
| F3 v2 | 69 | GCCCCGCCCTTGTCCA | 92 | GTTCCGGTCTCATTGGTGTACGG |
| | | TCAC | | TAATGGACAAGGGC |
| R1 v1 | 70 | GTCAGGGTTCTGGATA | 93 | GCTAGCATCAGAGTTGGACACAT |
| | | TCTGT | | GGAACGTTGGATATCCAGAACC |
| R1 v2 | 70 | GTCAGGGTTCTGGATA | 94 | GCCTACCGTGATGCTCTTCCCAT |
| | | TCTGT | | TCGATATCCAGAACC |
| R2 v1 | 71 | GAGTCTCTCAGCTGG | 95 | GCTAGCATCAGAGTTGGACACAT |
| | | TACA | | GGAACGTTGACCAGCTGAGAG |
| R2_v2 | 71 | GAGTCTCTCAGCTGG | 96 | GCCTACCGTGATGCTCTTCCCAT |
| | | TACA | | TCACCAGCTGAGAG |

IL2RG pegRNAs

| | | | | |
|---|---|---|---|---|
| F1 | 72 | GGGTAGTGGGTGAGG | 97 | GCTGGTCTATGCGGTCTCTAAGG |
| | | GACCC | | TGTACGGTATCCCTCACCCA |
| F2 | 73 | GACACAGACAGACTA | 98 | GCTGGTCTATGCGGTCTCTAAGG |
| | | CACCC | | TGTACGGTATGTAGTCTGTCTG |
| R1 | 74 | GGTAATGATGGCTTCA | 99 | GCTAGCATCAGAGTTGGACACAT |
| | | ACA | | GGAACGTTGTGAAGCCATCATT |
| R2 | 75 | GGAATAAGAGGGATG | 100 | GCTAGCATCAGAGTTGGACACAT |
| | | TGAA | | GGAACGTTGACATCCCTCTTATT |
| R3 | 76 | GGGCAGCTGCAGGAA | 101 | GCTAGCATCAGAGTTGGACACAT |
| | | TAAGA | | GGAACGTTGTATTCCTGCAGCT |
| R4 | 77 | GTTCAGCCCCACTCCC | 102 | GCTAGCATCAGAGTTGGACACAT |
| | | AGCA | | GGAACGTTGTGGGAGTGGGG |
| R5 | 78 | GCCAGATTTCCCACCA | 103 | GCTAGCATCAGAGTTGGACACAT |
| | | GCTG | | GGAACGTTGCTGGTGGGAAAT |

AAVS1 pegRNAs

| | | | | |
|---|---|---|---|---|
| F1 v1 | 79 | GGGGCCACTAGGGAC | 104 | GCTGGTCATTGCGGTCTCATTGG |
| | | AGGAT | | TGTACGGTACTGTCCCTAGTG |
| F1 v2 | 79 | GGGGCCACTAGGGAC | 105 | GTTCCGGTCTCATTGGTGTACGG |
| | | AGGAT | | TACTGTCCCTAGTG |
| F2 v1 | 80 | GATGGAGCCAGAGAG | 106 | GCTGGTCATTGCGGTCTCATTGG |
| | | GATCC | | TGTACGGTATCCTCTCTGGC |
| F2 v2 | 80 | GATGGAGCCAGAGAG | 107 | GTTCCGGTCTCATTGGTGTACGG |
| | | GATCC | | TATCCTCTCTGGC |
| R1 v1 | 81 | GCAGCTCAGGTTCTG | 108 | GCTAGCATCAGAGTTGGACACAT |
| | | GGAGA | | GGAACGTTGCCCAGAACC |
| R1 v2 | 81 | GCAGCTCAGGTTCTG | 109 | GCCTACCGTGATGCTCTTCCCAT |
| | | GGAGA | | TCCCCAGAACC |
| R2 v1 | 82 | GATCAGTGAAACGCA | 110 | GCTAGCATCAGAGTTGGACACAT |
| | | CCAGA | | GGAACGTTGGGTGCGTTTCACT |
| R2 v2 | 82 | GATCAGTGAAACGCA | 111 | GCCTACCGTGATGCTCTTCCCAT |
| | | CCAGA | | TCGGTGCGTTTCACT |

T indicates the presence of uracil.

TABLE 6
Sequences of dsDNA donor with 3' overhangs used.

TRAC_SA-2A-EGFP_v1
TACCGTACACCAATGAGACCGCAATGACCAGCT*G*C*T*G*ACCTCTTCTCTTCCTCCCACAGATATCGG
ATCCGGCGCCACAAATTTCAGCCTGCTGAAACAGGCCGGCGACGTGGAAGAGAACCCCGGCCCTTCCA
TGGTGAGCAAGGGCGAGGAGCTGTTCACCGGGGTGGTGCCCATCCTGGTCGAGCTGGACGGCGACGT
AAACGGCCACAAGTTCAGCGTGTCCGGCGAGGGCGAGGGCGATGCCACCTACGGCAAGCTGACCCTG
AAGTTCATCTGCACCACCGGCAAGCTGCCCGTGCCCTGGCCCACCCTCGTGACCACCCTGACCTACGGC
GTGCAGTGCTTCAGCCGCTACCCCGACCACATGAAGCAGCACGACTTCTTCAAGTCCGCCATGCCCGAA
GGCTACGTCCAGGAGCGCACCATCTTCTTCAAGGACGACGGCAACTACAAGACCCGCGCCGAGGTGA
AGTTCGAGGGCGACACCCTGGTGAACCGCATCGAGCTGAAGGGCATCGACTTCAAGGAGGACGGCAA
CATCCTGGGGCACAAGCTGGAGTACAACTACAACAGCCACAACGTCTATATCATGGCCGACAAGCAGAA
GAACGGCATCAAGGTGAACTTCAAGATCCGCCACAACATCGAGGACGGCAGCGTGCAGCTCGCCGAC
CACTACCAGCAGAACACCCCCATCGGCGACGGCCCCGTGCTGCTGCCCGACAACCACTACCTGAGCAC
CCAGTCCGCCCTGAGCAAAGACCCCAACGAGAAGCGCGATCACATGGTCCTGCTGGAGTTCGTGACCG
CCGCCGGGATCACTCTCGGCATGGACGAGCTGTACAAGTGATAACCTGCAGGCTGTGCCTTCTAGTTGC
CAGCCATCTGTTGTTTGCCCCTCCCCCGTGCCTTCCTTGACCCTGGAAGGTGCCACTCCCACTGTCCTTT
CCTAATAAAATGAGGAAATTGCATCGCATTGTCTGAGTAGGTGTCATTCTATTCTGGGGGGTGGGGTGG
GGCAGGACAGCAAGGGGGAGGATTGGGAAGACAATAGCAGGCATGCTGGGGATGCGGTGGGCTC*T
*A*T*G*GGCTAGCATCAGAGTTGGACACATGGAACGTTG (SEQ ID NO: 112)
TRAC_SA-2A-EGFP_v2
TACCGTACACCAATGAGACCGGAACT*G*C*T*G*ACCTCTTCTCTTCCTCCCACAGATATCGGATCCGG
CGCCACAAATTTCAGCCTGCTGAAACAGGCCGGCGACGTGGAAGAGAACCCCGGCCCTTCCATGGTGA
GCAAGGGCGAGGAGCTGTTCACCGGGGTGGTGCCCATCCTGGTCGAGCTGGACGGCGACGTAAACG
GCCACAAGTTCAGCGTGTCCGGCGAGGGCGAGGGCGATGCCACCTACGGCAAGCTGACCCTGAAGTT
CATCTGCACCACCGGCAAGCTGCCCGTGCCCTGGCCCACCCTCGTGACCACCCTGACCTACGGCGTGCA
GTGCTTCAGCCGCTACCCCGACCACATGAAGCAGCACGACTTCTTCAAGTCCGCCATGCCCGAAGGCTA
CGTCCAGGAGCGCACCATCTTCTTCAAGGACGACGGCAACTACAAGACCCGCGCCGAGGTGAAGTTCG
AGGGCGACACCCTGGTGAACCGCATCGAGCTGAAGGGCATCGACTTCAAGGAGGACGGCAACATCCT
GGGGCACAAGCTGGAGTACAACTACAACAGCCACAACGTCTATATCATGGCCGACAAGCAGAAGAACG
GCATCAAGGTGAACTTCAAGATCCGCCACAACATCGAGGACGGCAGCGTGCAGCTCGCCGACCACTAC
CAGCAGAACACCCCCATCGGCGACGGCCCCGTGCTGCTGCCCGACAACCACTACCTGAGCACCCAGTC
CGCCCTGAGCAAAGACCCCAACGAGAAGCGCGATCACATGGTCCTGCTGGAGTTCGTGACCGCCGCCG
GGATCACTCTCGGCATGGACGAGCTGTACAAGTGATAACCTGCAGGCTGTGCCTTCTAGTTGCCAGCCA
TCTGTTGTTTGCCCCTCCCCCGTGCCTTCCTTGACCCTGGAAGGTGCCACTCCCACTGTCCTTTCCTAATA
AAATGAGGAAATTGCATCGCATTGTCTGAGTAGGTGTCATTCTATTCTGGGGGGGGGGTGGGGCAGG
ACAGCAAGGGGGAGGATTGGGAAGACAATAGCAGGCATGCTGGGGATGCGGTGGGCTC*T*A*T*G*
GGCCTACCGTGATGCTCTTCCCATTC (SEQ ID NO: 113)
IL2RG-EGFP
TACCGTACACCTTAGAGACCGCATAGACCAGCG*C*C*A*C*CATGGTGAGCAAGGGCGAGGAGCTGT
TCACCGGGGTGGTGCCCATCCTGGTCGAGCTGGACGGCGACGTAAACGGCCACAAGTTCAGCGTGTCC
GGCGAGGGCGAGGGCGATGCCACCTACGGCAAGCTGACCCTGAAGTTCATCTGCACCACCGGCAAGC
TGCCCGTGCCCTGGCCCACCCTCGTGACCACCCTGACCTACGGCGTGCAGTGCTTCAGCCGCTACCCCG
ACCACATGAAGCAGCACGACTTCTTCAAGTCCGCCATGCCCGAAGGCTACGTCCAGGAGCGCACCATCT
TCTTCAAGGACGACGGCAACTACAAGACCCGCGCCGAGGTGAAGTTCGAGGGCGACACCCTGGTGAA
CCGCATCGAGCTGAAGGGCATCGACTTCAAGGAGGACGGCAACATCCTGGGGCACAAGCTGGAGTAC
AACTACAACAGCCACAACGTCTATATCATGGCCGACAAGCAGAAGAACGGCATCAAGGTGAACTTCAA
GATCCGCCACAACATCGAGGACGGCAGCGTGCAGCTCGCCGACCACTACCAGCAGAACACCCCCATCG
GCGACGGCCCCGTGCTGCTGCCCGACAACCACTACCTGAGCACCCAGTCCGCCCTGAGCAAAGACCCC
AACGAGAAGCGCGATCACATGGTCCTGCTGGAGTTCGTGACCGCCGCCGGGATCACTCTCGGCATGGA
CGAGCTGTACAAGTGACCTGCAGGCTGTGCCTTCTAGTTGCCAGCCATCTGTTGTTTGCCCCTCCCCCGT
GCCTTCCTTGACCCTGGAAGGTGCCACTCCCACTGTCCTTTCCTAATAAAATGAGGAAATTGCATCGCAT
TGTCTGAGTAGGTGTCATTCTATTCTGGGGGGTGGGGTGGGGCAGGACAGCAAGGGGGAGGATTGGG
AAGACAATAGCAGGCATGCTGGGGATGCGGTGGGCTC*T*A*T*G*GGCTAGCATCAGAGTTGGACAC
ATGGAACGTTG (SEQ ID NO: 114)
AAVS1_SA-2A_EGFP_v1
TACCGTACACCAATGAGACCGCAATGACCAGCT*G*C*T*G*ACCTCTTCTCTTCCTCCCACAGGGATCC
GGCGCCACAAATTTCAGCCTGCTGAAACAGGCCGGCGACGTGGAAGAGAACCCCGGCCCTTCCATGG
TGAGCAAGGGCGAGGAGCTGTTCACCGGGGTGGTGCCCATCCTGGTCGAGCTGGACGGCGACGTAAA
CGGCCACAAGTTCAGCGTGTCCGGCGAGGGCGAGGGCGATGCCACCTACGGCAAGCTGACCCTGAAG
TTCATCTGCACCACCGGCAAGCTGCCCGTGCCCTGGCCCACCCTCGTGACCACCCTGACCTACGGCGTG
CAGTGCTTCAGCCGCTACCCCGACCACATGAAGCAGCACGACTTCTTCAAGTCCGCCATGCCCGAAGGC
TACGTCCAGGAGCGCACCATCTTCTTCAAGGACGACGGCAACTACAAGACCCGCGCCGAGGTGAAGTT
CGAGGGCGACACCCTGGTGAACCGCATCGAGCTGAAGGGCATCGACTTCAAGGAGGACGGCAACATC
CTGGGGCACAAGCTGGAGTACAACTACAACAGCCACAACGTCTATATCATGGCCGACAAGCAGAAGAA
CGGCATCAAGGTGAACTTCAAGATCCGCCACAACATCGAGGACGGCAGCGTGCAGCTCGCCGACCACT
ACCAGCAGAACACCCCCATCGGCGACGGCCCCGTGCTGCTGCCCGACAACCACTACCTGAGCACCCAG
TCCGCCCTGAGCAAAGACCCCAACGAGAAGCGCGATCACATGGTCCTGCTGGAGTTCGTGACCGCCGC
CGGGATCACTCTCGGCATGGACGAGCTGTACAAGTGACCTGCAGGCTGTGCCTTCTAGTTGCCAGCCAT
CTGTTGTTTGCCCCTCCCCCGTGCCTTCCTTGACCCTGGAAGGTGCCACTCCCACTGTCCTTTCCTAATAA
AATGAGGAAATTGCATCGCATTGTCTGAGTAGGTGTCATTCTATTCTGGGGGGTGGGGTGGGGCAGGA
CAGCAAGGGGGAGGATTGGGAAGACAATAGCAGGCATGCTGGGGATGCGGTGGGCTC*T*A*T*G*G
GCTAGCATCAGAGTTGGACACATGGAACGTTG (SEQ ID NO: 115)
AAVS1_SA-2A_EGFP_v2
TACCGTACACCAATGAGACCGGAACT*G*C*T*G*ACCTCTTCTCTTCCTCCCACAGGGATCCGGCGCCA
CAAATTTCAGCCTGCTGAAACAGGCCGGCGACGTGGAAGAGAACCCCGGCCCTTCCATGGTGAGCAA
GGGCGAGGAGCTGTTCACCGGGGTGGTGCCCATCCTGGTCGAGCTGGACGGCGACGTAAACGGCCAC
AAGTTCAGCGTGTCCGGCGAGGGCGAGGGCGATGCCACCTACGGCAAGCTGACCCTGAAGTTCATCT
GCACCACCGGCAAGCTGCCCGTGCCCTGGCCCACCCTCGTGACCACCCTGACCTACGGCGTGCAGTGC
TTCAGCCGCTACCCCGACCACATGAAGCAGCACGACTTCTTCAAGTCCGCCATGCCCGAAGGCTACGTC
CAGGAGCGCACCATCTTCTTCAAGGACGACGGCAACTACAAGACCCGCGCCGAGGTGAAGTTCGAGG
GCGACACCCTGGTGAACCGCATCGAGCTGAAGGGCATCGACTTCAAGGAGGACGGCAACATCCTGGG
GCACAAGCTGGAGTACAACTACAACAGCCACAACGTCTATATCATGGCCGACAAGCAGAAGAACGGCA
TCAAGGTGAACTTCAAGATCCGCCACAACATCGAGGACGGCAGCGTGCAGCTCGCCGACCACTACCAG
CAGAACACCCCCATCGGCGACGGCCCCGTGCTGCTGCCCGACAACCACTACCTGAGCACCCAGTCCGC
CCTGAGCAAAGACCCCAACGAGAAGCGCGATCACATGGTCCTGCTGGAGTTCGTGACCGCCGCCGGG
ATCACTCTCGGCATGGACGAGCTGTACAAGTGACCTGCAGGCTGTGCCTTCTAGTTGCCAGCCATCTGT
TGTTTGCCCCTCCCCCGTGCCTTCCTTGACCCTGGAAGGTGCCACTCCCACTGTCCTTTCCTAATAAAATG
AGGAAATTGCATCGCATTGTCTGAGTAGGTGTCATTCTATTCTGGGGGGTGGGGTGGGGCAGGACAGC
AAGGGGGAGGATTGGGAAGACAATAGCAGGCATGCTGGGGATGCGGTGGGCTC*T*A*T*G*GGCCT
ACCGTGATGCTCTTCCCATTC (SEQ ID NO: 116)
Phosphorothioate linkages, which blocks lambda exonuclease, are highlighted with a star (*).

TABLE 7
Sequences of ssDNA donors used.

AAVS1_SA-2A-EGFP_Top_v1
CTCGTGACCACCCTGACCTACGGCGTGCAGTGCTTCAGCCGCTACCCCGACCACATGAAGCAGCACGAC
TTCTTCAAGTCCGCCATGCCCGAAGGCTACGTCCAGGAGCGCACCATCTTCTTCAAGGACGACGGCAAC
TACAAGACCCGCGCCGAGGTGAAGTTCGAGGGCGACACCCTGGTGAACCGCATCGAGCTGAAGGGCA
TCGACTTCAAGGAGGACGGCAACATCCTGGGGCACAAGCTGGAGTACAACTACAACAGCCACAACGTC
TATATCATGGCCGACAAGCAGAAGAACGGCATCAAGGTGAACTTCAAGATCCGCCACAACATCGAGGA
CGGCAGCGTGCAGCTCGCCGACCACTACCAGCAGAACACCCCCATCGGCGACGGCCCCGTGCTGCTGC
CCGACAACCACTACCTGAGCACCCAGTCCGCCCTGAGCAAAGACCCCAACGAGAAGCGCGATCACATG
GTCCTGCTGGAGTTCGTGACCGCCGCCGGGATCACTCTCGGCATGGACGAGCTGTACAAGTGACCTGC
AGGCTGTGCCTTCTAGTTGCCAGCCATCTGTTGTTTGCCCCTCCCCCGTGCCTTCCTTGACCCTGGAAGG
TGCCACTCCCACTGTCCTTTCCTAATAAAATGAGGAAATTGCATCGCATTGTCTGAGTAGGTGTCATTCTA
TTCTGGGGGGTGGGGTGGGGCAGGACAGCAAGGGGGAGGATTGGGAAGACAATAGCAGGCATGCT
GGGGATGCGGTGGGCTCTATGGGCTAGCATCAGAGTTGGACACATGGAACGTTG (SEQ ID NO: 117)
AAVS1_SA-2A-EGFP_Top_v2
GGGCATCGACTTCAAGGAGGACGGCAACATCCTGGGGCACAAGCTGGAGTACAACTACAACAGCCAC
AACGTCTATATCATGGCCGACAAGCAGAAGAACGGCATCAAGGTGAACTTCAAGATCCGCCACAACATC
GAGGACGGCAGCGTGCAGCTCGCCGACCACTACCAGCAGAACACCCCCATCGGCGACGGCCCCGTGC
TGCTGCCCGACAACCACTACCTGAGCACCCAGTCCGCCCTGAGCAAAGACCCCAACGAGAAGCGCGAT
CACATGGTCCTGCTGGAGTTCGTGACCGCCGCCGGGATCACTCTCGGCATGGACGAGCTGTACAAGTG
ACCTGCAGGCTGTGCCTTCTAGTTGCCAGCCATCTGTTGTTTGCCCCTCCCCCGTGCCTTCCTTGACCCT
GGAAGGTGCCACTCCCACTGTCCTTTCCTAATAAAATGAGGAAATTGCATCGCATTGTCTGAGTAGGTGT
CATTCTATTCTGGGGGGTGGGGTGGGGCAGGACAGCAAGGGGGAGGATTGGGAAGACAATAGCAGG
CATGCTGGGGATGCGGTGGGCTCTATGGGCTAGCATCAGAGTTGGACACATGGAACGTTG (SEQ ID
NO: 118)
AAVS1_SA-2A-EGFP_Bot_v1
GCGGTCACGAACTCCAGCAGGACCATGTGATCGCGCTTCTCGTTGGGGTCTTTGCTCAGGGCGGACTG
GGTGCTCAGGTAGTGGTTGTCGGGCAGCAGCACGGGGCCGTCGCCGATGGGGGTGTTCTGCTGGTAG
TGGTCGGCGAGCTGCACGCTGCCGTCCTCGATGTTGTGGCGGATCTTGAAGTTCACCTTGATGCCGTTC
TTCTGCTTGTCGGCCATGATATAGACGTTGTGGCTGTTGTAGTTGTACTCCAGCTTGTGCCCCAGGATGT
TGCCGTCCTCCTTGAAGTCGATGCCCTTCAGCTCGATGCGGTTCACCAGGGTGTCGCCCTCGAACTTCA
CCTCGGCGCGGGTCTTGTAGTTGCCGTCGTCCTTGAAGAAGATGGTGCGCTCCTGGACGTAGCCTTCG
GGCATGGCGGACTTGAAGAAGTCGTGCTGCTTCATGTGGTCGGGGTAGCGGCTGAAGCACTGCACGC
CGTAGGTCAGGGTGGTCACGAGGGTGGGCCAGGGCACGGGCAGCTTGCCGGTGGTGCAGATGAACT
TCAGGGTCAGCTTGCCGTAGGTGGCATCGCCCTCGCCCTCGCCGGACACGCTGAACTTGTGGCCGTTTA
CGTCGCCGTCCAGCTCGACCAGGATGGGCACCACCCCGGTGAACAGCTCCTCGCCCTTGCTCACCATG
GAAGGGCCGGGGTTCTCTTCCACGTCGCCGGCCTGTTTCAGCAGGCTGAAATTTGTGGCGCCGGATCC
CTGTGGGAGGAAGAGAAGAGGTCAGCAGCTGGTCATTGCGGTCTCATTGGTGTACGGTA (SEQ ID
NO: 119)
AAVS1_SA-2A-EGFP_Bot_v2
GTTCTTCTGCTTGTCGGCCATGATATAGACGTTGTGGCTGTTGTAGTTGTACTCCAGCTTGTGCCCCAGG
ATGTTGCCGTCCTCCTTGAAGTCGATGCCCTTCAGCTCGATGCGGTTCACCAGGGTGTCGCCCTCGAAC
TTCACCTCGGCGCGGGTCTTGTAGTTGCCGTCGTCCTTGAAGAAGATGGTGCGCTCCTGGACGTAGCCT
TCGGGCATGGCGGACTTGAAGAAGTCGTGCTGCTTCATGTGGTCGGGGTAGCGGCTGAAGCACTGCA
CGCCGTAGGTCAGGGTGGTCACGAGGGTGGGCCAGGGCACGGGCAGCTTGCCGGTGGTGCAGATGA
ACTTCAGGGTCAGCTTGCCGTAGGTGGCATCGCCCTCGCCCTCGCCGGACACGCTGAACTTGTGGCCG
TTTACGTCGCCGTCCAGCTCGACCAGGATGGGCACCACCCCGGTGAACAGCTCCTCGCCCTTGCTCACC
ATGGAAGGGCCGGGGTTCTCTTCCACGTCGCCGGCCTGTTTCAGCAGGCTGAAATTTGTGGCGCCGGA
TCCCTGTGGGAGGAAGAGAAGAGGTCAGCAGCTGGTCATTGCGGTCTCATTGGTGTACGGTA (SEQ ID
NO: 120)

### Other Embodiments

From the foregoing description, it will be apparent that variations and modifications may be made to the invention described herein to adapt it to various usages and conditions. Such embodiments are also within the scope of the following claims.

The recitation of a listing of elements in any definition of a variable herein includes definitions of that variable as any single element or combination (or subcombination) of listed elements. The recitation of an embodiment herein includes that embodiment as any single embodiment or in combination with any other embodiments or portions thereof.

All patents and publications mentioned in this specification are herein incorporated by reference to the same extent as if each independent patent and publication was specifically and individually indicated to be incorporated by reference.

What is claimed is:

1. A genome editing system comprising:
i) a nucleic acid programmable DNA binding protein having DNA nickase activity;
ii) a reverse transcriptase;
iii) a pair of prime editing guide RNA (pegRNA), wherein each member of the pair comprises
a spacer sequence complementary to a target polynucleotide sequence,
a reverse transcriptase template, wherein the reverse transcriptase template has less than about 85% identity to the target polynucleotide sequence, and
a primer binding sequence comprising a region complementary to a region upstream of a nick site in the double-stranded target DNA sequence; and
iv) a donor sequence comprising a 3′ overhang that hybridizes to a region of the reverse transcriptase template.

2. The genome editing system of claim 1, wherein the nucleic acid programmable DNA binding protein comprises a CAS domain selected from the group consisting of a CAS9, CAS12a, and a CPF1 domain.

3. The genome editing system of claim 1, wherein the nucleic acid programmable DNA binding protein is fused to the reverse transcriptase.

4. The genome editing system of claim 1, wherein the 3′ end of the first pegRNA binds the target gene at a site that is at least about 35 base pairs from the 3′ end of the site where the second pegRNA binds.

5. The genome editing system of claim 1, wherein the reverse transcriptase template have less than about 80% nucleic acid sequence identity with the target polynucleotide sequence.

6. The genome editing system of claim 1, wherein the reverse transcriptase template is between 10 to 40 nucleotides in length.

7. The genome editing system of claim 1, further comprising a viral particle X (VPX) and/or exogenous dNTPs.

8. The genome editing system of claim 6, wherein the stabilizing sequence is a linker sequence and/or pseudo-knot sequence.

9. The genome editing system of claim 1, wherein the 3′ overhang is between 15 to 10,000 nucleotides in length.

10. A method for editing a target polynucleotide, the method comprising contacting the genome editing systems of claim 1 with a target polynucleotide, thereby editing the target polynucleotide.

11. A method for editing a target genome, the method comprising contacting the target genome with the genome editing system of claim 1, thereby editing the target genome.

12. A method of editing a TINF2 polynucleotide in a cell, the method comprising contacting the cell with a genome editing system comprising:
i) a nucleic acid programmable DNA binding protein having DNA nickase activity;
ii) a reverse transcriptase;
iii) a pair of prime editing guide RNA (pegRNA), each comprising a spacer sequence, a reverse transcriptase template, wherein the reverse transcriptase template has less than about 85% identity to the double-stranded target polynucleotide sequence, and a primer binding sequence comprising a region complementary to a region upstream of a nick site in the double-stranded target DNA sequence, wherein the spacer sequence of the first member of the pair comprises a sequence that is complementary to a sequence 5′ of the sequence that encodes the TINF2 DC cluster and the second member of the pair comprises a spacer sequence that is complementary to a sequence 3′ of the sequence that encodes the TINF2 DC cluster; and
iv) a donor sequence comprising a 3′ overhang that is complementary to at least a portion of the target sequence, thereby editing the TINF2 polynucleotide.

13. A method of editing an ATP1A1 polynucleotide in a cell, the method comprising contacting the cell with a genome editing system comprising:
i) a nucleic acid programmable DNA binding protein having DNA nickase activity;
ii) a reverse transcriptase;
iii) a pair of prime editing guide RNA (pegRNA), each comprising a spacer sequence, a reverse transcriptase template, wherein the reverse transcriptase template has less than about 85% identity to the double-stranded target polynucleotide sequence, and a primer binding sequence comprising a region complementary to a region upstream of a nick site in the double-stranded target DNA sequence, wherein the spacer sequence of the first member of the pair comprises a sequence that is complementary to a sequence 5′ of a target nucleotide sequence in an ATP1A1 locus and the second member of the pair comprises a spacer sequence that is complementary to a sequence 3′ of a target sequence in an ATP1A1 locus; and
iv) a donor sequence comprising a 3′ overhang that hybridizes to a region of the reverse transcriptase template, thereby editing the ATP1A1 polynucleotide.

14. A method of inserting a donor sequence at a TRAC locus in a cell, the method comprising contacting the cell with a genome editing system comprising:
i) a nucleic acid programmable DNA binding protein having DNA nickase activity;
ii) a reverse transcriptase;
iii) a pair of prime editing guide RNA (pegRNA), each comprising a spacer sequence, a reverse transcriptase template, wherein the reverse transcriptase template has less than about 85% identity to the double-stranded target polynucleotide sequence, and a primer binding sequence comprising a region complementary to a region upstream of a nick site in the double-stranded target DNA sequence, wherein the spacer sequence of the first member of the pair comprises a sequence that is complementary to a sequence 5′ of a target nucleotide sequence in a TRAC locus and the second member of the pair comprises a spacer sequence that is complementary to a sequence 3′ of a target sequence in a TRAC locus; and
iv) a donor sequence comprising a 3′ overhang that hybridizes to a region of the reverse transcriptase template, thereby inserting the donor sequence at the TRAC locus in the cell.

15. A method of editing an IL2RG polynucleotide in a cell, the method comprising contacting the cell with a genome editing system comprising:
i) a nucleic acid programmable DNA binding protein having DNA nickase activity;
ii) a reverse transcriptase;
iii) a pair of prime editing guide RNA (pegRNA), each comprising a spacer sequence, a reverse transcriptase template, wherein the reverse transcriptase template has less than about 85% identity to the double-stranded target polynucleotide sequence, and a primer binding sequence comprising a region complementary to a region upstream of a nick site in the double-stranded target DNA sequence, wherein the spacer sequence of the first member of the pair comprises a sequence that is complementary to a sequence 5′ of a target nucleotide sequence in an IL2RG locus and the second member of the pair comprises a spacer sequence that is complementary to a sequence 3′ of a target sequence in an IL2RG locus; and
iv) a donor sequence comprising a 3′ overhang that hybridizes to a region of the reverse transcriptase template, thereby editing the IL2RG polynucleotide.

16. A method of inserting a donor sequence at an AAVS1 locus in a cell, the method comprising contacting the cell with a genome editing system comprising:
i) a nucleic acid programmable DNA binding protein having DNA nickase activity;
ii) a reverse transcriptase;
iii) a pair of prime editing guide RNA (pegRNA), each comprising a spacer sequence, a reverse transcriptase template, wherein the reverse transcriptase template has less than about 85% identity to the double-stranded target polynucleotide sequence, and a primer binding sequence comprising a region complementary to a region upstream of a nick site in the double-stranded target DNA sequence, wherein the spacer sequence of the first member of the pair comprises a sequence that is complementary to a sequence 5′ of a target nucleotide sequence in an AAVS1 locus and the second member of the pair comprises a spacer sequence that is complementary to a sequence 3′ of a target sequence in an AAVS1 locus; and
iv) a donor sequence comprising a 3′ overhang that hybridizes to a region of the reverse transcriptase template, thereby inserting the donor sequence at the AAVS1 locus in the cell.

17. The method of claim 16, wherein the cell is derived from a subject having dyskeratosis congenita (DC).

18. A method of treating DC in a subject, the method comprising administering a paired prime editing system to the subject, wherein the paired prime editing system comprises a prime editor comprising a napDNAbp, a reverse transcriptase, and a pair of pegRNAs, each comprising a spacer sequence complementary to a TINF2 polynucleotide comprising a mutation associated with DC, a primer binding sequence (PBS), and a reverse transcriptase template that encodes a wild-type TINF2 polynucleotide, or a fragment thereof.

19. The method of claim 18, wherein the wild-type TINF2 polynucleotide, or a fragment thereof, encodes a TINF2 DC cluster.

20. A polynucleotide comprising a sequence present in any one of Tables 1-4, 6, or 7.
